(19) United States
(12) Patent Application Publication
(10) Pub. No.: US 2016/0002269 A1
(43) Pub. Date: Jan. 7, 2016

(54) LANTHANIDE CLUSTERS AND METHODS OF USE THEREOF

(71) Applicant: YEDA RESEARCH AND DEVELOPMENT CO. LTD., Rehovot (IL)

(72) Inventors: Abraham SHANZER, Rehovot (IL); Galina MELMAN, Rehovot (IL); Yaniv BARDA, Rehovot (IL); Raghavendra KIKKERI, Rehovot (IL); Boris KRAIZ, Rehovot (IL); Larissa GINAT, Rehovot (IL)

(21) Appl. No.: 14/766,996

(22) PCT Filed: Feb. 13, 2014
(86) PCT No.: PCT/IL2014/050147

§ 371 Date: Aug. 11, 2015

Related U.S. Application Data
(60) Provisional application No. 61/763,176, filed on Feb. 11, 2013.

(51) Int. Cl. C07F 5/00; C09D 11/38; G01N 21/64; C09K 11/06; A61K 49/10

(52) U.S. Cl. CPC C07F 5/003; C09K 11/06; A61K 49/106; G01N 21/6428; G01N 21/6486; C09D 11/38; C09K 2211/182; G01N 2021/6432

(57) ABSTRACT

The present invention is directed to multinuclear lanthanides chiral clusters, based on phenyl-oxazoline-amide (POxA) ligands, and to methods of use thereof. The chiral clusters of this invention are highly fluorescent with high stability.

### FIELD OF THE INVENTION

The present invention is directed to multinuclear lanthanides chiral clusters, based on phenyl-oxazoline-amide (POxA) ligands, and to methods of use thereof. The chiral clusters of this invention are highly fluorescent with high stability.

### BACKGROUND OF THE INVENTION

Lanthanide complexes posses unique optical and magnetic properties with applications as optical fibers, electroluminescent materials, luminescent bio-probes, ‘markers’ in encoding inks, new NMR shift reagents, contrast agents in magnetic resonance imaging (MRI), organ specific carriers for radioactive lanthanide isotopes and as single molecule magnets (SMM).

Lanthanides are attractive alternatives to organic chromophores which are currently used as markers in diverse applications from biological research tools to technologically relevant optronic devices. However, organic chromophores typically exhibit poor light durability (they undergo bleaching via a number of mechanisms), relatively broad singlet emission bands, and very short excitation decay times (nanoseconds) prompting the search for more effective/efficient ‘signaling’ tools. Lanthanide luminescence overcomes many of the shortcoming of organic dyes with its diversity of ions (fifteen lanthanide elements with similar, monotonically varying chemical properties, but different ionic radii as well as luminescent and magnetic properties), large Stokes shift, narrow emission spectral lines ranging from UV/Vis to the near infra-red (NIR), delayed emission (maximizing signal/noise by eliminating background signals from some amino acids and nucleotides), minor concentration quenching and long excitation decay times (in milliseconds) which render time-resolved spectroscopy an extremely powerful research tool.

Lanthanide luminescence is characterized by low Quantum Yield (QY) arising from several mechanisms including (i) forbidden f-f transition and (ii) deactivations of excited states (particularly in the NIR range) by non-radiative processes. Methodologies to overcome the limitation of direct excitation have been developed, by incorporating chromophores in the vicinity of the lanthanide ions, known to form the ‘antenna effect’ for indirect excitation.

Luminescence quenching reduces the excited state lifetimes and consequently the quantum yields. The most common lanthanide luminescence quenching occur by non-radiative relaxation of the excited state (excited state deactivation), which originate from the O—H vibrational overtones of water molecules bound to the inner and outer spheres of the chelator and other N—H and C—H group oscillations. Means to overcoming these limitations are based on exchanging OH, NH, and CH groups by deuterium (OD, ND and CD) or fluorinated analogs are well documented. However, these solutions are associated with extensive synthetic labor and are not readily applicable for practical applications.

The magnetic properties of lanthanide ions are widely applied in diverse fields such as magnet technology, magnetic liquid crystals, magnetic refrigeration and contrast agents in Magnetic Resonance Imaging (MRI). The last application utilizes paramagnetic lanthanide complexes as contrast agents by altering the relaxation times of water protons to improve soft tissue discrimination. The most widely used contrast enhancements in clinical practice (more then 95%) are thermodynamically and kinetically stable low molecular weight mono-Gadolinium (III) based complexes. The development of new imaging methodologies such as Chemical Exchange Saturation Transfer (CEST) and Paramagnetic Chemical Exchange Saturation Transfer (PARACEST), allows utilizing additional lanthanide ions (e.g. Europium and Dysprosium) and demands the design and synthesis of lanthanide chelators with improved relaxivity properties.

Lanthanide complexes can be also applied both for diagnostic and therapeutic purposes in nuclear medicine. Radiopharmaceutical uses lanthanide radionuclides with short half life-time, high yield of β-rays, which do not have high γ-emission (not to cause excessive tissue irradiation). Several lanthanides possess properties that fulfill these requirements and can be used for imaging (¹⁴¹Ce, ¹⁵³Gd, ¹⁶¹Tb, etc.) and therapeutic purposes (¹⁵³Sm, ⁹⁰Y and the ¹⁶⁶Dy/¹⁶⁶Ho pair).

This invention is directed to lanthanides chiral clusters and methods of use thereof, with high stability and high luminescence.

### SUMMARY OF THE INVENTION

In one embodiment, this invention is directed to a multinuclear lanthanide chiral cluster comprising phenyl-oxazoline-amide (POxA) ligand or salt thereof represented by the structure of formula IA:

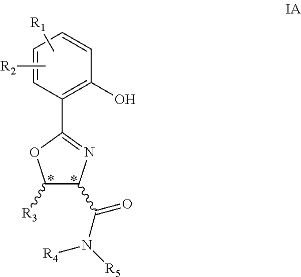

and lanthanide(III) ions;
wherein,

R₁ is hydrogen, alkyl, alkenyl, alkyldiazo, aryldiazo, alkynyl, aryl, halogen, —C≡C-Ph-R, —N═N-Ph-R, CN, NH₂, OH, N₃, NO₂, COOH, COOR, SO₃H, SO₃R, SO₂NHR, O-alkyl, alkylamino, haloalkyl, or R₁ and R₂ combine together to form a 5-7 membered ring; wherein said 5-7 membered ring is saturated or unsaturated cycloalkyl or heterocycle; wherein said alkyl, alkenyl, alkyldiazo, aryldiazo, alkynyl, aryl and saturated or unsaturated cycloalkyl or heterocycle is substituted or unsubstituted;

R₂ is hydrogen, alkyl, alkenyl, alkyldiazo, aryldiazo, alkynyl, aryl, halogen, CN, NH₂, OH, N₃, NO₂, COOH, COOR, SO₃H, SO₃R, SO₂NHR, O-alkyl, alkylamino, haloalkyl, or R₁ and R₂ combine together to form a 5-7 membered ring; wherein said 5-7 membered ring is saturated or unsatureated cycloalkyl or heterocycle; wherein said alkyl, alkenyl, alkyldiazo, aryldiazo, alkynyl aryl, and saturated or unsaturated cycloalkyl or heterocycle is substituted or unsubstituted;

R₃ is alkyl, aryl, alkenyl, alkyldiazo, aryldiazo, alkynyl, halogen, CN, NH₂, OH, N₃, NO₂, COOH, SO₃H, SO₃R, SO₂NHR, COOR, O-alkyl, alkylamino or haloalkyl; wherein said alkyl, alkenyl, alkyldiazo, aryldiazo, alkynyl and aryl is substituted or unsubstituted;

R₄ is alkyl, alkenyl, alkyldiazo, aryldiazo, alkynyl, polyethylene glycol (PEG), sugars, glucose, manose, proteins, antibody, peptide, —CHR′COR, saturated or unsaturated cycloalkyl or heterocycle, or R₄ and R₅ combine together with the nitrogen to form a 5-7 membered ring; wherein said 5-7 membered ring is saturated or unsaturated cycloalkyl or heterocycle; wherein said alkyl, alkenyl, alkyldiazo, aryldiazo, alkynyl and saturated or unsaturated cycloalkyl or heterocycle is substituted or unsubstituted;

R₅ is hydrogen, alkyl, alkenyl or alkynyl or R₅ and R₄ combine together with the nitrogen to form a 5-7 membered ring; wherein said 5-7 membered ring is saturated or unsaturated cycloalkyl or heterocycle;

R is hydrogen, alkyl, alkylamine, —N(Alkyl)₂, alkenyl, alkynyl or saturated or unsaturated cycloalkyl or heterocycle; wherein said alkyl, alkenyl, alkynyl and saturated or unsaturated cycloalkyl or heterocycle is substituted or unsubstituted; and

R′ is an amino acid residue.

In one embodiment, this invention is directed to a multinuclear lanthanide chiral cluster comprising phenyl-oxazoline-amide(POxA) ligand or salt thereof represented by the structure of formula IIIA:

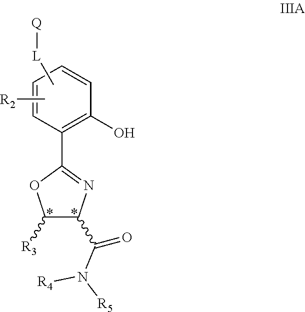

and lanthanide (III) ions;
wherein,
Q is a sensor, monomeric building-block for polymerization, a polymer, chromophore, surface adhesive group or combination thereof;
L is a bond or a linker;

R₂ is hydrogen, alkyl, alkenyl, alkyldiazo, aryldiazo, alkynyl, aryl, halogen, —C≡C-Ph-R, —N═N-Ph-R, CN, NH₂, OH, N₃, NO₂, COOH, COOR, SO₃H, SO₃R, SO₂NHR, O-alkyl, alkylamino, haloalkyl, or R₁ and R₂ combine together to form a 5-7 membered ring; wherein said 5-7 membered ring is saturated or unsatureated cycloalkyl or heterocycle; wherein said alkyl, alkenyl, alkyldiazo, aryldiazo, alkynyl, aryl and saturated or unsaturated cycloalkyl or heterocycle is substituted or unsubstituted;

R₃ is alkyl, aryl, alkenyl, alkyldiazo, aryldiazo, alkynyl, halogen, CN, NH₂, OH, N₃, NO₂, COOH, SO₃H, SO₃R, SO₂NHR, COOR, O-alkyl, alkylamino or haloalkyl; wherein said alkyl, alkenyl, alkyldiazo, aryldiazo, alkynyl and aryl is substituted or unsubstituted;

R₄ is alkyl, alkenyl, alkyldiazo, aryldiazo, alkynyl, polyethylene glycol (PEG), sugars, glucose, manose, galactose, proteins, antibody, peptide, —CHR′COR, saturated or unsaturated cycloalkyl or heterocycle; or R₄ and R₅ combine together with the nitrogen to form a 5-7 membered ring; wherein said 5-7 membered ring is saturated or unsaturated cycloalkyl or heterocycle; wherein said alkyl, alkenyl, alkyldiazo, aryldiazo, alkynyl and saturated or unsaturated cycloalkyl or heterocycle is substituted or unsubstituted;

R₅ is hydrogen, alkyl, alkenyl or alkynyl or R₅ and R₄ combine together with the nitrogen to form a 5-7 membered ring; wherein said 5-7 membered ring is saturated or unsaturated cycloalkyl;

R is hydrogen, alkyl, alkylamine, —N(Alkyl)₂, alkenyl, alkynyl or saturated or unsaturated cycloalkyl or heterocycle; wherein said alkyl, alkenyl, alkynyl and saturated or cycloalkyl or heterocycle is substituted or unsubstituted; and

R′ is an amino acid residue.

In one embodiment, the cluster of this invention further comprises oxygen based ligands, or halogens.

In one embodiment, this invention provides a three lanthanide chiral cluster as presented in FIGS. 2B, 2C, 3A, 3B, 4A, 4B, 5A or 5B.

In one embodiment, the chiral cluster of this invention is a three lanthanide cluster which coordinates to POxA ligand of this invention as presented by the structure of formula IIa and IIb; or as presented by the structure of formula IVa and IVb in equal ratios.

In one embodiment, this invention provides a seven lanthanide chiral cluster as presented in FIG. 2B.

In one embodiment, the chiral cluster of this invention is a seven lanthanide cluster which coordinates to POxA ligand of this invention as presented by the structure of formula IIa, IIb and IIc; or as presented by the structure of formula IVa, IVb and We in equal ratios.

In one embodiment, this invention provides an inkjet printing; or an optical fiber comprising the chiral cluster of this invention.

In one embodiment, this invention provides a biomarker comprising the chiral cluster of formula III of this invention.

In one embodiment, this invention provides a method of coding and reading coded information comprising writing a code with the chiral cluster of this invention, and reading said code by measuring its magnetic properties, its luminescence in visible or NIR or by measuring its emission light for circular polarized luminescence (CPL).

In one embodiment, this invention provides a method of identifying and quantifying a biomolecule in a sample, comprising:
(i) contacting a sample comprising a biomolecule with a chiral cluster of this invention wherein said biomolecule is selected from peptides, proteins, oligonucleotides, nucleic acids, oligosaccharides, polysaccharides, glycoproteins, phospholipids and enzymes; and
(ii) measuring luminescence following interaction between said biomolecule and said chiral cluster;
thereby identifying and quantifying said biomolecule in said sample.

In one embodiment, this invention provides a method of identifying and quantifying a metal ion in a sample, comprising:
(i) contacting a sample comprising a metal ion with a chiral cluster of this invention; and
(ii) measuring luminescence following interaction between said metal ion and said chiral cluster;
thereby identifying and quantifying said metal ion in said sample.

In one embodiment, this invention provides a contrast agent for Magnetic Resonance Imaging (MRI) comprising said multinuclear lanthanide chiral cluster of this invention.

In one embodiment, this invention provides a liquid crystal display comprising said multinuclear lanthanide chiral cluster of this invention.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a synthetic scheme of chiral ligands of this invention.

FIG. 2 depicts X-ray structure of the clusters of this invention. FIG. 2A depicts X-ray structure of 7Tb clusters derived from the trans POxA ligands. FIG. 2B depicts X-ray structure of 3Tb clusters derived from the cis POxA ligands. Identical structures were obtained for Tb, Sm, Pr, Dy, Gd, Ce and La lanthanides. When using a trans isomer a 7-Ln cluster is obtained, and when using a cis isomer a 3Ln cluster is obtained. FIG. 2C depicts X-ray structure of 3Tb clusters derived from the L-cis-(4S,5S) POxA ligands.

FIG. 3 depicts X-ray structure of iodo-3La clusters using iodo L-Cis-(4S,5S) POxA ligand. FIG. 3A depicts a top view of the 3La cluster. FIG. 3B depicts a side view of the iodo −3La cluster.

FIG. 4 depicts X-ray structure of acetylene −3Tb clusters (cis). FIG. 4A depicts a top view of the acetylene −3Tb cluster using 4-ethynyl L-cis (4S,5S) POxA ligand. FIG. 4B depicts a side view of the acetylene −3Tb cluster. FIG. 4C depicts a Circular Dichroism (CD) of 4-ethynyl L-cis (4S,5S) and 4-ethynyl D-cis (4R,5R) POxA ligands and their corresponding 3Tb clusters. FIG. 4D depicts ¹H NMR of 3La cluster derived from 4, ethynyl-L-cis (4S,5S) POxA ligand. ¹H NMR 500 MHz of the 3La cluster indicating the presence of two sets of peaks belonging to the same ligand in different chemical environments within the cluster. Two sets of the ligand are marked with corresponding numbers (10⁻⁵ M, CD₃OD). The upper structure present an expansion emphasising the relationship and relative intensities (1:1 ratio) between the different ligands within the 3La cluster.

FIG. 5 depicts X-ray structure of azido-3La clusters derived from 4-Azido D-cis (4R,5R) POxA ligand. FIG. 5A depicts a top view of the azido-3La clusters. FIG. 5B depicts a side view of the azido-3La clusters.

FIG. 6 depicts a 3Ln cluster using a cis isomer of the phenyl-oxazoline-amide ligand and its correspondent CD (top); and a 7Ln cluster using a trans isomer of the phenyl-oxazoline-amide ligand and its correspondent CD (bottom). The CD spectra of dissolved crystals (both enantiomers) in methanol.

FIG. 7 is a schematic presentation of 3D structures of 3Tb cluster having six cis POxA ligands of this invention and a 7Tb cluster having 9 trans PoxA ligands.

FIG. 8 depicts an amplified fluorescence in clusters, versus tripodal reference system.

FIG. 9 depicts Circularly Polarized Luminescence (CPL) emission (upper boxes) from several 3Ln clusters (3Tb, 3Dy, and 3Sm clusters) and total luminescence (lower boxes) spectra of L- and D-cis 3Dy POxA cluster, L- and D-cis 3Tb POxA cluster and L- and D-cis 3Sm POxA cluster (0.01 M) in MeOH at 295° K. gray: L-cis 3Ln POxA cluster, and black: D-cis 3Ln POxA cluster. A mirror-images relationship is observed in the CPL between enantiomers. Emissions from corresponding energy levels are marked below the boxes, as well as the excitation wavelength for each cluster. Two distinct emissions are observed from the 3Sm clusters.

FIG. 10 depicts fluorescence decay a tripodal-Tb complex vs. a 3Tb cluster of this invention. FIG. 10A presents the of a luminescence decay of D-cis Tb tripodal complex 0.075 mM upon excitation at 355 nm. Half life time of the compound can be derived from the equation t1/2=t1×ln2 and thus t1/2 of the cluster is 212755.2 pec. FIG. 10B presents luminescence decay of D-cis 3Tb POxA cluster 0.025 mM upon excitation at 355 nm. Half life time of the cluster can be derived from the equation t1/2=t1×ln2 and thus t1/2 of the cluster is 449185.22 μsec. The life-time of the cluster doubles that of the tripodal reference complex.

FIG. 11 depicts luminescence spectra of a tripodal-Tb complex vs. a 3Tb cluster of this invention. FIG. 11A presents fluorescence spectra of D-cis Tb tripodal complex 0.025 mM in MeOH (solid black) upon titration of 0.2 eq FeCl₃ (gray) and of 0.4 eq FeCl₃ (pale gray). FIG. 11B presents fluorescence spectra of D-cis-3Tb POxA cluster 0.025mM in MeOH (solid black) followed by titration with increased FeCl₃ concentration, from 0.2 eq-6 eq. A gradual decrease in luminescence was observed till 6 equivalents of FeCl₃, in mark difference from the reference tripodal structure (FIG. 12A).

FIG. 12 depicts magnetic properties of chiral clusters of this invention using using L-cis-(4S,5S) POxA ligand wherein 3Tb cluster provides 15.37 Bohr magneton and 7Tb cluster provides 22.37 Bohr magneton.

FIG. 13 depicts comparison between relaxivity of 3Gd clusters and Magnevist (GdDTPA) one of the most commonly used MRI contrast agent in medicine diagnostics.

FIG. 14 is a synthetic scheme of PEGylated phenyl-oxazoline-amide (POxA) ligand.

FIG. 15 depicts ¹H NMR 500 MHz of PEGylated cluster of this invention.

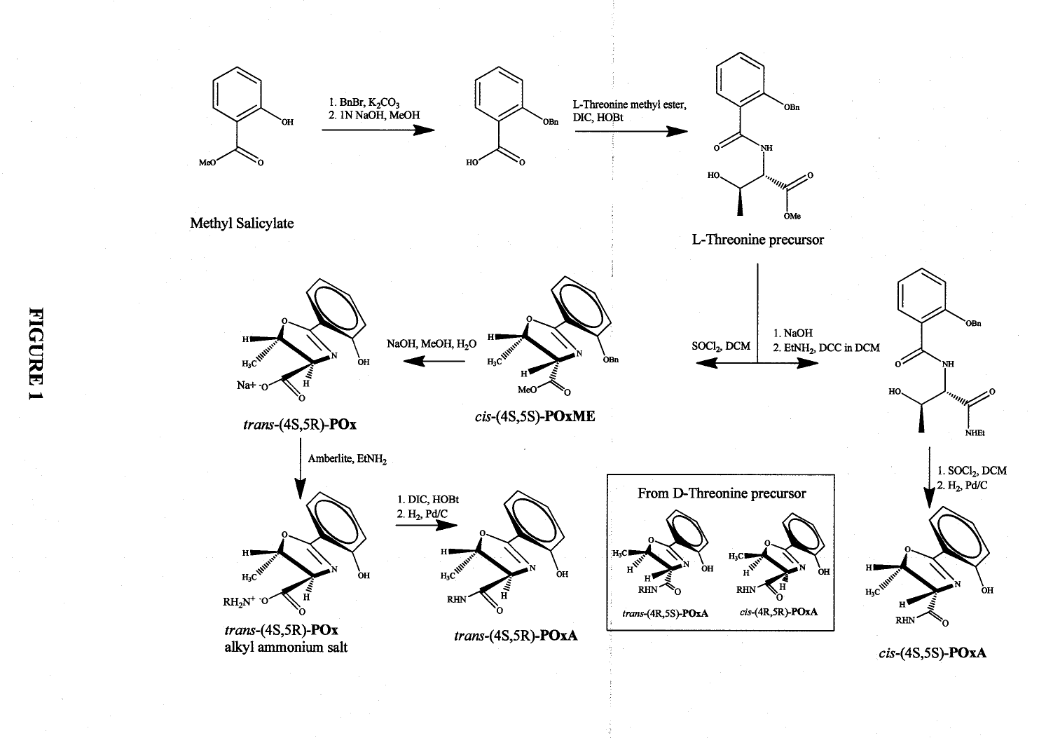

FIGS. A6A and 16B depict single crystal X-ray diffraction structure of 3La clusters derived from ((4S,5S)-2-(2-hydroxyphenyl)5-methyl-4,5-dihydrooxazole-4-yl)(morpholino)methanone (compound 119). A full structure (FIG. 16A) and a fragmented structure (FIG. 16B).

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In one embodiment, this invention is directed to a multinuclear lanthanides chiral cluster comprising phenyl-oxazoline-amide ligand or salt thereof represented by the structure of formula I:

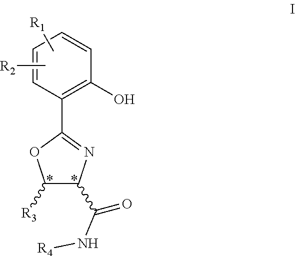

and lanthanide(III) ions;
wherein,

R₁ is hydrogen, alkyl, alkenyl, alkyldiazo, aryldiazo, alkynyl, aryl, halogen, —C≡C-Ph-R, —N═N-Ph-R, CN, NH₂, OH, N₃, NO₂, COOH, COOR, SO₃H, SO₃R, SO₂NHR, O-alkyl, alkylamino, haloalkyl, or R₁ and R₂ combine together to form a 5-7 membered ring; wherein said 5-7 membered ring is saturated or unsaturated cycloalkyl or heterocycle; wherein said alkyl, alkenyl, alkyldiazo, aryldiazo, alkynyl, aryl and saturated or unsaturated cycloalkyl or heterocycle is substituted or unsubstituted;

R₂ is hydrogen, alkyl, alkenyl, alkyldiazo, aryldiazo, alkynyl, aryl, halogen, CN, NH₂, OH, N₃, NO₂, COOH, COOR, SO₃H, SO₃R, SO₂NHR, O-alkyl, alkylamino, haloalkyl, or R₁ and R₂ combine together to form a 5-7 membered ring; wherein said 5-7 membered ring is saturated or unsatureated cycloalkyl or heterocycle; wherein said alkyl, alkenyl, alkyldiazo, aryldiazo, alkynyl aryl, and saturated or unsaturated cycloalkyl or heterocycle is substituted or unsubstituted;

R₃ is alkyl, aryl, alkenyl, alkyldiazo, aryldiazo, alkynyl, halogen, CN, NH₂, OH, N₃, NO₂, COOH, SO₃H, SO₃R, SO₂NHR, COOR, O-alkyl, alkylamino or haloalkyl; wherein said alkyl, alkenyl, alkyldiazo, aryldiazo, alkynyl and aryl is substituted or unsubstituted;

R₄ is alkyl, alkenyl, alkyldiazo, aryldiazo, alkynyl, polyethylene glycol (PEG), sugars, glucose, manose, proteins, antibody, peptide, -CHR′COR, saturated or unsaturated cycloalkyl or heterocycle; wherein said alkyl, alkenyl, alkyldiazo, aryldiazo, alkynyl and saturated or unsaturated cycloalkyl or heterocycle is substituted or unsubstituted;

R is hydrogen, alkyl, alkylamine, —N(Alkyl)₂, OH, alkenyl, alkynyl or saturated or unsaturated cycloalkyl or heterocycle; wherein said alkyl, alkenyl, alkynyl and saturated or unsaturated cycloalkyl or heterocycle is substituted or unsubstituted; and

R′ is an amino acid side chain.

In one embodiment, this invention is directed to a multinuclear lanthanide chiral cluster comprising phenyl-oxazoline-amide (POxA) ligand or salt thereof represented by the structure of formula IA:

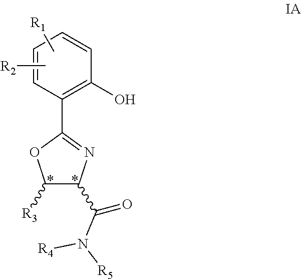

and lanthanide(III) ions;
wherein,

R₁ is hydrogen, alkyl, alkenyl, alkyldiazo, aryldiazo, alkynyl, aryl, halogen, —C≡C-Ph-R, —N═N-Ph-R, CN, NH₂, OH, N₃, NO₂, COOH, COOR, SO₃H, SO₃R, SO₂NHR, O-alkyl, alkylamino, haloalkyl, or R₁ and R₂ combine together to form a 5-7 membered ring; wherein said 5-7 membered ring is saturated or unsaturated cycloalkyl or heterocycle; wherein said alkyl, alkenyl, alkyldiazo, aryldiazo, alkynyl, aryl and saturated or unsaturated cycloalkyl or heterocycle is substituted or unsubstituted;

R₂ is hydrogen, alkyl, alkenyl, alkyldiazo, aryldiazo, alkynyl, aryl, halogen, CN, NH₂, OH, N₃, NO₂, COOH, COOR, SO₃H, SO₃R, SO₂NHR, O-alkyl, alkylamino, haloalkyl, or R₁ and R₂ combine together to form a 5-7 membered ring; wherein said 5-7 membered ring is saturated or unsatureated cycloalkyl or heterocycle; wherein said alkyl, alkenyl, alkyldiazo, aryldiazo, alkynyl aryl, and saturated or unsaturated cycloalkyl or heterocycle is substituted or unsubstituted;

R₃ is alkyl, aryl, alkenyl, alkyldiazo, aryldiazo, alkynyl, halogen, CN, NH₂, OH, N₃, NO₂, COOH, SO₃H, SO₃R, SO₂NHR, COOR, O-alkyl, alkylamino or haloalkyl; wherein said alkyl, alkenyl, alkyldiazo, aryldiazo, alkynyl and aryl is substituted or unsubstituted;

R₄ is alkyl, alkenyl, alkyldiazo, aryldiazo, alkynyl, polyethylene glycol (PEG), sugars, glucose, manose, proteins, antibody, peptide, -CHR′COR, saturated or unsaturated cycloalkyl or heterocycle, or R₄ and R₅ combine together with the nitrogen to form a 5-7 membered ring; wherein said 5-7 membered ring is saturated or unsaturated cycloalkyl or heterocycle; wherein said alkyl, alkenyl, alkyldiazo, aryldiazo, alkynyl and saturated or unsaturated cycloalkyl or heterocycle is substituted or unsubstituted;

R₅ is hydrogen, alkyl, alkenyl or alkynyl or R₅ and R₄ combine together with the nitrogen to form a 5-7 membered ring; wherein said 5-7 membered ring is saturated or unsaturated cycloalkyl or heterocycle;

R is hydrogen, alkyl, alkylamine, —N(Alkyl)₂, OH, alkenyl, alkynyl or saturated, or unsaturated cycloalkyl or heterocycle; wherein said alkyl, alkenyl, alkynyl and saturated or unsaturated cycloalkyl or heterocycle is substituted or unsubstituted; and

R′ is an amino acid side chain.

In one embodiment, this invention is directed to a multinuclear lanthanides chiral cluster comprising phenyl-oxazoline-amide (POxA) ligand or salt thereof wherein lanthanide ions coordinate to said POxA ligand as presented by the structure of formula IIa and the structure of formula IIb:

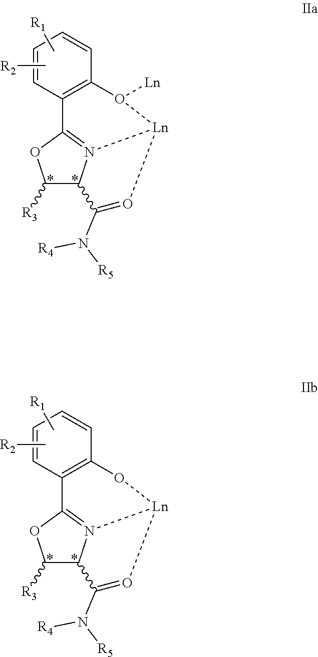

wherein,
Ln is a lanthanide(III) ion;
oxygen bridges coordinate between said lanthanide ions; and said cluster further comprises one or more oxygen based ligands, one or more halogens, or combination thereof.

R₁ is hydrogen, alkyl, alkenyl, alkyldiazo, aryldiazo, alkynyl, aryl, halogen, —C≡C-Ph-R, —N═N-Ph-R, CN, NH₂, OH, N₃, NO₂, COOH, COOR, SO₃H, SO₃R, SO₂NHR, O-alkyl, alkylamino, haloalkyl, or R₁ and R₂ combine together to form a 5-7 membered ring; wherein said 5-7 membered ring is saturated or unsaturated cycloalkyl or heterocycle; wherein said alkyl, alkenyl, alkyldiazo, aryldiazo, alkynyl, aryl and saturated or unsaturated cycloalkyl or heterocycle is substituted or unsubstituted;

R₂ is hydrogen, alkyl, alkenyl, alkyldiazo, aryldiazo, alkynyl, aryl, halogen, CN, NH₂, OH, N₃, NO₂, COOH, COOR, SO₃H, SO₃R, SO₂NHR, O-alkyl, alkylamino, haloalkyl, or R₁ and R₂ combine together to form a 5-7 membered ring; wherein said 5-7 membered ring is saturated or unsatureated cycloalkyl or heterocycle; wherein said alkyl, alkenyl, alkyldiazo, aryldiazo, alkynyl aryl, and saturated or unsaturated cycloalkyl or heterocycle is substituted or unsubstituted;

R₃ is alkyl, aryl, alkenyl, alkyldiazo, aryldiazo, alkynyl, halogen, CN, NH₂, OH, N₃, NO₂, COOH, SO₃H, SO₃R, SO₂NHR, COOR, O-alkyl, alkylamino or haloalkyl; wherein said alkyl, alkenyl, alkyldiazo, aryldiazo, alkynyl and aryl is substituted or unsubstituted;

R₄ is alkyl, alkenyl, alkyldiazo, aryldiazo, alkynyl, polyethylene glycol (PEG), sugars, glucose, manose, proteins, antibody, peptide, -CHR′COR, saturated or unsaturated cycloalkyl or heterocycle; or R₄ and R₅ combine together with the nitrogen to form a 5-7 membered ring; wherein said 5-7 membered ring is saturated or unsaturated cycloalkyl or heterocycle; wherein said alkyl, alkenyl, alkyldiazo, aryldiazo, alkynyl and saturated or unsaturated cycloalkyl or heterocycle is substituted or unsubstituted;

R₅ is hydrogen, alkyl, alkenyl or alkynyl or R₅ and R₄ combine together with the nitrogen to form a 5-7 membered ring; wherein said 5-7 membered ring is saturated or unsaturated cycloalkyl or heterocycle

R is hydrogen, alkyl, alkylamine, —N(Alkyl)₂, OH, alkenyl, alkynyl or saturated or unsaturated cycloalkyl or heterocycle; wherein said alkyl, alkenyl, alkynyl and saturated or unsaturated cycloalkyl or heterocycle is substituted or unsubstituted;

R′ is an amino acid side chain; and

wherein said cluster further comprises one or more oxygen based ligand, one or more halogens or combination thereof.

In another embodiment, the lanthanide ions of the 7Ln clusters further coordinate to said POxA ligand as presented by the structure of formula IIc:

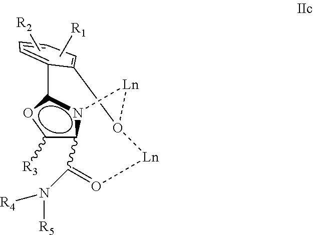

wherein R₁, R₂, R₃, R₄,R₅ are as described for the structure of formula IA; and Ln is a Ln(III) ion.

In another embodiment, multinuclear lanthanides chiral cluster of this invention includes three lanthanide ions. In another embodiment, multinuclear lanthanides chiral cluster of this invention includes seven lanthanide ions.

In one embodiment, the lanthanide ions of the three multinuclear lanthanide cluster coordinate to the POxA ligand of formula IA according to structures IIa and IIb in equal ratios.

In one embodiment, the lanthanide ions of the seven multinuclear lanthanide cluster coordinate to the POxA ligand of formula IA according to structures IIa, IIb and IIe in equal ratios.

In one embodiment, this invention is directed to a chiral multinuclear lanthanide cluster comprising phenyl-oxazoline-amide ligand or salt thereof represented by the structure of formula III:

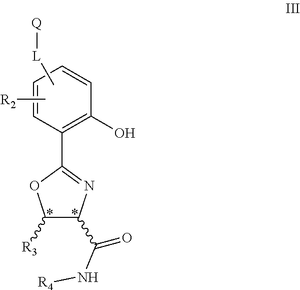

and lanthanide (III) ions;
wherein,

Q is a sensor, monomeric building-block for polymerization, a polymer, chromophore, surface adhesive group or combination thereof;

L is a bond or a linker;

R₂ is hydrogen, alkyl, alkenyl, alkyldiazo, aryldiazo, alkynyl, aryl, halogen, —C≡C-Ph-R, —N═N-Ph-R, CN, NH₂, OH, N₃, NO₂, COOH, COOR, SO₃H, SO₃R, SO₂NHR, O-alkyl, alkylamino, haloalkyl, or R₁ and R₂ combine together to form a 5-7 membered ring; wherein said 5-7 membered ring is saturated or unsatureated cycloalkyl or heterocycle; wherein said alkyl, alkenyl, alkyldiazo, aryldiazo, alkynyl, aryl and saturated or unsaturated cycloalkyl or heterocycle is substituted or unsubstituted;

R₃ is alkyl, aryl, alkenyl, alkyldiazo, aryldiazo, alkynyl, halogen, CN, NH₂, OH, N₃, NO₂, COOH, SO₃H, SO₃R, SO₂NHR, COOR, O-alkyl, alkylamino or haloalkyl; wherein said alkyl, alkenyl, alkyldiazo, aryldiazo, alkynyl and aryl is substituted or unsubstituted;

R₄ is alkyl, alkenyl, alkyldiazo, aryldiazo, alkynyl, polyethylene glycol (PEG), sugars, glucose, manose, galactose, proteins, antibody, peptide, —CHR′COR, saturated or unsaturated cycloalkyl or heterocycle; wherein said alkyl, alkenyl, alkyldiazo, aryldiazo, alkynyl and saturated or unsaturated cycloalkyl or heterocycle is substituted or unsubstituted;

R is hydrogen, alkyl, alkylamine, —N(Alkyl)₂, OH, alkenyl, alkynyl or saturated or unsaturated cycloalkyl or heterocycle; wherein said alkyl, alkenyl, alkynyl and saturated or cycloalkyl or heterocycle is substituted or unsubstituted; and

R′ is an amino acid side chain.

In one embodiment, this invention is directed to a multinuclear lanthanide chiral cluster comprising phenyl-oxazoline-amide (POxA) ligand or salt thereof represented by the structure of formula IIIA:

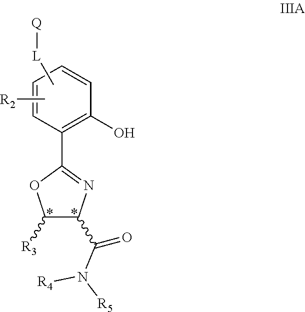

and lanthanide (III) ions;
wherein,
Q is a sensor, monomeric building-block for polymerization, a polymer, chromophore, surface adhesive group or combination thereof;
L is a bond or a linker;

R₂ is hydrogen, alkyl, alkenyl, alkyldiazo, aryldiazo, alkynyl, aryl, halogen, —C≡C-Ph-R, —N═N-Ph-R, CN, NH₂, OH, N₃, NO₂, COOH, COOR, SO₃H, SO₃R, SO₂NHR, O-alkyl, alkylamino, haloalkyl, or R₁ and R₂ combine together to form a 5-7 membered ring; wherein said 5-7 membered ring is saturated or unsatureated cycloalkyl or heterocycle; wherein said alkyl, alkenyl, alkyldiazo, aryldiazo, alkynyl, aryl and saturated or unsaturated cycloalkyl or heterocycle is substituted or unsubstituted;

R₃ is alkyl, aryl, alkenyl, alkyldiazo, aryldiazo, alkynyl, halogen, CN, NH₂, OH, N₃, NO₂, COOH, SO₃H, SO₃R, SO₂NHR, COOR, O-alkyl, alkylamino or haloalkyl; wherein said alkyl, alkenyl, alkyldiazo, aryldiazo, alkynyl and aryl is substituted or unsubstituted;

R₄ is alkyl, alkenyl, alkyldiazo, aryldiazo, alkynyl, polyethylene glycol (PEG), sugars, glucose, manose, galactose, proteins, antibody, peptide, —CHR′COR, saturated or unsaturated cycloalkyl or heterocycle; or R₄ and R₅ combine together with the nitrogen to form a 5-7 membered ring; wherein said 5-7 membered ring is saturated or unsaturated cycloalkyl or heterocycle; wherein said alkyl, alkenyl, alkyldiazo, aryldiazo, alkynyl and saturated or unsaturated cycloalkyl or heterocycle is substituted or unsubstituted;

R₅ is hydrogen, alkyl, alkenyl or alkynyl or R₅ and R₄ combine together with the nitrogen to form a 5-7 membered ring; wherein said 5-7 membered ring is saturated or unsaturated cycloalkyl or heterocycle;

R is hydrogen, alkyl, alkylamine, -N(Alkyl)₂, OH, alkenyl, alkynyl or saturated or unsaturated cycloalkyl or heterocycle; wherein said alkyl, alkenyl, alkynyl and saturated or cycloalkyl or heterocycle is substituted or unsubstituted; and

R′ is an amino acid side chain.

In one embodiment, this invention is directed to a multinuclear lanthanides chiral cluster comprising phenyl-oxazoline-amide (POxA) ligand or salt thereof wherein lanthanide ions coordinate to said POxA ligand as presented by the structure of formula IVa and the structure of formula IVb:

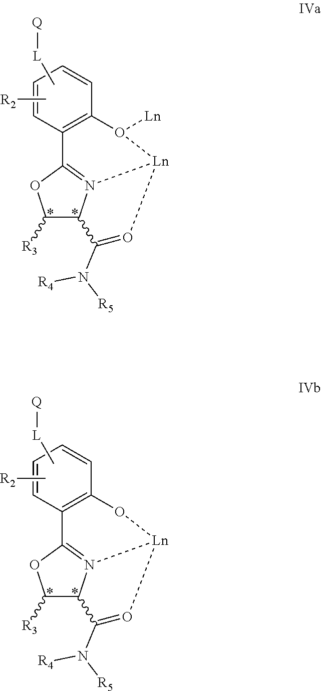

wherein,
Ln is a lanthanide(III) ion;
oxygen bridges coordinate between said lanthanide ions; and said cluster further comprises one or more oxygen based ligands, one or more halogens, or combination thereof;

Q is a sensor, monomeric building-block for polymerization, a polymer, chromophore, surface adhesive group or combination thereof;

L is a bond or a linker;

R₂ is hydrogen, alkyl, alkenyl, alkyldiazo, aryldiazo, alkynyl, aryl, halogen, —C≡C-Ph-R, —N═N-Ph-R, CN, NH₂, OH, N₃, NO₂, COOH, COOR, SO₃H, SO₃R, SO₂NHR, O-alkyl, alkylamino, haloalkyl, or R₁ and R₂ combine together to form a 5-7 membered ring; wherein said 5-7 membered ring is saturated or unsatureated cycloalkyl or heterocycle; wherein said alkyl, alkenyl, alkyldiazo, aryldiazo, alkynyl, aryl and saturated or unsaturated cycloalkyl or heterocycle is substituted or unsubstituted;

R₃ is alkyl, aryl, alkenyl, alkyldiazo, aryldiazo, alkynyl, halogen, CN, NH₂, OH, N₃, NO₂, COOH, SO₃H, SO₃R, SO₂NHR, COOR, O-alkyl, alkylamino or haloalkyl; wherein said alkyl, alkenyl, alkyldiazo, aryldiazo, alkynyl and aryl is substituted or unsubstituted;

R₄ is alkyl, alkenyl, alkyldiazo, aryldiazo, alkynyl, polyethylene glycol (PEG), sugars, glucose, manose, galactose, proteins, antibody, peptide, —CHR′COR, saturated or unsaturated cycloalkyl or heterocycle; or R₄ and R₅ combine together with the nitrogen to form a 5-7 membered ring; wherein said 5-7 membered ring is saturated or unsaturated cycloalkyl or heterocycle; wherein said alkyl, alkenyl, alkyldiazo, aryldiazo, alkynyl and saturated or unsaturated cycloalkyl or heterocycle is substituted or unsubstituted;

R₅ is hydrogen, alkyl, alkenyl or alkynyl or R₅ and R₄ combine together with the nitrogen to form a 5-7 membered ring; wherein said 5-7 membered ring is saturated or unsaturated cycloalkyl or heterocycle

R is hydrogen, alkyl, alkylamine, —N(Alkyl)₂, OH, alkenyl, alkynyl or saturated or unsaturated cycloalkyl or heterocycle; wherein said alkyl, alkenyl, alkynyl and saturated or cycloalkyl or heterocycle is substituted or unsubstituted;

R′ is an amino acidside chain; and

wherein said cluster further comprises one or more oxygen based ligand, one or more halogens or combination thereof.

In another embodiment, the lanthanide ions of the 7Ln clusters further coordinate to said POxA ligand as presented the structure of formula IVc:

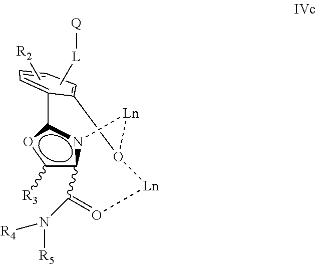

wherein, R₂, R₃, R₄, R₅, L and Q are as described for the structure of formula IIIA; and Ln is a Ln(III) ion.

In one embodiment, the lanthanide ions of the three multinuclear lanthanide cluster coordinate to the POxA ligand of formula IIIA according to structures IVa and IVb in equal ratios.

In one embodiment, the lanthanide ions of the seven multinuclear lanthanide cluster coordinate to the POxA ligand of formula IIIA according to structures IVa, IVb and IVc in equal ratios.

In one embodiment, this invention provides a multinuclear lanthanide chiral cluster comprising a phenyl-oxazoline-amide(POxA) ligand represented by the structure of formula I or IA and a lanthanide ion. In another embodiment, R₁ of the POxA ligand of formula I or IA and/or R₁ of the cluster of formula IIa/IIb/IIc or combination thereof is hydrogen, alkyl, alkenyl, alkyldiazo, aryldiazo, alkynyl, aryl, halogen, —C≡C-Ph-R, —N═N-Ph-R, CN, NH₂, OH, N₃, NO₂, COOH, COOR, SO₃H, SO₃R, SO₂NHR, O-alkyl, alkylamino, haloalkyl, or R₁ and R₂ combine together to form a 5-7 membered ring; wherein said 5-7 membered ring is saturated or unsaturated cycloalkyl or heterocycle; wherein said alkyl, alkenyl, alkyldiazo, aryldiazo, alkynyl, aryl and saturated or unsaturated cycloalkyl or heterocycle is substituted or unsubstituted. In another embodiment, R₁ is hydrogen. In another embodiment, R₁ is halogen. In another embodiment, R₁ is iodo. In another embodiment, R₁ is chloro. In another embodiment, R₁ is bromo. In another embodiment, R₁ is fluoro. In another embodiment, R₁ is —C≡C. In another embodiment, R₁ is SO₃H. In another embodiment, R₁ is SO₃R. In another embodiment, R₁ is SO₂NHR. In another embodiment, R₁ is SO₃Na. In another embodiment, R₁ is NH₂. In another embodiment, R₁ is NO₂. In another embodiment, R₁ is OH. In another embodiment, R₁ is alkyldiazo. In another embodiment, R₁ is OH. In another embodiment, R₁ is —C≡C-Ph-R. In another embodiment, R₁ is OH. In another embodiment, R₁ is —N═N-Ph-N(CH₃)₂. In another embodiment, R₁ is aryldiazo. In another embodiment, R₁ is O-alkyl. In another embodiment, R₁ is H, halogen, alkyldiazo, aryldiazo, —C≡C, SO₃H, SO₃R, SO₂NHR, SO₃Na, —C≡C-Ph-N(CH₃)₂, —N═N-Ph-N(CH₃)₂, NH₂ or NO₂. In another embodiment R₁ is in the para position. In another embodiment R₁ is on the meta position.

In one embodiment, this invention provides a chiral cluster comprising a phenyl-oxazoline-amide (POxA) ligand represented by the structure of formula III or IIIA and a lanthanide ion. In another embodiment, Q of the POxA ligand of formula III or IIIA and/or Q of the cluster of formula IVa/IVb/IVc or combination thereof is a sensor. In another embodiment, the sensor is a molecular sensor. In another embodiment, the sensor comprises a chelator for cation sensing; non limiting examples of cation chelators include bidentate ligands, bipyridyl, 8-hydroxyquinoline, hydroxamates, EDTA or crown ethers. In another embodiment, the molecular sensor comprise bipyridyl for Ru(II) and Cr(III), 8-hydroxyquinolines for Al(III) binding and hydroxamate for iron(III) and Cu(II). In another embodiment, the sensor comprises metalloporphyrins for anion binding and atmospheric gases. In another embodiment, the sensor comprises boronic acid for sugars and amino acid sensing. In another embodiment, the sensor comprises metal phthalocyanine or carbon nanotubes for gas sensing (NO₂, NO, CO, O₂). In another embodiment, the sensor comprises binuclear Zn(II)-dipicolylamine (Dpa) for phosphate sensing. In another embodiment, the sensor is an antibody for a specific antigen. In another embodiment, the sensor comprises glucosamine for glucose. In another embodiment, the sensor comprises hyaluronic acid for CD44 receptor for cancer detection. In another embodiment, the sensor comprises testosterone targeting androgen receptor for ovary & testicle cancer detection. In another embodiment, the sensor comprises antibodies developed to MMP-9 receptors for inflammation detection. In another embodiment, the sensor comprises RGD (Arg-Gly-Asp) for integrin receptors.

In another embodiment the Q is a conductive polymer such as poly(phenylenevinylene) (PPV), Polythiophenes (PTs), and Polypyrrole (PPy). In another embodiment, Q is a polymer such as polyethylene glycol (PEG).

In another embodiment, Q is a monomeric-building-block for self-polymerization. Head-to-head and head-to-tail polymerization. In another embodiment, non limiting examples of monomeric building blocks include alkene or alkyne.

In another embodiment, Q is a conjugated chromophore including polyaromatic groups (naphthalene, anthracene, pyrene, perylene, etc.), diazo dyes with a —N═N— azo structure, and conjugated porphyrins.

The term “sensor” of this invention refers to the cluster of this invention comprising the POxA ligand of formula III or IIIA and a lanthanide ion and/or cluster of formula IVa/IVb/IVc or combination thereof, wherein Q is a molecular sensor. The molecular sensor interacts with a target in a highly selective way, recognize it and as a result the cluster yield an optical (modified luminescence) or a magnetic signal that can be analyzed, and thereby identifying and quantifying the target. In another embodiment Q is a chromophore. The chromophore can be conjugated or non-conjugated to the lanthanide ion. Incorporating a non-conjugated chromophore, with excitation wavelength unlike those of the POxA complex, allow excitations of the embedded lanthanide at two distinct wavelengths. Clusters constructed from such ligands could sensitize different lanthanides to emit, depending on the lanthanide metal, in the visible and the near infrared (NIR) range. Conjugated systems shift the ligand optical properties to the red thus increase the likelihood for clusters emitting in the near infra-red (NIR) region. Examples of conjugated chromophores are: polyaromatic (naphthalene, anthracene, pyrene, etc.), diazo dyes with a —N═N— azo structure, conjugated porphyrin, electron-donors and electron acceptors within the same chromophore.

In one embodiment, the cluster of this invention comprising a chromphore (i.e Q of formula III, IIIA, IVa/IVb/IVc) having an electron-donor and an electron acceptor groups within the same chromophore, may be used in solar energy conversion, having second-order nonlinear optical (NLO) properties.

In one embodiment, Q of formula III, IIIA, IVa/IVb/IVc comprises a surface adhesive group. Non limiting examples of surface adhesive groups include thiol, phosphonate, phosphate, hydroxamate or silyl grups. In another embodiment, the surface adhesive groups are attached to a polymeric chain or attached to a saturated or unsaturated alkyl (C₅₋₂₀) chain.

In one embodiment, this invention provides a chiral cluster comprising a phenyl-oxazoline-amide ligand represented by the structure of formula III, IIIA and a lanthanide ion and/or a cluster of formula IVa/IVb/IVc or combination thereof. In another embodiment, L of the phenyl-oxazoline-amide ligand represented by the structure of formula III, IIIA, IVa/IVb/IVc is a bond. In another embodiment, L is a linker. In another embodiment, the linker is a substituted or unsubstituted: alkyl, alkenyl, alkynyl, alkoxy, amide, triazole, alkyl ether, oxo (C═O), amine, oxygen, amino acid, sulfonamide, or —NH-alkyl-O—.

In one embodiment, this invention provides a chiral cluster comprising a phenyl-oxazoline (POx)-amide ligand of formula I, IA, III or IIIA and a lanthanide ion, and a cluster of formula IIa/IIb/IIc or combination thereof or IVa/IVb/IVc or combination thereof wherein R₂ is hydrogen, alkyl, alkenyl, alkyldiazo, aryldiazo, alkynyl, aryl, halogen, CN, NH₂, OH, N₃, NO₂, COOH, COOR, SO₃H, SO₃R, SO₂NHR, O-alkyl, alkylamino, haloalkyl, or R₁ and R₂ combine together to form a 5-7 membered ring; wherein said 5-7 membered ring is saturated or unsatureated cycloalkyl or heterocycle; wherein said alkyl, alkenyl, alkyldiazo, aryldiazo, alkynyl aryl, and saturated or unsaturated cycloalkyl or heterocycle is substituted or unsubstituted. In another embodiment, R₂ is hydrogen. In another embodiment, R₂ is an alkyl. In another embodiment, R₂ is an alkenyl. In another embodiment, R₂ is alkynyl. In another embodiment, R₂ is an aryl. In another embodiment, R₂ is halogen. In another embodiment, R₂ is CN. In another embodiment, R₂ is NH₂. In another embodiment, R₂ is OH. In another embodiment, R₂ is N₃. In another embodiment, R₂ is NO₂. In another embodiment, R₂ is COOH. In another embodiment, R₂ is alkyldiazo. In another embodiment, R₂ is aryldiazo. In another embodiment, R₂ is COOR. In another embodiment, R₂ is SO₃H. In another embodiment, R₂ is SO₃R. In another embodiment, R₂ is SO₂NHR. In another embodiment, R₂ is O-alkyl. In another embodiment, R₂ is alkylamino. In another embodiment, R₂ is haloalkyl. In another embodiment, R₂ is on the para position. In another embodiment, R₂ is on the ortho position. In another embodiment, R₂ is on the meta position.

In one embodiment, this invention provides a cluster comprising a phenyl-oxazoline (POx)-amide ligand of formula I, IA, III or IIIA and a lanthanide ion, and a cluster of formula IIa/IIb/IIc or combination thereof or IVa/IVb/IVc or combination thereof wherein R₁ and R₂ combine together to form a 5-7 membered ring; wherein said 5-7 membered ring is saturated or unsatureated cycloalkyl or heterocycle. In another embodiment, R₁ and R₂ combine to form a 5 membered ring. In another embodiment, R₁ and R₂ combine to form a 6 membered ring. In another embodiment, R₁ and R₂ combine to form a 7 membered ring. In another embodiment, R₁ and R₂ combine to form phenyl. In another embodiment, R₁ and R₂ combine to form pyridyl. In another embodiment, R₁ and R₂ combine to form cyclohexane. In another embodiment, R₁ and R₂ combine to form dihydrofuran. In another embodiment, R₁ and R₂ combine to form dihydrothiophene. In another embodiment, R₁ and R₂ combine to form thiophene. In another embodiment, R₁ and R₂ combine to form cyclohexane indole. In another embodiment, R₁ and R₂ combine to form dihydroindole.

In one embodiment, this invention provides a chiral cluster comprising a phenyl-oxazoline (POx)-amide ligand of formula I, IA, III or IIIA and a lanthanide ion, and a cluster of formula IIa/IIb/IIc or combination thereof or IVa/IVb/IVc or combination thereof wherein R₃ is alkyl, aryl, alkenyl, alkyldiazo, aryldiazo, alkynyl, halogen, CN, NH₂, OH, N₃, NO₂, COOH, SO₃H, SO₃R, SO₂NHR, COOR, O-alkyl, alkylamino or haloalkyl; wherein said alkyl, alkenyl, alkyldiazo, aryldiazo, alkynyl and aryl is substituted or unsubstituted. In another embodiment, R₃ is an alkyl. In another embodiment, R₃ is an alkenyl. In another embodiment, R₃ is alkynyl. In another embodiment, R₃ is an aryl. In another embodiment, R₃ is halogen. In another embodiment, R₃ is CN. In another embodiment, R₃ is NH₂. In another embodiment, R₃ is alkyldiazo. In another embodiment, R₃ is aryldiazo. In another embodiment, R₃ is OH. In another embodiment, R₃ is N₃. In another embodiment, R₃ is NO₂. In another embodiment, R₃ is COOH. In another embodiment, R₃ is COOR. In another embodiment, R₃ is SO₃H. In another embodiment, R₃ is SO₃R. In another embodiment, R₃ is SO₂NHR. In another embodiment, R₃ is O-alkyl. In another embodiment, R₃ is methyl. In another embodiment, R₃ is alkylamino. In another embodiment, R₃ is haloalkyl.

In one embodiment, this invention provides a chiral cluster comprising a phenyl-oxazoline (POx)-amide ligand of formula I, IA, III or IIIA and a lanthanide ion, and a cluster of formula IIa/IIb/IIc or combination thereof or IVa/IVb/IVc or combination thereof wherein R₄ is alkyl, alkenyl, alkyldiazo, aryldiazo, alkynyl, polyethylene glycol (PEG), sugars, glucose, manose, galactose, proteins, antibody, peptide, —CHR′COR, saturated or unsaturated cycloalkyl or heterocycle; or R₄ and R₅ combine together with the nitrogen to form a 5-7 membered ring; wherein said 5-7 membered ring is saturated or unsaturated cycloalkyl or heterocycle; wherein said alkyl, alkenyl, alkyldiazo, aryldiazo, alkynyl and saturated or unsaturated cycloalkyl or heterocycle is substituted or unsubstituted. In another embodiment, R₄ is alkyl. In another embodiment, R₄ is alkenyl. In another embodiment, R₄ is alkyldiazo. In another embodiment, R₄ is aryldiazo. In another embodiment, R₄ is alkynyl. In another embodiment, R₄ is polyethylene glycol (PEG). In another embodiment, R₄ is a sugar. In another embodiment, R₄ is glucose. In another embodiment, R₄ is manose. In another embodiment, R₄ is galactose. In another embodiment, R₄ is a protein. In another embodiment, R₄ is an antibody. In another embodiment, R₄ is a peptide. In another embodiment, R₄ is —CHR′COR. In another embodiment, R₄ is saturated or unsaturated cycloalkyl or heterocycle. In another embodiment R₄ is alkyl or saturated or unsaturated cycloalkyl or heterocycle.

In one embodiment, R₄ of formula I, IA, IIa, IIb, IIc, III, IIA, IVa, IVb, IVc is CHR′COR wherein R′ is an amino acid side chain. In another embodiment, R′ is a side chain of a natural or unnatural amino acid. Non limiting examples of R′ include hydrogen (side chain of glycine), CH₃ (side chain of alanine), CH₂OH (serine), CH₂SH (cysteine), —CH(OH)CH₃ (threonine), —CH(CH₃)₂ (valine), —CH₂CH(CH₃)₂ (leucine), CH₂COOH (aspartic acid), CH₂CH₂COOH (glutamic acid), —(CH₂)₄NH₂ (lysine), —CH₂Ph (phenylalanine) or —CH₂PhOH (tyrosine).

In one embodiment, this invention provides a chiral cluster comprising a phenyl-oxazoline (POx)-amide ligand of formula IA, IIIA and a lanthanide ion, and a cluster of formula IIa/IIb/IIc or combination thereof or IVa/IVb/IVc or combination thereof wherein R₅ is hydrogen, alkyl, alkenyl or alkynyl or R₅ and R₄ combine together with the nitrogen to form a 5-7 membered ring; wherein said 5-7 membered ring is saturated or unsaturated cycloalkyl. In another embodiment, R₅ is hydrogen. In another embodiment, R₅ is alkyl. In another embodiment, R₅ is alkenyl. In another embodiment, R₅ is alkynyl.

In another embodiment R₅ and R₄ of the structure of formula IA and IIIA and the clusters of formula IIa/IIb/IIc or IVa/IVb/IVc combine together with the nitrogen to form a 5-7 membered ring; wherein said 5-7 membered ring is saturated or unsaturated cycloalkyl. In another embodiment, R₄ and R₅ combine to form a 5 membered ring. In another embodiment, R₄ and R₅ combine to form a 6 membered ring. In another embodiment, R₄ and R₅ combine to form a 7 membered ring. In another embodiment, R₄ and R₅ combine to form morpholine. In another embodiment, R₄ and R₅ combine to form morpholine, piperidine, pyridine, thiazole, imidazole, oxazole, pyrrole or pyrazine.

In one embodiment, this invention provides a chiral cluster comprising a phenyl-oxazoline (POx)-amide ligand of formula I, IA, III or IIIA and a lanthanide ion, and a cluster of formula IIa/IIb/IIc or combination thereof or IVa/IVb/IVc or combination thereof wherein R is hydrogen, alkyl, alkylamine, —N(Alkyl)₂, OH, alkenyl, alkynyl or saturated or unsaturated cycloalkyl or heterocycle; wherein said alkyl, alkenyl, alkynyl and saturated or cycloalkyl or heterocycle is substituted or unsubstituted. In another embodiment, R is hydrogen. In another embodiment, R is alkyl. In another embodiment, R is alkenyl. In another embodiment, R is alkynyl. In another embodiment, R is alkylamine. n another embodiment, R is —N(Alkyl)₂. In another embodiment, R is alkyl saturated or unsaturated cycloalkyl. In another embodiment, R is saturated or unsaturated heterocycle.

In one embodiment, the clusters of this invention include lanthanides wherein the lanthanide is La(III), Ce(III), Pr(III), Nd(III), Pm(III), Sm(III), Eu(III), Gd(III), Tb(III), Dy(III), Ho(III), Er(III), Tm(III), Yb(III) or Lu(III). In another embodiment, the cluster includes 3 lanthanides. In another embodiment, the cluster includes 7 lanthanides. In another embodiment, the cluster includes 5 lanthanides. In another embodiment, the cluster includes between 3-10 lanthanides. In another embodiment, the lanthanide is La(III). In another embodiment, the lanthanide is Pr(III). In another embodiment, the lanthanide is Nd(III). In another embodiment, the lanthanide is Pm(III). In another embodiment, the lanthanide is Sm(III). In another embodiment, the lanthanide is Eu(III). In another embodiment, the lanthanide is Gd(III). In another embodiment, the lanthanide is Tb(III). In another embodiment, the lanthanide is Dy(III). In another embodiment, the lanthanide is Ho(III). In another embodiment, the lanthanide is Er(III). In another embodiment, the lanthanide is Tm(III). In another embodiment, the lanthanide is Yb(III). In another embodiment, the lanthanide is or Lu(III). In another embodiment, the lanthanides are the same. In another embodiment, the lanthanides are different.

The term “alkyl” in this invention refers both to linear and to branched alkyl. In one embodiment, the term “alkyl” refers to a saturated linear aliphatic hydrocarbon chain. In another embodiment, the term “alkyl” refers to a saturated branched aliphatic hydrocarbon chain. In one embodiment, the alkyl group has 1-12 carbons. In another embodiment, the alkyl group has 2-8 carbons. In another embodiment, the alkyl group has 1-6 carbons. In another embodiment, the alkyl group has 1-4 carbons. In another embodiment, the branched alkyl is an alkyl substituted by alkyl side chains of 1 to 5 carbons. In another embodiment, the branched alkyl is an alkyl substituted by haloalkyl side chains of 1 to 5 carbons. The alkyl group may be unsubstituted or substituted, wherein said substitutions include but are not limited to: halogen, alkyl of 1 to 6 carbons, alkoxy of 1 to 6 carbons, ester of 1 to 6 carbons, carboxy, cyano, nitro, hydroxyl, thiol, amine, amide, reverse amide, sulfonamide, phosphate, aryl, phenyl or any combination thereof.

The alkyl group can be a sole substituent or it can be a component of a larger substituent, such as in an alkoxy, haloalkyl, arylalkyl, alkylamino, etc. Preferred alkyl groups are methyl, ethyl, and propyl, and thus halomethyl, dihalomethyl, trihalomethyl, haloethyl, dihaloethyl, trihaloethyl, halopropyl, dihalopropyl, trihalopropyl, methoxy, ethoxy, propoxy, methylamino, ethylamino, propylamino, dimethylamino, diethylamino, methylamido, acetamido, propylamido, etc.

As used herein, the term “aryl” refers to any aromatic ring that is directly bonded to another group and can be either substituted or unsubstituted. The aryl group can be a sole substituent, or the aryl group can be a component of a larger substituent, such as in an arylalkyl, arylamino, arylamido, etc. Exemplary aryl groups include, without limitation, phenyl, tolyl, xylyl, furanyl, naphthyl, pyridinyl, pyrimidinyl, pyridazinyl, pyrazinyl, triazinyl, thiazolyl, oxazolyl, isooxazolyl, pyrazolyl, imidazolyl, thiophene-yl, pyrrolyl, perylene, perylenediimide, naphthylimides, pyrene, phenylmethyl, phenylethyl, phenylamino, phenylamido, etc. Substitutions include but are not limited to: F, Cl, Br, I, C₁-C₅ linear or branched alkyl, C₁-C₅ linear or branched haloalkyl, C₁-C₅ linear or branched alkoxy, C₁-C₅ linear or branched haloalkoxy, CF₃, CN, NO₂, —CH₂CN, NH₂, NH-alkyl, N(alkyl)₂, hydroxyl, —OC(O)CF₃, —OCH₂Ph, —NHCO-alkyl, COOH, —C(O)Ph, C(O)O-alkyl, C(O)H, or —C(O)NH₂.

As used herein, the term “alkoxy” or “—O-alkyl” refers to an ether group substituted by an alkyl group as defined above. Alkoxy refers both to linear and to branched alkoxy groups. Nonlimiting examples of alkoxy groups are methoxy, ethoxy, propoxy, iso-propoxy, tert-butoxy.

As used herein, the term “alkylamino” refers to an alkyl group as defined above substituted by an amine group. Alkylamino refers to alkylamino, alkyldiamino or alkyltriamino. Nonlimiting examples of alkylamino groups are —CH₂NH₂, —CH₂CH₂NH₂, —CH₂CH(NH₂)₂.

A “haloalkyl” group refers, in another embodiment, to an alkyl group as defined above, which is substituted by one or more halogen atoms, e.g. by F, Cl, Br or I. Nonlimiting examples of haloalkyl groups are CF₃, CF₂CF₃, CH₂CF₃.

A “cycloalkyl” or “carbocyclic” group refers, in one embodiment, to a ring structure comprising carbon atoms as ring atoms, which may be either saturated or unsaturated, substituted or unsubstituted. In another embodiment the cycloalkyl is a 3-12 membered ring. In another embodiment the cycloalkyl is a 6 membered ring. In another embodiment the cycloalkyl is a 5-7 membered ring. In another embodiment the cycloalkyl is a 3-8 membered ring. In another embodiment, the cycloalkyl group may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. In another embodiment, the cycloalkyl ring may be fused to another saturated or unsaturated cycloalkyl or heterocyclic 3-8 membered ring. In another embodiment, the carbocycle ring is a saturated ring. In another embodiment, the carbocycle ring is an unsaturated ring. Non limiteing examples of a cycloalkyl or carbocycle group comprise cyclohexyl, cyclohexenyl, cyclopropyl, cyclopropenyl, cyclopentyl, phenyl, cyclopentenyl, cyclobutyl, cyclobutenyl, cycloctyl, cycloctadienyl (COD), cycloctaene (COE) etc.

A “heterocycle” or “heterocycle” group refers, in one embodiment, to a ring structure comprising in addition to carbon atoms, sulfur, oxygen, nitrogen or any combination thereof, as part of the ring. In another embodiment the heterocycle is a 3-12 membered ring. In another embodiment the heterocycle is a 6 membered ring. In another embodiment the heterocycle is a 5-7 membered ring. In another embodiment the heterocycle is a 3-8 membered ring. In another embodiment, the heterocycle group may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. In another embodiment, the heterocycle ring may be fused to another saturated or unsaturated cycloalkyl or heterocyclic 3-8 membered ring. In another embodiment, the heterocyclic ring is a saturated ring. In another embodiment, the heterocyclic ring is an unsaturated ring. Non limiting examples of a heterocyclic rings comprise pyridine, saccharide, piperidine, morpholine, piperazine, thiophene, pyrrole, benzodioxole, or indole. In another embodiment, the heterocycle is a morpholine or a saccharide.

In one embodiment, this invention provides a cluster of this invention or its salt thereof. In another embodiment, a salt of the clusters include alkaline metals such as Li⁺, Na⁺, K⁺; alkaline metals such as Mg²⁺, Ca²⁺; NH₃⁺, Cl⁻, Br⁻, I⁻. In another embodiment, this invention provides purified isomers of the cluster of this invention. In another embodiment, this invention provides a polymorph of the cluster of this invention. In another embodiment, this invention provides a crystal of the cluster of this invention.

In one embodiment, the term “isomer” includes, but is not limited to, optical isomers and analogs, structural isomers and analogs, conformational isomers and analogs, and the like. In another embodiment, the isomer is an optical isomer.

In one embodiment, this invention encompasses the use of various optical isomers of the cluster of the invention. It will be appreciated by those skilled in the art that the ligands of the present invention contain at least two chiral center. Accordingly, the ligands used in the methods of the present invention are in optically-active forms.

In one embodiment, the ligands are the (RR)-stereoisomers. In another embodiment, the ligands are the (SS)-stereoisomers. In another embodiment, the ligands are the (RS)-stereoisomers. In another embodiment, the ligands are the (SR)-stereoisomers.

In one embodiment, the cluster of this invention comprise ligands which are substantially free from its corresponding stereoisomer (i.e. substantially pure). Substantially pure, refers to a stereoisomer which is at least about 95% pure from its corresponding stereoisomer, more preferably at least about 98% pure from its corresponding stereoisomer, most preferably at least about 99% pure from its corresponding stereoisomer. The ligands of this invention are prepared from optically-active starting materials.

In one embodiment, the phenyl-oxazoline-amide ligand is prepared by cyclization of a threonine precursor using SOCl₂.

In another embodiment, the clusters of this invention are prepared by mixing the phenyl-oxazoline-amide of this invention with LiOH and subsequent addition of LnCl₃.

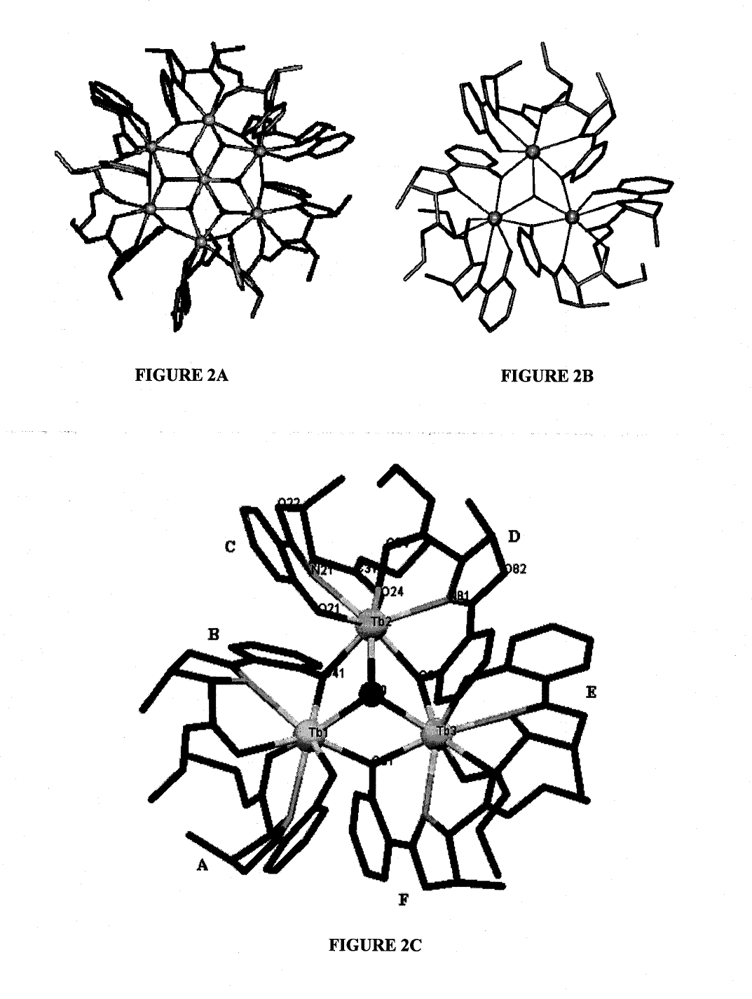

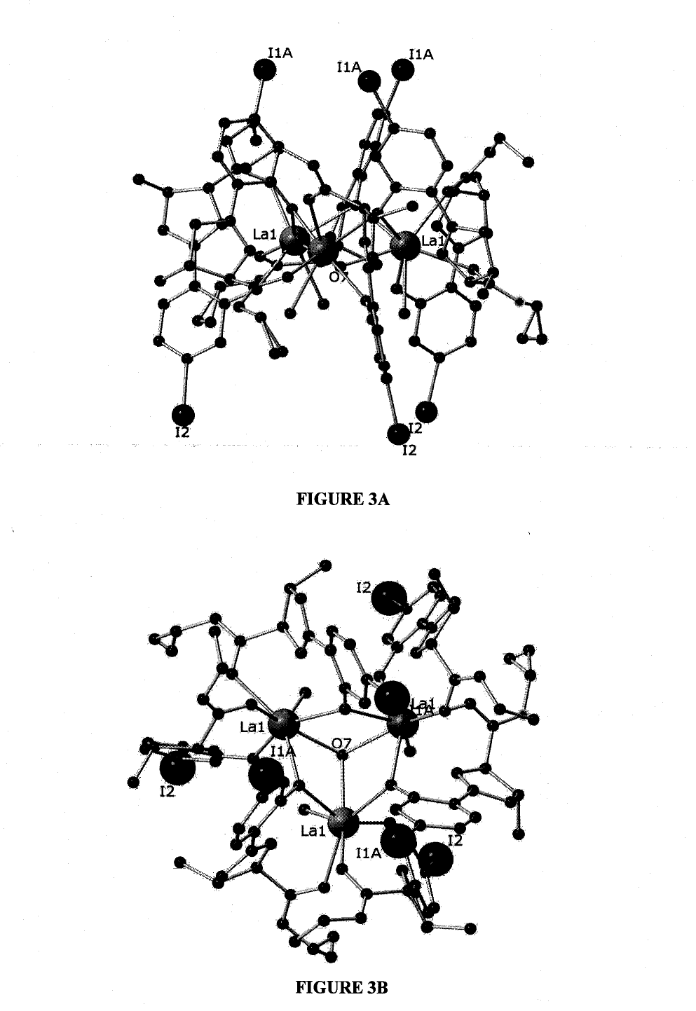

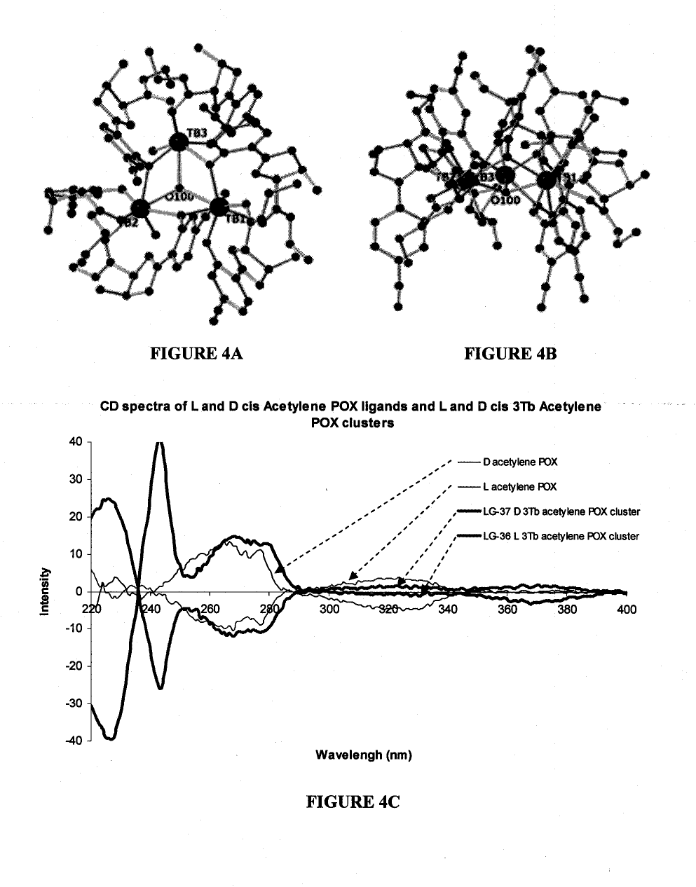

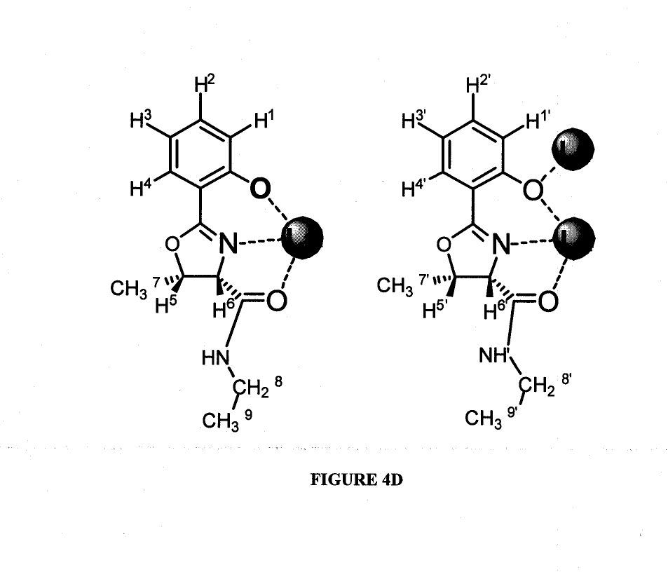

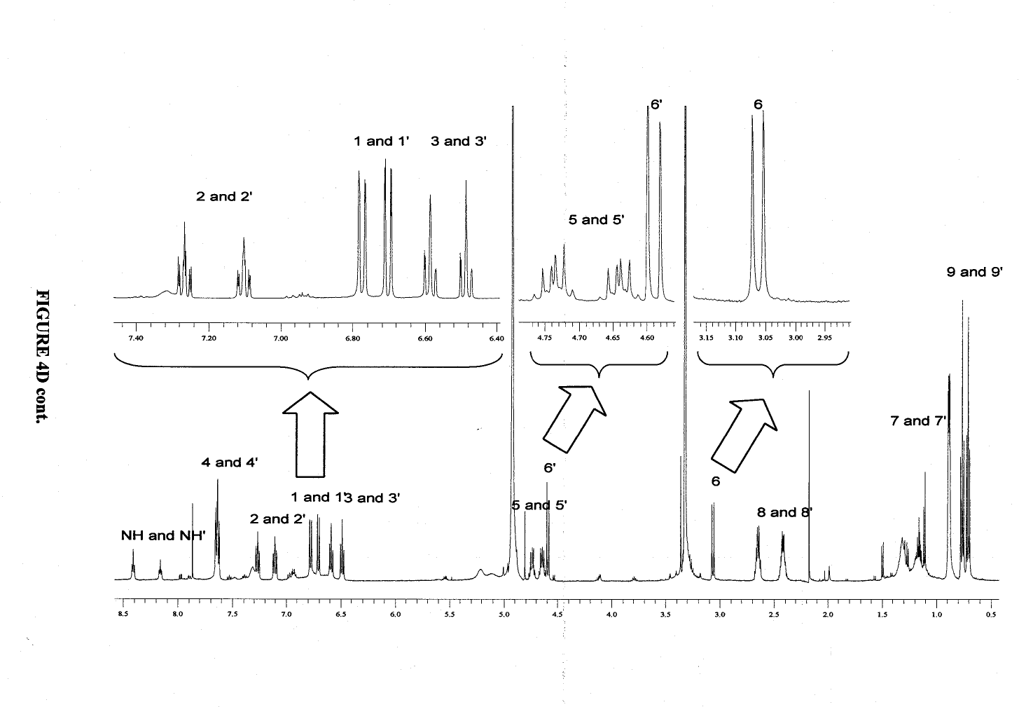

In one embodiment, the chiral cluster of this invention is a three lanthanide (3Ln) cluster wherein the phenyl-oxazoline-amide ligand is a cis isomer. The cis isomer of the phenyl-oxazoline-amide ligand includes 4R, 5R or 4S,5S chiral centers. In one embodiment, a 3Ln cluster is prepared according to Example 20. In one embodiment, this invention provides a crystalline 3Ln cluster as presented in FIG. 2B, FIG. 2C, FIG. 3 and in FIG. 4.

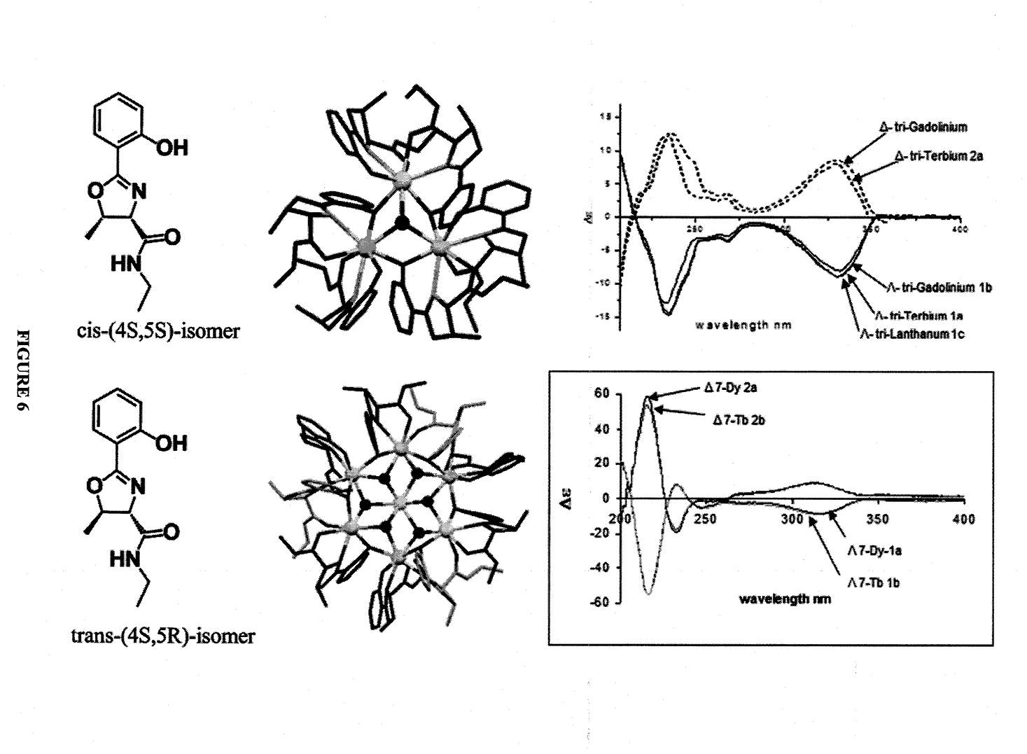

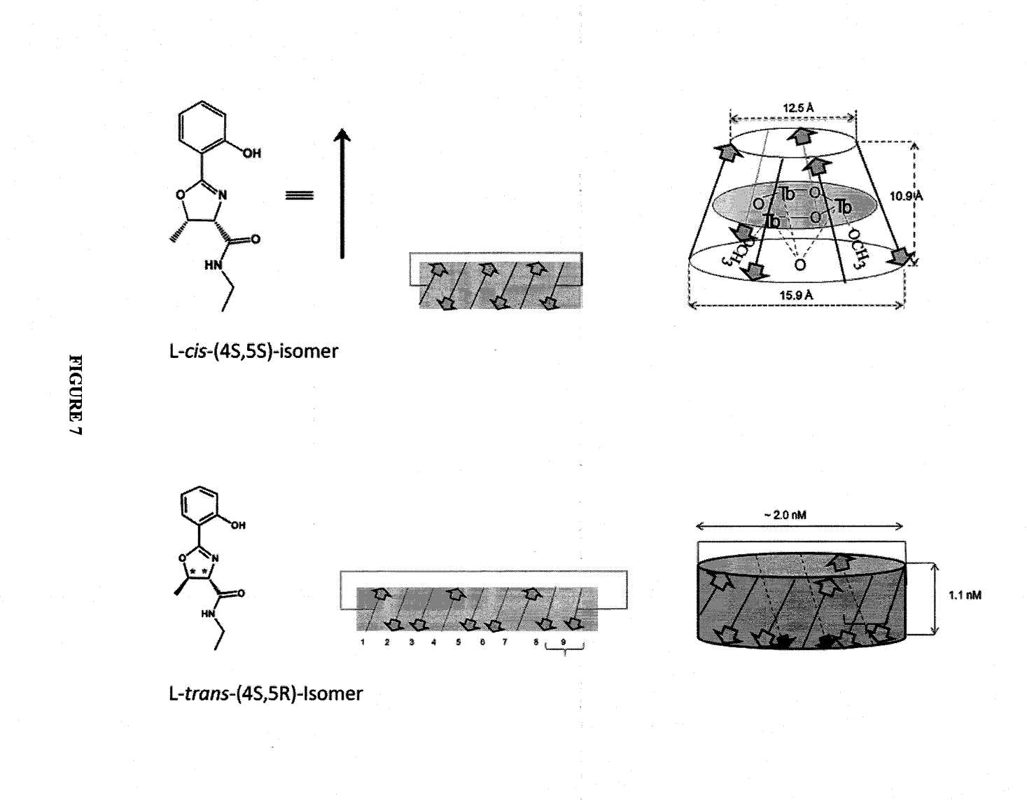

In one embodiment, the chiral cluster of this invention is a seven lanthanide (7Ln) cluster wherein the phenyl-oxazoline-amide ligand is a trans isomer. The trans isomer of the phenyl-oxazoline-amide ligand includes 4R,5S or 4S,5R chiral centers. In one embodiment, a 7Ln cluster is prepared according to Example 20. In one embodiment, this invention provides crystalline structure of a 7Ln cluster as presented in FIG. 2A and in FIG. 6.

In one embodiment, the chiral cluster of this invention include the trans 4R, 5S or trans 4S,5R or cis 4S,5S or cis 4RS,5R phenyl-oxazoline-amide ligand. In another embodiment, the cluster of this invention is circularly polarized. In another embodiment, the cluster of this invention emits circularly polarize luminescence (CPL).

The term “cluster” of this invention refers to an array of phenyl-oxazoline amide ligands, coordinated to lanthanide ions and optionally to, halogens, of oxygen based ligand, wherein the phenyl-oxazoline amide ligands are not covalently linked to each other.

In one embodiment, the cluster of this invention includes 3 lanthanides (Ln(III)) and 6 cis phenyl-oxazoline-amide (POxA) ligands of this invention and one or more oxygen based ligands, one or more halogens, or combination thereof. In another embodiment, the coordination of the lanthanide is 8. In another embodiment the 3Ln cluster includes 3 oxygen based ligands and/or halogens.

In one embodiment, the cluster of this invention includes 7 lanthanides (Ln(III)) and 9 trans-phenyl-oxazoline-amide (POxA) ligands of this invention and 4 oxygen based ligands, and/or halogens, or combination thereof. In another embodiment, the coordination of the lanthanide is 8, with the central Ln with a coordination of 7.

In another embodiment, the oxygen based ligand is alcohol. In another embodiment, the oxygen based ligand is H₂O. In another embodiment, the oxygen based ligand is methanol. In another embodiment, the oxygen based ligand is ethanol. In another embodiment, the oxygen based ligand is isopropanol. In another embodiment, the cluster includes halogen. In another embodiment, the halogen is fluoro. In another embodiment, the halogen is chloro. In another embodiment, the halogen is iodo. In another embodiment, the halogen is bromo.

In one embodiment, an oxygen bridges between the lanthanides. Introducing multiple oxygen bridges between the metal centers greatly improves the stability of the cluster.

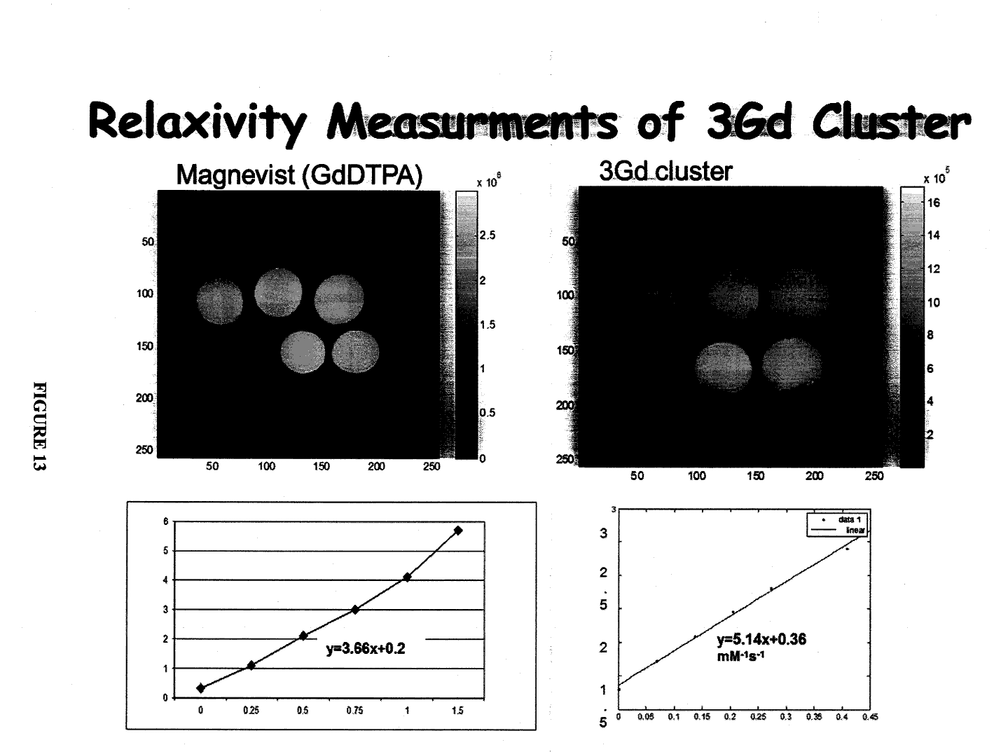

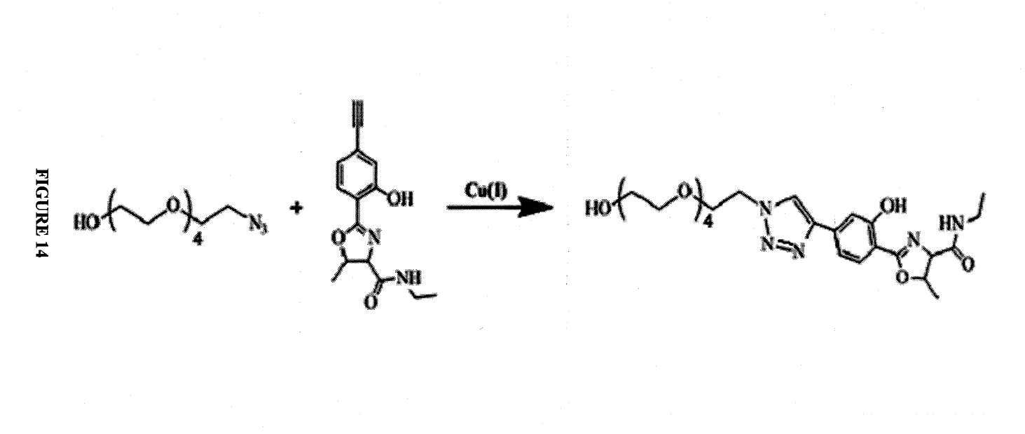

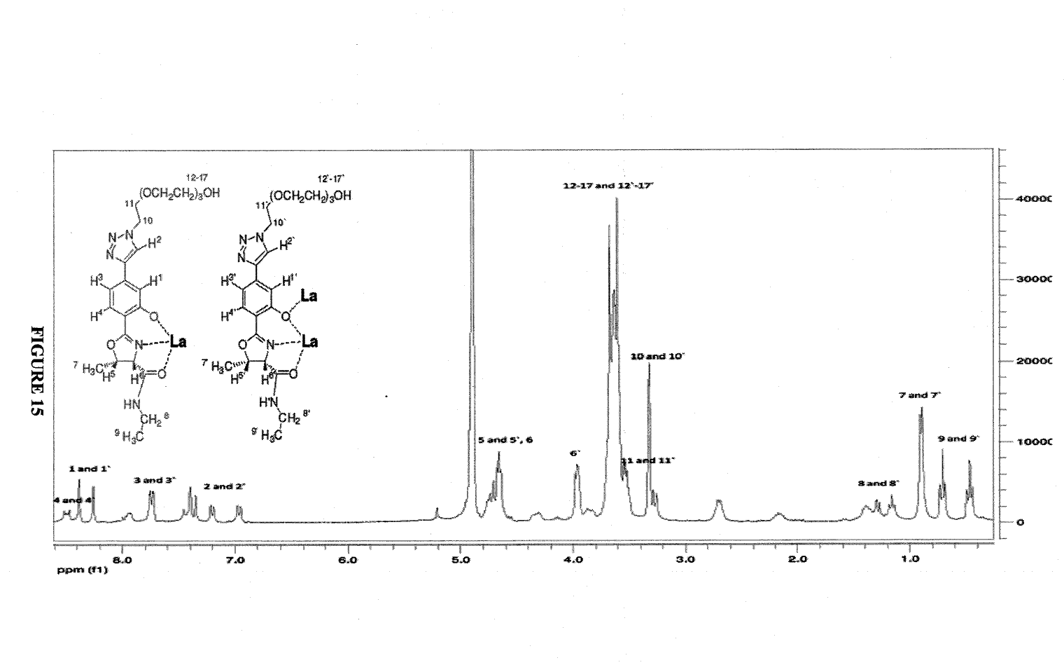

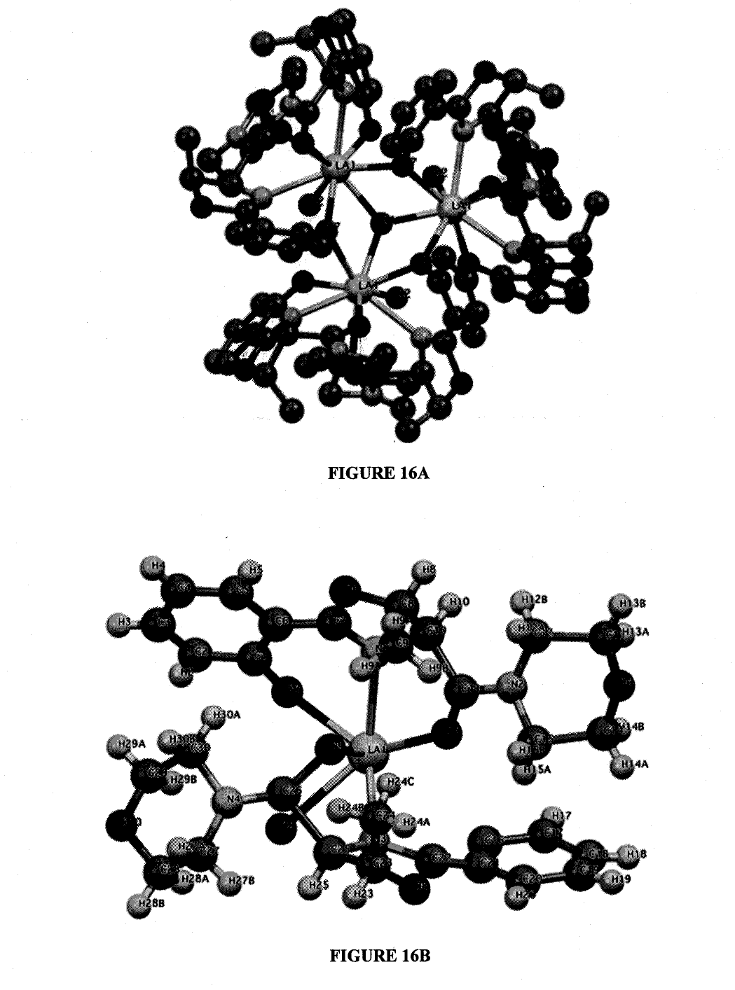

In one embodiment, the chiral cluster of this invention possesses high magnetic properties. In another embodiment, the magnetic properties of the chiral clusters of this invention are as presented in FIG. 13. In another embodiment, the magnetic properties of the chiral clusters of this invention are as presented in FIG. 14. In another embodiment the 3Ln clusters of this invention possess electron magnetic dipole moment of between 10-20 Bohr magneton. In another embodiment the 7Ln clusters of this invention possess electron magnetic dipole moment of between 20-30 Bohr magneton.

In one embodiment, this invention is directed to chiral phenyl-oxazoline ligands and methods of use thereof for magnet technology including (i) magnetic field crystals; (ii) magnetic refrigeration; and (iii) contrast agents in MRI.

In one embodiment, this invention is directed to chiral phenyl-oxazoline ligands and methods of use thereof for (i) emitters in color display devices; (ii) dyes/inks in document and product authenticity; (iii) information, transfer in optical fibers; (iv) biomarkers; (v) electroluminescent materials; (vi) luminescent bio-probes; (vii) ‘markers’ in encoding inks; (viii) NMR shift reagents; (ix) contrast agents in magnetic resonance imaging (MRI); (imaging is possible either by direct visualization or time-resolved spectroscopy; (x) organ-specific-carriers for radioactive lanthanide isotopes; and (xi) single molecule magnets (SMM).

The clusters of this invention provides (i) sharp multi-peak luminescence spectra in the visible and the NIR spectral region; (ii) greatly amplified luminescence, by several orders of magnitude with respect to mono-lanthanide complexes obtained from the same ligand system; (iii) the emitted luminescence from all clusters is circularly polarized. (originating from anisotropic nature of chiral ligands); (iv) luminescence is observed both in the solid and in solution; and (v) luminescence takes place in aqueous solution as well. In one embodiment, the clusters of this invention are embedded into sol-gel matrix or copolymerized with Poly(methyl methacrylate) (PMMA) resulting in transparent materials with mechanical and atmospheric stability and maintaining its optical properties.

In one embodiment, this invention provides an electroluminescent material comprising a chiral cluster comprising a phenyl-oxazoline (POx)-amide ligand of formula III or IIIA and a lanthanide ion, and a cluster of formula IVa/IVb/IVc or combination thereof wherein Q is a conductive polymer. In another embodiment, this invention provides a device comprising an electroluminescent material comprising a chiral cluster comprising a phenyl-oxazoline (POx)-amide ligand of formula III or IIIA and a lanthanide ion, and a cluster of formula IVa/IVb/IVc or combination thereof wherein Q is a conductive polymer. In another embodiment, this invention provides a LED, OLED, thin-film transistors or photovoltaic devices comprising a chiral cluster comprising a phenyl-oxazoline (POx)-amide ligand of formula III or IIIA and a lanthanide ion, and a cluster of formula IVa/IVb/IVc or combination thereofwherein Q is a conductive polymer. In another embodiment, nonlimiting of conductive polymers are polyacetylene, poly(phenylenevinylene) (PPV), Polythiophenes (PTs), and Polypyrrole (PPy).

In one embodiment, chiral clusters having functional groups for cross-coupling reaction (such as N₃, acetylene, CN, halogen, alkene, alkyne) may serve as monomeric building-blocks for self-polymerization. Head-to-head and head-to-tail polymerization is expected to leads to material with comprehensive CPL properties (helical structures amplify (CPL)). In another embodiment, this invention provides polymerized clusters with electroluminescent properties. In another embodiment, this invention provides a multicolor light emitting diode (LED), organic light emitting diode (OLED), thin-film transistors, for photovoltaic applications comprising the polymerized clusters.

In one embodiment, this invention provides a coating material comprising the chiral cluster comprising a phenyl-oxazoline (POx)-amide ligand of formula III or IIIA and a lanthanide ion, and a cluster of formula IVa/IVb/IVc or combination thereof, wherein Q is a surface adhesive and the surface adhesive comprise a thiol, phosphonate, hydroxamate or silyl groups. In another embodiment, the surface adhesive groups are attached to a polymeric chain or saturated or unsaturated alkyl (C₅₋₂₀) chain. Covalent attachment of surface-adhesive functional groups to the clusters forms coating material that integrate optical properties to the surfaces, since the cluster contain two functionalized faces with a 180° angel between them, some will coat the surface and the other be exposed to solvent or air, ready for interacting with a second surface, or nano-particles, generating monomolecular junctions.

In another embodiment, this invention provides a metal sensor comprising a chiral cluster comprising a phenyl-oxazoline (POx)-amide ligand of formula III or IIIA and a lanthanide ion, and a cluster of formula IVa/IVb/IVc or combination thereof, wherein Q is a metal chelator and upon binding to a metal the luminescent properties of the cluster are changed and thereby identifying and quantifying said metal.

In another embodiment, this invention provides an atmospheric gas sensor comprising a chiral cluster comprising a phenyl-oxazoline (POx)-amide ligand of formula III or IIIA and a lanthanide ion, and a cluster of formula IVa/IVb/IVc or combination thereof, wherein Q is mettaloporphyrin and upon binding to an atmospheric gas the luminescent properties of the cluster are changed and thereby identifying and quantifying said gas.

In one embodiment, this invention provides chiral clusters providing circularly polarize luminescence (CPL). In another embodiment, this invention provides a display device comprising the circularly polarize luminescence clusters of this invention. A device based on the CPL clusters of this invention does not need a polarizer and thereby providing energy conservation and increase in battery life time. This invention provides a 3D display comprising the cluster of this invention. In another embodiment, this invention provides liquid crystals comprising the CPL clusters of this invention. In another embodiment, this invention, provides liquid crystals displays comprising the CPL clusters of this invention. In another embodiment, this invention provides ink-jet printing comprising the CPL clusters of this invention.

In one embodiment, this invention provides an inkjet printing comprising a chiral cluster of this invention. The dyes/inks can be used to mark product authenticity, and/or to overlay on printed materials due to the lanthanide delayed emission properties, example: paper with and without florescent additives; taking advantage of the cluster properties including (i) fast magnetic-reader (ii) measuring VIS and possible NIR emission generally characteristic by several emission signals for each lanthanides (iii) measurement of circular polarized luminescence, by a suitable filter. The lanthanide clusters of this invention form color combination for designated coding.

In one embodiment, this invention provides an optical fiber comprising a chiral cluster of this invention. The chiral cluster of this invention can be used as codes in fiber optics for information transmission. Utilizing the fact that optical signal do not interfere with each others and multiple information channels can be used within a single fiber at the same time.

In one embodiment, this invention provides a method of coding and reading said coded information comprising writing a code with a chiral cluster of this invention, and reading said code by measuring its magnetic properties, its luminescence in visible or NIR or by measuring its emission light for circular polarized luminescence (CPL).

In one embodiment, this invention provides a biomarker comprising chiral cluster comprising a phenyl-oxazoline (POx)-amide ligand of formula III or IIIA and a lanthanide ion, and a cluster of formula IVa/IVb/IVc or combination thereof wherein Q is a sensor. The sensor is covalently linked to the cluster of this invention. The sensor can reach specific tissue and/or cellular targets and thereby provide ‘signaling’ platform to display unique luminescence properties for identifying a recognition event.

In one embodiment, this invention is directed to a method of identifying and quantifying a biomolecule in a sample, comprising
(i) contacting a sample comprising a biomolecule with a chiral cluster of this invention wherein said biomolecule is selected from peptides, proteins, oligonucleotides, nucleic acids, oligosaccharides, polysaccharides, glycoproteins, phospholipids and enzymes; and
(ii) measuring luminescence following interaction between said biomolecule and said chiral cluster;
thereby identifying and quantifying the biomolecule in said sample.

In another embodiment, the fluorescence is measured directly from the lanthanide (III).

In one embodiment, the terms “a” or “an” as used herein, refer to at least one, or multiples of the indicated element, which may be present in any desired order of magnitude, to suit a particular application, as will be appreciated by the skilled artisan. In one embodiment, the term “a ligand” refers to two or more ligands. In another embodiment, “phenyl-oxazoline-amide” ligand is referred as the ligand of the invention or as POx ligand or as POxA ligand. In some embodiments, the chiral clusters of this invention and methods of this invention may comprise and/or make use of multiple kinds of clusters and/or combination of clusters of this invention.

The following examples are presented in order to more fully illustrate the preferred embodiments of the invention. They should in no way, however, be construed as limiting the broad scope of the invention.

### EXAMPLES

### Materials and Methods

All the chemicals were obtained from standard commercial supplies unless otherwise indicated and used without further purification. Terbium(III) chloride hexahydrate was purchased from Sigma-Aldrich. Flash chromatography was performed using Merck 230-400 mesh silica gel. Thin-layer chromatography (TLC) on 60E-254 silica gel was visualized with UV light.

¹H NMR spectra were recorded on Varian VXR 400 MHz (Bruker) using either CDCl₃ or MeOD as a solvent. All J values are given in Hz. UV/V is spectra were recorded on a Hewlett-Packard model 8450A diode array spectrophotometer. Fluorescent spectra were recorded on SLM-AMINCO 8100 Series 2 Spectrofluorometer. CD spectra were recorded on Chirascan Applied Photophysics.

### Example 1

### Synthesis of L-cis (4S, 5S) POxA

### L-cis (4S, 5S) (4S,5S)-N-ethyl-2-(2-hydroxyphenyl)-5-methyl-4,5-dihydrooxazole-4-carboxamide

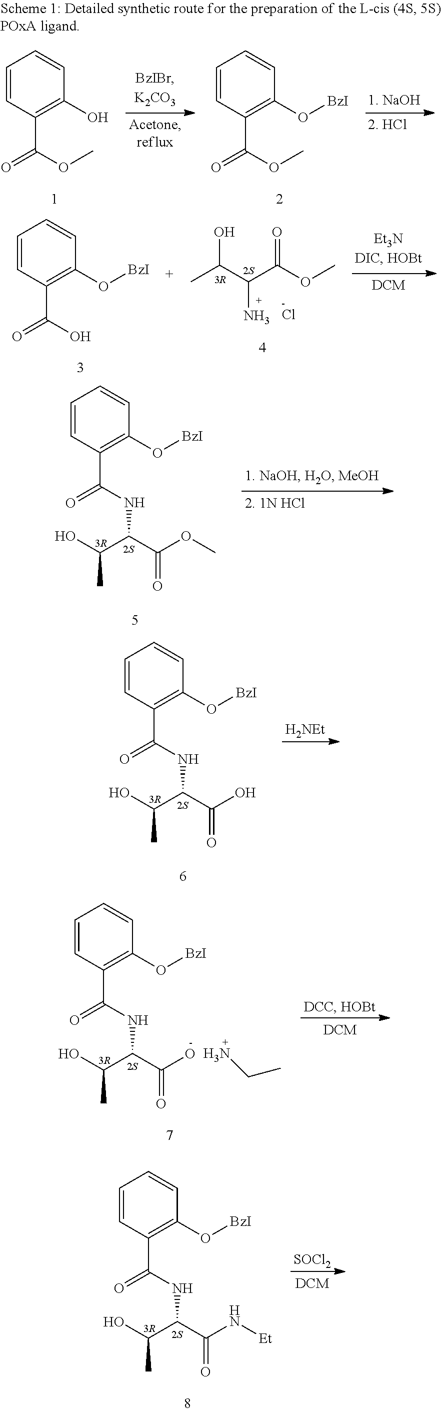

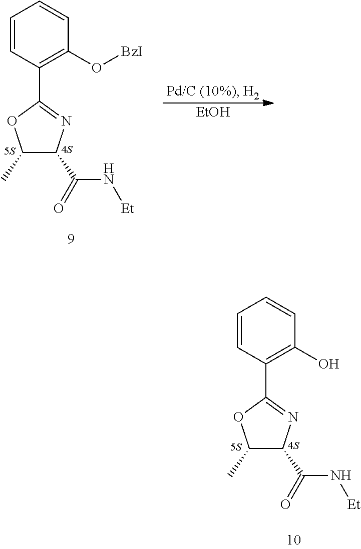

Synthesis of methyl 2-(benzyloxy)benzoate (2): Methyl salicylate (1) (16.17 g, 106 mmol, 13.8 ml) and benzyl bromide (20.25 g, 118.4 mmol, 13.9 ml) were dissolved in acetone (600 ml) and anhydrous K₂CO₃ (52.5 g, 380.4 mmol) was added. The reaction mixture was refluxed for 18 h. the precipitate of potassium carbonate was filtered off and the solvent was evaporated. The residue was dissolved in EA (150 ml) and the organic solution was washed with 1N NaOH (37 ml), water (37 ml) and brine, and dried over Na₂SO₄. Evaporation of the solvent afforded 2 as oil (27.2 g) which was later crystallized (Quantitative yield).

Synthesis of 2-(benzyloxy)benzoic acid (3): Methyl 2-(benzyloxy)benzoate 2 (27 g, 106 mmol) was dissolved in MeOH (600 ml). 5N NaOH (424 mmol) was added with exothermic heating to 40° C. After the reaction mixture was stirred at RT overnight, it was acidified with a 5.5 N HCl solution (80 ml) (solution became clear) and concentrated in vacuum to form precipitate which was extracted with EA (300 ml). The organic layer was washed with water (45 ml) and brine (45 ml) and dried over Na₂SO₄. The solvent was evaporated to obtain 3 as solid (23.7 g). Yield: 98.2%.

Synthesis of (2S,3R)-methyl 2-(2-(benzyloxy)benzamido)-3-hydroxybutanoate (5): L-Threonine methyl ester•HCl ((2S,3R)-methyl 2-amino-3-hydroxybutanoate), 4, (6.27 g, 41.14 mmol) was dissolved in DCM (480 ml) and triethylamine (4.15 g, 41.14 mmol, 5.7 ml) was added. The reaction mixture was stirred at RT for 10 min and 2-(benzyloxy)benzoic acid 3 (9.045 g, 39.67 mmol) was added. The solution was cooled to 0° C. and HOBt (0.54 g, 4 mmol) and DIC (6.26 g, 49.58 mmol, 7.76 ml) were added. The reaction mixture was stirred overnight at RT and a precipitate (diisopropyl urea) was observed. The reaction mixture was diluted with DCM (200 ml), washed with water (200 ml), saturated NaHCO₃ (200 ml), 5% citric acid (200 ml), water (200 ml) and brine (200 ml) and dried over Na2SO₄. The organic solvent was evaporated in vacuum and the crude residue dissolvend in EA (100 ml) and a precipitate of diisopropyl urea was filtered out (3 g). The organic filtrate was evaporated to obtain dark yellow oil (17 g), which was purified by flash chromatography: SiO₂ (400 ml), CHCl₃, 2% MeOH in CHCl₃to obtain 11.27 g of 5 as oil. Yield: 82.87%.

Synthesis of ethanaminium (2S,3R)-2-(2-(benzyloxy)benzamido)-3-hydroxybutanoate (7): (2S,3R)-Methyl 2-(2-(benzyloxy)benzamido)-3-hydroxybutanoate5 (4.03 g, 11.75 mmol) was dissolved in MeOH (100 ml) and a solution of NaOH (1.88 g, 47 mmol) in water (18 ml) was gradually added with stirring. The reaction mixture was stirred at RT for 2 h and the solvent was evaporated. The oily residue was dissolved in waster (15 ml) and 1N HCl (50 ml) solution was added to precipitate the organic acid 6 ((2S,3R)-2-(2-(benzyloxy)benzamido)-3-hydroxybutanoic acid). The precipitate was filtered and washed with water. 6 was dissolved in EA (500 ml), dried over Na₂SO₄and evaporated in vacuum. The solid was dissolved in MeOH (100 ml) and 70% H₂NEt (2 ml) were added. The solution was evaporated and the residue was dissolved again in MeOH (50 ml) and evaporated. The crude residue was dissolved in MeOH (100 ml) and toluene (10 ml) and evaporated in vacuum to obtain the ethanammonium salt 7 (3.9 g). Yield: 88.8%.

Synthesis of 2-(benzyloxy)-N-((2S,3R)-1-(ethylamino)-3-hydroxy-1-oxobutan-2-yl)benzamide (8): Ethanaminium (2S,3R)-2-(2-(benzyloxy)benzamido)-3-hydroxybutanoate7 (N-Ethylammonium salt) (3.9 g, 10.43 mmol) was dissolved in DCM (200 ml) and the solution was cooled in an ice bath. HOBt (0.135 g, 1.0 mmol) and DCC (2.68 g, 13.03 mmol) were added at 0° C. The reaction mixture was stirred overnight at RT. The precipitate of DCU was filtered (2.02 g). The filtrate was evaporated in vacuum to obtain solid residue (4.6 g). The residue was dissolved in CHCl₃ (100 ml), washed with water (50 ml) and dried over Na₂SO₄. The crude product was purified by column chromatography: SiO₂ (70 ml), 2% MeOH in CHCl₃ to obtain 3.68 g of 8. Yield: 99.2%.

Synthesis of (4S,5S)-2-(2-(benzyloxy)phenyl)-N-ethyl-5-methyl-4,5-dihydrooxazole-4-carboxamide (9):2-(Benzyloxy)-N-((2S,3R)-1-(ethylamino)-3-hydroxy-1-oxobutan-2-yl)benzamide 8 (3.68 g, 10.33 mmol) was dissolved DCM (70 ml) in 0.5 L flask and thionyl chloride (24.6 g, 207 mmol, 15 ml) was added with stirring and cooling in an ice bath. The reaction mixture was stirred overnight at RT. The reaction mixture was diluted with EA (30 ml) and evaporated in vacuum. The residue was dissolved in EA (20 ml) and the solution was evaporated. The residue was dissolved in CHCl₃ (100 ml) and dry Na₂CO₃ (8 g) was added and the reaction was stirred for 1 h. Dry Na₂CO₃ (8 g) was added again and the mixture stirred for 1 h. 5 more portions of Dry Na₂CO₃were added and the mixture stirred for 3 days. The precipitate was filtered and washed with CHCl₃ and the solvent evaporated in vacuum to obtain light brown solid product (3.45 g) which was purified by column chromatography: SiO₂ (70 ml), Hexane:EA 1:2 to obtain 2.36 g of 9. Yield: 67.6%.

Synthesis of (4S,5S)-N-ethyl-2-(2-hydroxyphenyl)-5-methyl-4,5-dihydrooxazole-4-carboxamide (10): (4S,5S)-2-(2-(benzyloxy)phenyl)-N-ethyl-5-methyl-4,5-dihydrooxazole-4-carboxamide 9 (2.38 g, 6.98 mmol) was suspended in EtOH (100 ml) and 10% NYC (0.7 g) were added. The reaction mixture was stirred under hydrogen atmosphere at 1 atm for 3 h. The reaction mixture was filtered and the filtrate was evaporated to obtain 10 as solid (1.64 g). Yield: 94.8%. 1H NMR (250 MHz, CDCl₃□ ppm 11.56 (s, 1H), 7.69 (d, J=7.86 Hz, 1H), 7.43 (t, J=7.81 Hz, 1H), 7.03 (d, J=8.37 Hz, 1H), 6.92 (t, J=7.61 Hz, 1H), 6.47 (s, 1H), 5.46-5.02 (m, 1H), 4.93 (d, J=10.27 Hz, 1H), 3.74-2.95 (m, 1H), 1.39 (d, J=6.46 Hz, 1H), 1.16 (t, J=7.77 Hz, 1H).

### Example 2

### Synthesis of D-cis (4R, 5R) POxA Ligand

### D-cis (4R, 5R)

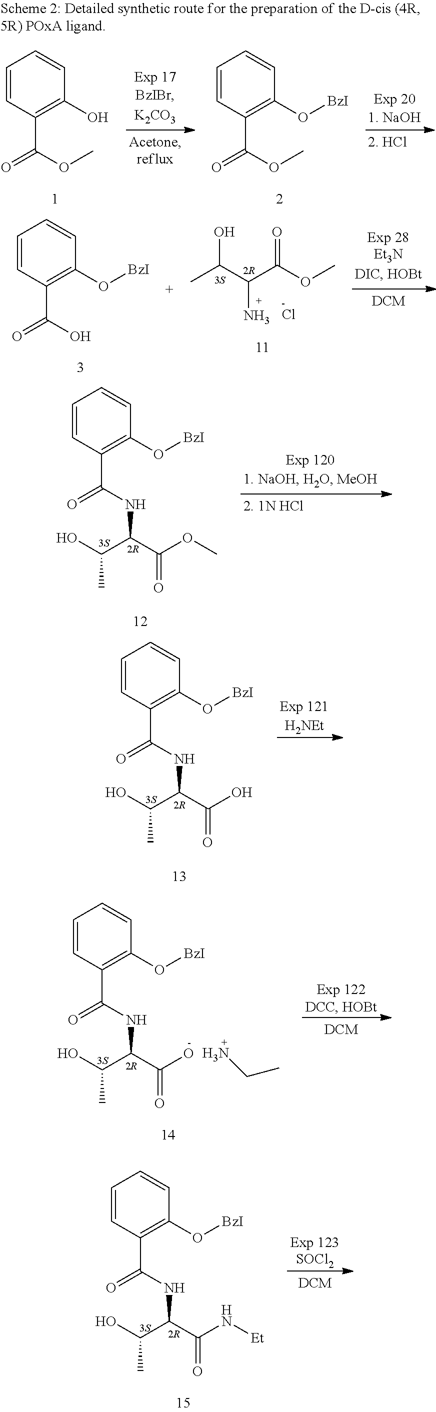

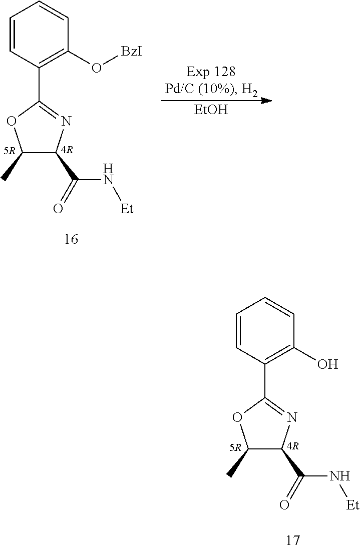

Synthesis of (2R,3S)-methyl 2-(2-(benzyloxy)benzamido)-3-hydroxybutanoate (12): D-Threonine methyl ester•HCl ((2R,38)-methyl 2-amino-3-hydroxybutanoate), 11, (13.94 g, 82.24 mmol) was dissolved in DCM (960 ml) and triethylamine (8.3 g, 82.24 mmol, 11.4 ml) was added. The reaction mixture was stirred at RT for 10 min and 2-(benzyloxy)benzoic acid 3(18.09 g, 79.34 mmol) was added. The solution was cooled to 0° C. and HOBt (1.08 g, 8 mmol) and DIC (12.51 g, 99.16 mmol, 15.5 ml) were added. The reaction mixture was stirred overnight at RT and a precipitate (diisopropyl urea) was observed. The reaction mixture was diluted with DCM (400 ml), washed with water (400 ml), saturated NaHCO₃ (400 ml), 5% citric acid (400 ml), water (400 ml) and brine (400 ml) and dried over Na₂SO₄. The organic solvent was evaporated in vacuum and the residue dissolvend in EA (200 ml) and a precipitate of diisopropyl urea was filtered out (5 g). The organic filtrate was evaporated to obtain dark yellow oil (35 g). The crude product was purified by flash chromatography: SiO₂ (600 ml), CHCl₃, 2% MeOH in CHCl₃ to obtain 23.6 g of 12 as oil. Yield: 92.9%.

Synthesis of (2R,3S)-2-(2-(benzyloxy)benzamido)-3-hydroxybutanoic acid (13): (2R,3S)-Methyl 2-(2-(benzyloxy)benzamido)-3-hydroxybutanoate 12 (5.1 g, 14.87 mmol) was dissolved in MeOH (200 ml) and a solution of NaOH (2.38 g, 60 mmol) in water (30 ml) was gradually added with stirring. The reaction mixture was stirred at RT for 2 h and the solvent was evaporated. The oily residue was dissolved in waster (10 ml) and 2.5N HCl solution was added to pH=1 to precipitate the organic acid 13. The precipitate was filtered, washed with water and dissolved in EA (600 ml). The organic layer was washed with water (50 ml), dried over Na₂SO₄ and evaporated in vacuum. The solid residue was dried in high vacuum to obtain 13 as solid (4.5 g). Yield: 92%.

Ethanaminium (2R,3S)-2-(2-(benzyloxy)benzamido)-3-hydroxybutanoate (14): (2R,3S)-2-(2-(benzyloxy)benzamido)-3-hydroxybutanoic acid 13 (4.5 g) was dissolved in MeOH (100 ml) and 70% H₂NEt (2 ml) were added. The solution was evaporated and the residue was dissolved again in MeOH (50 ml) and evaporated. The residue was dissolved in MeOH (100 ml) and toluene (10 ml) and evaporated in vacuum to obtain the ethanammonium salt 14(5.2 g).

2-(benzyloxy)-N-((2R,3S)-1-(ethylamino)-3-hydroxy-1-oxobutan-2-yl)benzamide (15): Ethanaminium (2R,3S)-2-(2-(benzyloxy)benzamido)-3-hydroxybutanoate (N-Ethylammonium salt) 14 (5.2 g, 13.9 mmol) was dissolved in DCM (150 ml) and the solution was cooled in an ice bath. HOBt (0.187 g, 1.39 mmol) and DCC (3.58 g, 17.4 mmol) were added at 0° C. The reaction mixture was stirred overnight at RT. The precipitate of DCU was filtered (2.02 g). The filtrate was diluted with DCM (70 ml) and washed with water (40 ml). The organic layer was dried over Na₂SO₄ and the solvent was evaporated in vacuum to obtain the ethyl amidel5 as solid (6.1 g).

(4R,5R)-2-(2-(benzyloxy)phenyl)-N-ethyl-5-methyl-4,5-dihydrooxazole-4-carboxamide (16): 2-(Benzyloxy)-N-((2R,3S)-1-(ethylamino)-3-hydroxy-1-oxobutan-2-yl)benzamide 15 (6.1 g, 14 mmol) was dissolved DCM (100 ml) in 0.5 L flask and thionyl chloride (33.3 g, 280 mmol, 20.4 ml) was added with stirring and cooling in an ice bath. The reaction mixture was stirred overnight at RT. The reaction mixture was diluted with CHCl₃ (30 ml) and evaporated in vacuum. The residue was dissolved in EA (40 ml) and the solution was evaporated. The residue was dissolved in CHCl₃ (80 ml) and dry Na₂CO₃ (10 g) was added and the reaction was stirred for 1 h. Dry Na₂CO₃ (10 g) was added again and the mixture stirred for 1 h. one more portions of Dry Na₂CO₃were added and the mixture stirred overnight. The precipitate was filtered and washed with CHCl₃ and the solvent evaporated in vacuum to obtain light brown solid product (˜8 g) which was purified by column chromatography: SiO₂ (100 ml), Hexane:EA 1:1 to obtain 2.3 g of 16 as solid. Yield: 48.9%.

(4R,5R)-N-ethyl-2-(2-hydroxyphenyl)-5-methyl-4,5-dihydrooxazole-4-carboxamide (17): (4R,5R)-2-(2-(benzyloxy)phenyl)-N-ethyl-5-methyl-4,5-dihydrooxazole-4-carboxamidel6 (2.3 g, 6.79 mmol) was dissolved in EtOH (140 ml) and 10% Pd/C (0.69 g) were added. The reaction mixture was stirred under hydrogen atmosphere at 1 atm for 3 h. The reaction mixture was filtered and the filtrate was evaporated. The residue was dissolved in CHCl₃ (20 ml) and toluene (10 ml) and the solution was evaporated in vacuum and dried in high vacuum to obtain 17 as solid (1.6 g). Yield: 95.2%. 1H NMR (250 MHz, CDCl₃) δ ppm 11.58 (s, 1H), 7.70 (d, J=7.87 Hz, 1H), 7.44 (t, J=7.87 Hz, 1H), 7.04 (d, J=8.5 Hz, 1H), 6.92 (t, J=7.5 Hz, 1H), 6.48 (s, 1H), 5.22-5.13 (m, 1H), 4.94 (d, J=10.25 Hz, 1H), 3.43-3.28 (m, 1H), 1.40 (d, J=6.5 Hz, 1H), 1.17 (t, J=7.25 Hz, 1H).

### Example 3

### Synthesis of L-trans (4R, 5S) POxA Ligand

### L-trans (4R, 5S)

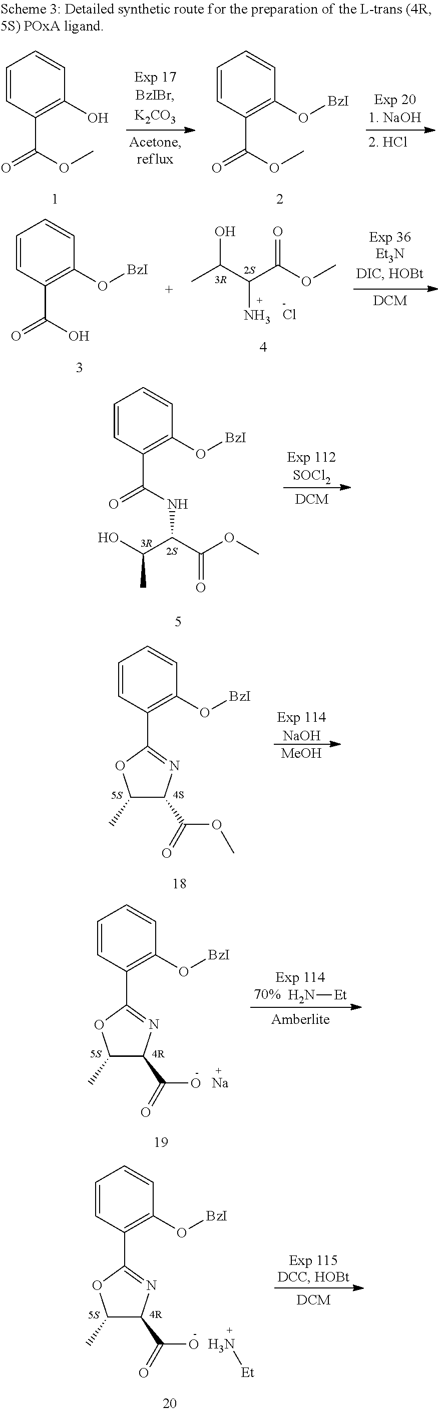

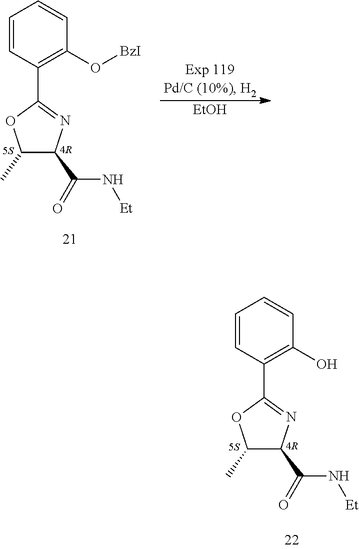

Synthesis of (4S,5S)-methyl 2-(2-(benzyloxy)phenyl)-5-methyl-4,5-dihydrooxazole-4-carboxylate (18): (2S,3R)-methyl 2-(2-(benzyloxy)benzamido)-3-hydroxybutanoate 5(4 g, 11.6 mmol) was dissolved DCM (75 ml) in 0.5 L flask and thionyl chloride (13.8 g, 116 mmol, 8.47 ml) was added with stirring and cooling in an ice bath. The reaction mixture was stirred overnight at RT. The reaction mixture was diluted with EA (20 ml) and evaporated in vacuum. The residue was dissolved in EA (20 ml) and the solution was evaporated. The residue was dissolved in CHCl₃ (70 ml) and dry Na₂CO₃ (7 g) was added and the reaction was stirred for 1 h. Dry Na₂CO₃ (7 g) was added again and the mixture stirred for 2 days. The precipitate was filtered and the solvent evaporated in vacuum to obtain 18 as oil (4.24 g). Yield: 112.4%.

Synthesis of ethanaminium (4R,5S)-2-(2-(benzyloxy)phenyl)-5-methyl-4,5-dihydrooxazole-4-carboxylate (20): (4S,5S)-Methyl 2-(2-(benzyloxy)phenyl)-5-methyl-4,5-dihydrooxazole-4-carboxylate 18 (4.2 g, 11.6 mmol) was dissolved in MeOH (10 ml) and 2N NaOH (25 ml, 50 mmol) were added with stirring and cooling in an ice bath. The reaction mixture was stirred for 2 h at RT and the solvents were evaporated in vacuum. The residue 19 was dissolved in water (20 ml) and placed on top of Amberlite IR-120 column H₃N⁺Et form. The resin column was eluted with water (7×25 ml). After evaporation, MeOH and toluene (200 ml) were added to the residue and the solvents were evaporated to obtain the solid ethanaminium salt 20 (4.2 g). Yield: 101.7%.

Synthesis of (4R,5S)-2-(2-(benzyloxy)phenyl)-N-ethyl-5-methyl-4,5-dihydrooxazole-4-carboxamide (21): Ethanaminium (4R,5S)-2-(2-(benzyloxy)phenyl)-5-methyl-4,5-dihydrooxazole-4-carboxylate (N-Ethylammonium salt) 20 (4.2 g, 11.6 mmol) was dissolved in DCM (100 ml) and the solution was cooled in an ice bath. HOBt (0.157 g, 1.16 mmol) and DCC (2.99 g, 14.5 mmol) were added at 0° C. The reaction mixture was stirred overnight at RT. The precipitate of DCU was filtered (2.5 g). The filtrate was diluted with DCM (100 ml), washed with water (40 ml), dried over Na₂SO₄ and evaporated. The residue (6 g) was treated with EA (60 ml) and precipitate of DCU was filtered. The filtrate was evaporated in vacuum and the residue of crude product (4.87 g) was purified by column chromatography: SiO₂ (100 ml), CHCl₃ to obtain 1.94 g of the ethanamide 21. Yield: 49.4%.

Synthesis of (4R,5S)-N-ethyl-2-(2-hydroxyphenyl)-5-methyl-4,5-dihydrooxazole-4-carboxamide (22): (4R,5S)-2-(2-(benzyloxy)phenyl)-N-ethyl-5-methyl-4,5-dihydrooxazole-4-carboxamide 21 (1.94 g, 5.73 mmol) was dissolved in EtOH (100 ml) and 10% Pd/C (0.58 g) were added. The reaction mixture was stirred under hydrogen atmosphere at 1 atm for 2 h. The reaction mixture was filtered and the filtrate was evaporated to obtain 22 as solid (1.3 g). Yield: 91.5%. 1H NMR (400 MHz, CDCl₃) δ ppm 11.60 (s, 1H), 7.71 (d, 1H), 7.47 (t, 1H), 7.05 (d, 1H), 6.93 (t, 1H), 6.37 (s, 1H), 4.92-4.87 (m, 1H), 4.40 (d, 1H), 3.41-3.36 (m, 1H), 1.63 (d, 1H), 1.18 (t, 1H).

### Example 4

### Synthesis of D-trans (4S, 5R) POxA Ligand

### D-trans (4S, 5R) Synthesis of (4S,5R)-N-ethyl-2-(2-hydroxyphenyl)-5-methyl-4,5-dihydrooxazole-4-carboxamide

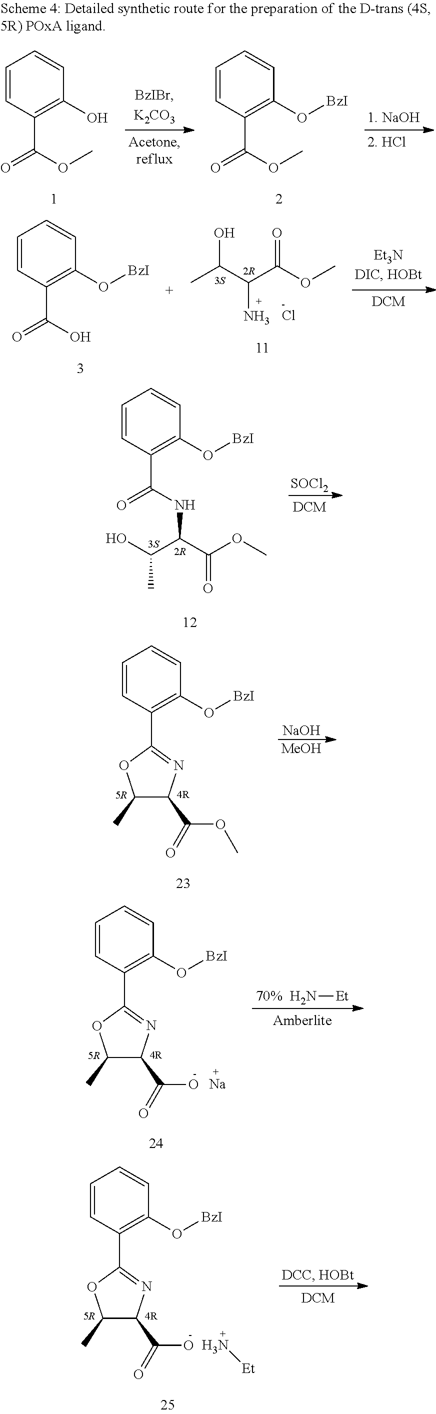

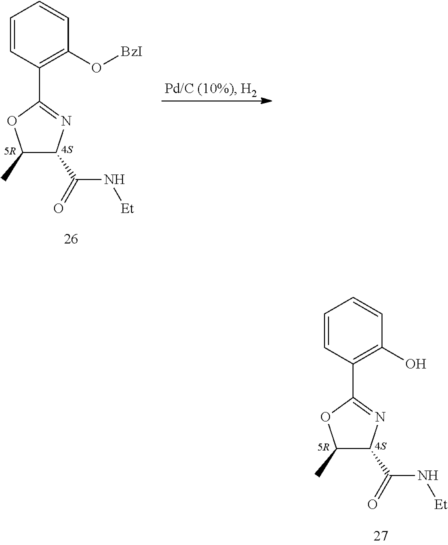

Synthesis of Sodium (4S,5R)-2-(2-(benzyloxy)phenyl)-5-methyl-4,5-dihydrooxazole-4-carboxylate (23): Methyl 2-(2-(benzyloxy)benzamido)-3-hydroxybutanoate 12 (5 g, 14.55 mmol) was dissolved DCM (100 ml) in 0.5 L flask and thionyl chloride (33.3 g, 280 mmol, 20.4 ml) was added with stirring and cooling in an ice bath. The reaction mixture was stirred overnight at RT. The reaction mixture was diluted with CHCl₃ (20 ml) and evaporated in vacuum. The residue was dissolved in EA (20 ml) and the solution was evaporated (repeated twice). The residue was dissolved in CHCl₃ (100 ml) and dry Na₂CO₃ (10 g) was added and the reaction was stirred for 1 h. Dry Na₂CO₃ (7 g) was added again and the mixture stirred overnight. The precipitate was filtered and the solvent evaporated in vacuum to obtain oily product which was purified by column chromatography: SiO₂ (100 ml), Hexane:EA 1:1 to obtain 2.3 g of 23. Yield: 48.9%.

Synthesis of ethanaminium (4R,5R)-2-(2-(benzyloxy)phenyl)-5-methyl-4,5-dihydrooxazole-4-carlboxylate (25): (4R,5R)-Methyl 2-(2-(benzyloxy)phenyl)-5-methyl-4,5-dihydrooxazole-4-carboxylate 23 (5 g, 14.55 mmol) was dissolved in MeOH (150 ml) and 2N NaOH (30 ml, 58.2 mmol) were added with stirring and cooling in an ice bath. The reaction mixture was stirred for 2 h at RT to form sodium (4R,5R)-2-(2-(benzyloxy)phenyl)-5-methyl-4,5-dihydrooxazole-4-carboxylate 24. The MeOH was evaporated in vacuum. The aqueous solution of the sodium salt 24 was placed on top of Amberlite IR-120 column in H₃N⁺Et form. The resin column was eluted with water. MeOH (200 ml) was added to the ammonium salt fractions collected, from the column and was evaporated. The, residue was dissolved in MeOH (100 ml) and evaporated in vacuum 4 more times. The residue was dissolved in CHCl₃ (30 ml) and toluene (10 ml) and the solution was evaporated in vacuum and in high vacuum to obtain 25 (5 g). Yield: 95.52%.

Synthesis of (4S,5R)-2-(2-(benzyloxy)phenyl)-N-ethyl-5-methyl-4,5-dihydrooxazole-4-carboxamide (26): Ethanaminium (4S,5R)-2-(2-(benzyloxy)phenyl)-5-methyl-4,5-dihydrooxazole-4-carboxylate(N-Ethylammonium salt) 25 (5 g, 14.55 mmol) was dissolved in DCM (100 ml) and the solution was cooled in an ice bath. HOBt (0.195 g, 1.45 mmol) and DCC (3.74 g, 18.18 mmol) were added at 0° C. The reaction mixture was stirred overnight at RT. The precipitate of DCU was filtered (3.4 g). The filtrate was diluted with DCM (100 ml), washed with water (40 ml), dried over Na₂SO₄ and evaporated. The residue (7.17 g) was treated with EA (150 ml) and precipitate of DCU was filtered (0.5 g). The filtrate was evaporated in vacuum and the residue of crude product was purified by column chromatography: SiO₂ (120 ml), Hexane:EA 2:1, 1:1 to obtain 2.3 g of 26. Yield: 56.9%.

Synthesis of (4S,5R)-N-ethyl-2-(2-hydroxyphenyl)-5-methyl-4,5-dihydrooxazole-4-carboxamide (27): (4S,5R)-2-(2-(benzyloxy)phenyl)-N-ethyl-5-methyl-4,5-dihydrooxazole-4-carboxamide 26 (2.8 g, 8.27 mmol) was dissolved in EtOH (150 ml) and 10% Pd/C (0.8 g) were added. The reaction mixture was stirred under hydrogen atmosphere at 1 atm for 3 h. The reaction mixture was filtered and the filtrate was evaporated to obtain 27 (1.7 g). Yield: 82.9%. 1H NMR (250 MHz, CDCl₃) δ ppm 7.69 (d, 1H), 7.42 (t, 1H), 7.04 (d, 1H), 6.92 (t, 1H), 6.47 (s, 1H), 4.95-4.90 (m, 1H), 4.40 (d, 1H), 3.37-3.25 (m, 1H), 1.60 (d, 1H), 1.16 (t, 1H).

### Example 5

### Synthesis of 4-nitro and 4-amino L-cis (4S, 5S) POxA Ligand

### Nitro and Amino L-cis (4S, SS) Synthesis of (4S,5S)-N-ethyl-2-(2-hydroxy-4-nitrophenyl)-5-methyl-4,5-dihydrooxazole-4-carboxamide

### and (4S,5S)-2-(4-amino-2-hydroxyphenyl)-N-ethyl-5-methyl-4,5-dihydrooxazole-4-carboxamide

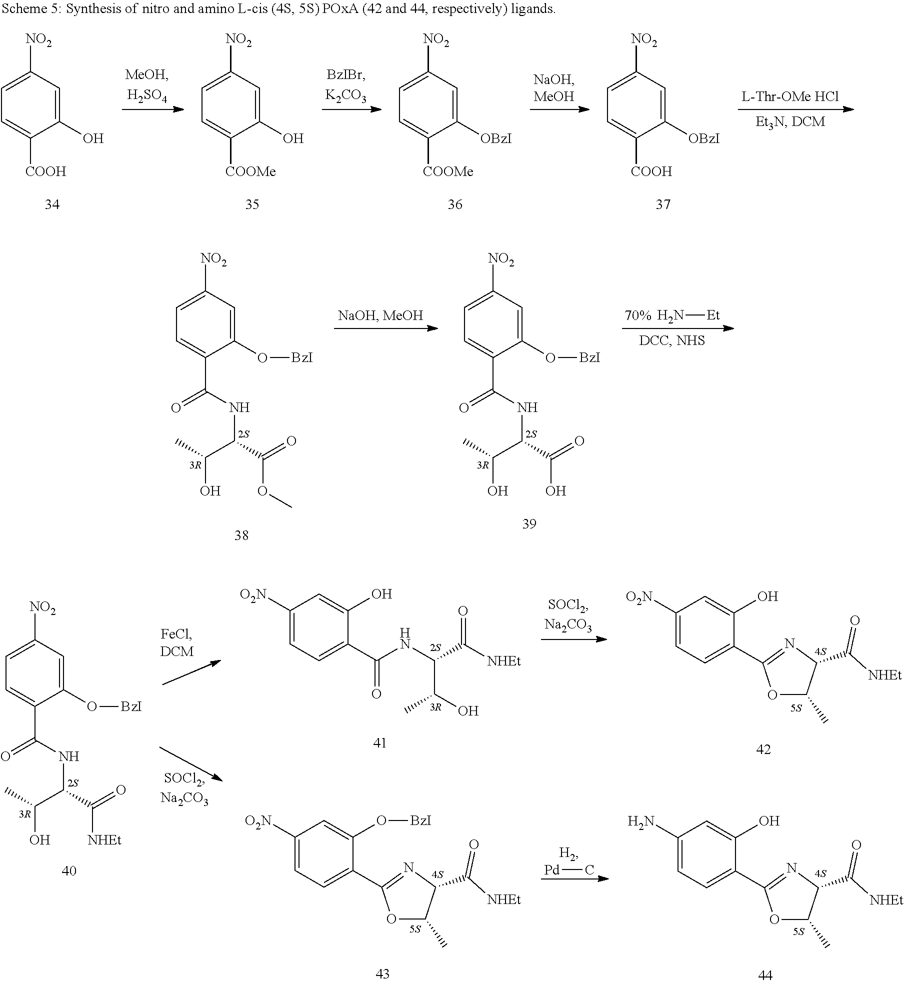

Synthesis of Methyl 2-hydroxy-4-nitrobenzoate (35): Concentrated (98%) sulfuric acid was added to a solution of 2-Hydroxy-4-nitrobenzoic acid 34 (5 g, 27.30 mmol) in MeOH (30 ml) up to pH=1. The reaction mixture was refluxed for 12 hours. MeOH was evaporatedin vacuo, water (5 ml) was added and the mixture was extracted with CHCl₃ (3×50 ml). The combined organic layers were dried over Na₂SO₄, filtered and concentrated in vacuo to obtain the product. Yield: 98%.

Synthesis of Methyl 2-(benzyloxy)-4-nitrobenzoate (36): Anhydrous potassium carbonate (7.41 g, 53.6 mmol) was added to acetone (50 ml) solution containing ester 35 (5.28 g, 26.8 mmol) and benzyl bromide (5.05 g, 29.5 mmol). The reaction mixture was refluxed for 12 hours. Potassium carbonate was filtered off and the solvent was evaporated. The residue was dissolved in EA (50 ml), washed with 1N NaOH, water, brine and dried over Na₂SO₄. Evaporation of solvent afforded 6.67 gr of the title compound. Yield: 87%.

Synthesis of 2-(Benzyloxy)-4-nitrobenzoic acid (37): Methyl 2-(benzyloxy)-4-nitrobenzoate 36 (6.66 g, 23.2 mmol) was dissolved in a mixture of methanol:THF (4:1, 50 ml). 5Naqueous NaOH (46 ml, 230 mmol) was added and the reaction was stirred for 4 hours. The solution was acidified with 1N HCl and the solvents were evaporated. The residue was dissolved in EA (50 ml), washed with water, brine and dried over Na₂SO₄. Evaporation of the solvent afforded 5.90 gr of the product. Yield: 93%.

Synthesis of (2S,3R)-methyl 2-(2-(benzyloxy)-4-nitrobenzamido)-3-hydroxybutanoate (38): (2S,3R)-2-amino-3-hydroxy-butyric acid methyl ester hydrochloride (L-Thr-OMe.HCl) (7.55 g, 32.3 mmol) was dissolved in DCM (50 ml), triethylamine (3.27 g, 32.3 mmol, 4.5 ml) was added and the solution was stirred for 5 minutes. Acid 37 (5.9 g, 21.6 mmol) was added and the mixture was cooled to 0° C. in an ice bath. DCC (5.34 g, 25.9 mmol) and HOBt (878 mg. 6.5 mmol) were added and the reaction mixture was stirred overnight at room temperature. Solvents were evaporated,the residue was dissolved in EA (50 ml), DCU was filtered out, and the supernatant was washed with 1N HCl, brine, dried over Na₂SO₄, filtered and concentratedin vacuo. Flash chromatography with gradient eluent from CHCl₃/Hexan (40%) to CHCl₃/Hexan (10%) afforded 7.96 gr of title compound.Yield: 95%.

Synthesis of (2S,3R)-2-(2-(benzyloxy)-4-nitrobenzamido)-3-hydroxybutanoic acid (39): Compound 38 (7.61 g, 20.5 mmol) was dissolved in methanol (50 ml) and 1N NaOH (41 ml) was added. The reaction mixture was stirred for two hours at room temperature. The reaction was monitored by TLC (6% methanol in CHCl₃; R_{f}(38)=0, R_{f}(39)=0.3). The methanol was evaporated and the aqueous solution was acidified with 1N HCl up to pH=1. The aqueous phase was extracted several times with EA. The organic layers were combined, dried over Na₂SO₄, and evaporated to quantitatively yield 7.65 g of the title compound.

Synthesis of 2-(Benzyloxy)-N-((2S,3R)-1-(ethylamino)-3-hydroxyl-1-oxobutan-2-yl)-4-nitrobenzamide (40): Compound 39 (7.65 g, 20.5 mmol) was dissolved in a mixture of MeOH:THF (4:1, 50 ml), N-hydroxysuccinimide (NHS) (3.07 g, 26.7 mmol) and DCC (5.076 g, 24.6 mmol) were added andthe reaction mixture was stirred for 5 hours at room temperature. Commercial 70% ethylamine in water (7.9 g, 123 mmol) was added and the reaction was stirred overnight at room temperature. Solvents were evaporated and the residue was dissolved in EA (50 ml). DCU was filtered out, the supernatant was washed with 1N HCl, brine, dried over Na₂SO₄, filtered and concentratedin vacuo. Flash chromatography with gradient eluent from CHCl₃:Hexane (3:7) to CHCl₃:Hexan (6:4) afforded 7.65 g of the title compound. Yield: 93%.

Synthesis of N-((2S,3R)-1-(ethylamino)-3-hydroxy-1-oxobutan-2-yl)-2-hydroxy-4-nitrobenzamide (41): Compound 40 (1 g, 2.5 mmol) was dissolved in DCM (20 ml), followed by addition of anhydrous FeCl₃ (1.62 g, 10 mmol) and the reaction mixture was stirred for 1 h at room temperature. Flash chromatography with gradient eluent from CHCl₃/Hexan (95:5) to CHCl₃/Methanol (96:4) afforded 723 mg of title compound. Yield: 93%.

Synthesis of (4S,5S)-N-ethyl-2-(2-hydroxy-4-nitrophenyl)-5-methyl-4,5-dihydrooxazole-4-carboxamide (42): Amide 41 (723 mg, 2.3 mmol) was dissolved in DCM (25 ml) and SOCl₂ (12.5 mmol, 0.9 ml) was added. The reaction mixture was stirred overnight at room temperature. Anhydrous sodium carbonate was added until the solution turned basic. The precipitate was filtered out and the filtrate was concentratedin vacuo. Flash chromatography with gradient from a mixture of 30% hexane in CHCl₃ to 20% hexane in CHCl₃ afforded 566 mg of the title compound. Yield: 83%. 1H NMR (250 MHz, CDCl₃) δ ppm 7.94-7.88 (m, 3H), 7.56-7.34 (m, 5H), 6.67 (s, 1H), 5.35-5.13 (m, 1H), 4.90 (d, J=10.3 Hz, 1H), 3.29-3.00 (m, 2H), 1.33 (d, J=6.5 Hz, 3H), 0.98 (t, J=7.2 Hz, 3H).

Synthesis of (4S,5S)-2-(2-(benzyloxy)-4-nitrophenyl)-N-ethyl-5-methyl-4,5-dihydrooxazole-4-carboxamide (43): Compound 40 (2.33 g, 5.8mmol) was dissolved in DCM (25 ml) and SOCl₂ (58 mmol, 4.2 ml) was added. The reaction mixture was stirred overnight at room temperature. Anhydrous sodium carbonate was added until the solution turned basic. The precipitate was filtered out and the organic phase was concentratedin vacuo. Flash chromatography with gradient eluent from mixture of 40% hexane in CHCl₃ to 20% hexane in CHCl₃ afforded 2.05 gr (85%) of title compound.

Synthesis of (4S,5S)-2-(4-amino-2-hydroxyphenyl)-N-ethyl-5-methyl-4,5-dihydrooxazole-4-carboxamide (44): Compound 43 (805 mg, 2.1 mmol) was dissolved in absolute EtOH (20 ml) and 10% Pd on carbon (240 mg) was added. Hydrogenolysis was carried out at room temperature at 1 atm H₂ for 4 hours. The catalyst was filtered off and evaporation of the solvent afforded 476 mg of the title compound final compound (88%). 1H NMR (250 MHz, Acetone-d₆) δ ppm 7.30 (d, J=8.5 Hz, 1H), 6.29 (dd, J=2.2, 8.5 Hz, 1H), 6.16 (d, J=2.2 Hz, 1H), 5.00-5.11 (m, 1H), 4.77 (d, J=10.0 Hz, 1H), 3.15-3.37 (m, 2H), 1.31 (d, J=6.5 Hz, 3H), 1.09 (t, J=7.2 Hz, 3H).

### Example 6

### Synthesis of 4-azido L-cis (4S, 5S) POxA Ligand

### L-cis (4S, 5S) (4S,5S)-2-(4-azido-2-hydroxyphenyl)-N-ethyl-5-methyl-4,5-dihydrooxazole-4-carboxamide

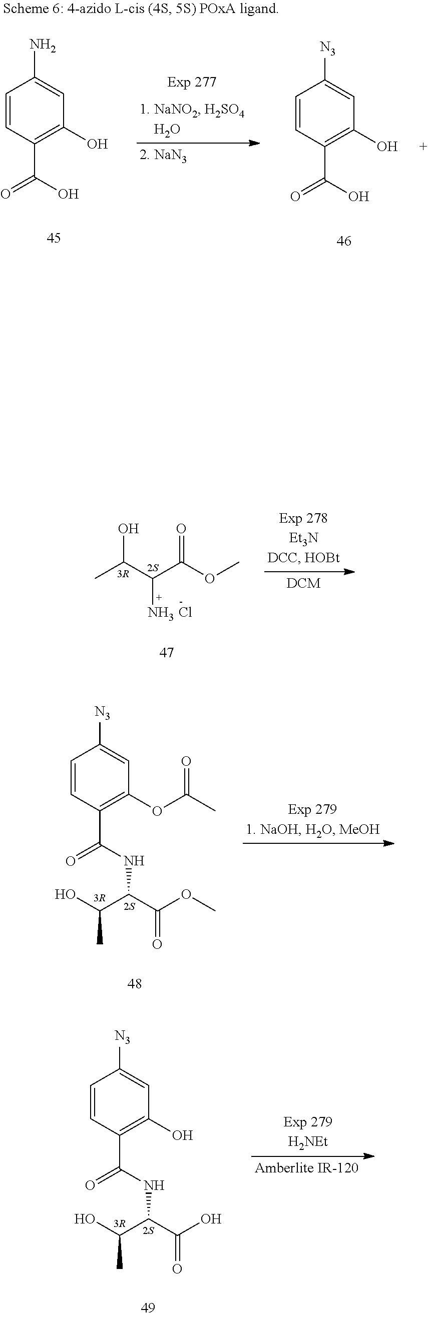

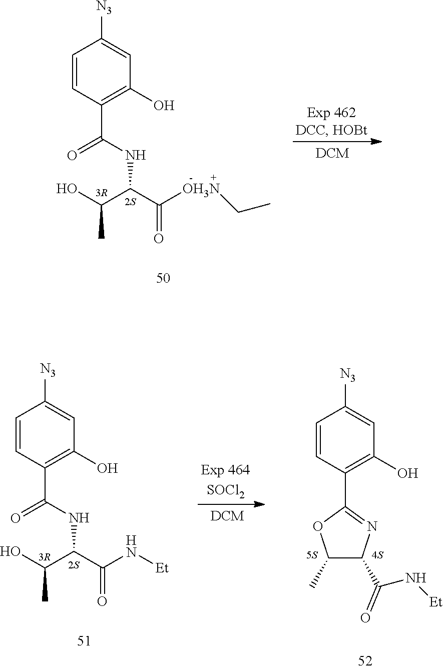

Synthesis of 4-azido-2-hydroxybenzoic acid (46): 4-Amino-2-hydroxybenzoic acid (4-amino-salicylic acid) 45 (5 g, 33 mmol) was added to a solution of H₂SO₄ (25 ml, 46 g, 469 mmol) in water (130 ml). The suspension was stirred and cooled to 0° C. and diazotated by gradual addition of cold solution of NaNO₂ (2.8 g, 40 mmol) in 20 ml water and the reaction mixture was stirred for 1 h at 0° C. A solution of NaN₃ (3.6 g, 56 mmol) in 25 ml water was added to the cooled reaction mixture. Strong and rapid evolution of nitrogen was observed, with formation of precipitate. After the final addition, the reaction mixture was stirred at 0° C. for 1 h. the suspension was allowed to stand overnight at RT. EA (200 ml) was added to the suspension and the organic layer was washed with brine (50 ml) and dried over Na₂SO₄ and evaporated in vacuum to obtain 5.7 g of solid product. Yield: 97.6%.

Synthesis of (2S,3R)-methyl 2-(2-acetoxy-4-azidobenzamido)-3-hydroxybutanoate (48): To a suspension of L-Threonine methyl ester•HCl ((2S,3R)-methyl 2-amino-3-hydroxybutanoate) 47 (439 mg, 2.59 mmol) in DCM (35 ml), triethylamine (261.6 mg, 2.59 mmol, 0.36 ml) was added. The reaction mixture was stirred at RT for 10 min and 4-azido-2-hydroxybenzoic acid 46 (448 mg, 2.5 mmol) was added. The solution was cooled to 0° C. and HOBt (34 mg, 0.25 mmol) and DCC (636 mg, 3.087 mmol) were added. The reaction mixture was stirred overnight at RT and the DCU precipitate was filtered off. The filtrate was diluted with DCM (10 ml) and washed with water (15 ml), 1N NaHCO₃ (15 ml), 5% citric acid (15 ml) and brine (15 ml). The organic layer was dried over Na₂SO₄ and the solvents evaporated in vacuum. The residue (0.9 g) was dissolved in EA (18 ml) and a precipitate of DCU was filtered out (0.06 g). The organic filtrate was evaporated to obtain raw product (0.8 g). The crude product was purified by flash chromatography: SiO₂ (16 g), CHCl₃, 1.5% MeOH in CHCl₃ to obtain 0.56 g of oily product. Yield: 76.2%.

Synthesis of Ethanaminium (2S,3R)-2-(4-azido-2-hydroxybenzamido)-3-hydroxybutanoate (50): (2S,3R)-2-(4-azido-2-hydroxybenzamido)-3-hydroxybutanoic acid (0.55 g, 1.87 mmol) was dissolved in MeOH (10 ml) and 2.5N NaOH (3 ml, 7.48 mmol) were added dropwise with stirring. The reaction mixture was stirred for 2 h at RT and the solvents were evaporated in vacuum. The residue was dissolved in water (10 ml) and was placed on top of 18 g Amberlite IR-120 column in H₃N⁺Et. The resin column was eluted with water. After evaporation of the water, 0.54 g of the title compound was obtained.

Synthesis of 4-Azido-N-((2S,3R)-1-(ethylamino)-3-hydroxy-1-oxobutan-2-yl)-2-hydroxybenzamide (51): To a suspension of L Ethanaminium (2S,3R)-2-(4-azido-2-hydroxybenzamido)-3-hydroxybutanoate (N-Ethylammonium salt 50) (1.28 g, 7.0 mmol) in 25 ml DCM, NEt₃ (0.973 ml, 0.707 g, 7.0 mmol) was added. After 10 min, powder of 4-azidosalicylic acid (1.21 g, 6.756 mmol) was added (did not dissolve). 25 ml of dry DMF was added and the mixture was cooled in an ice bath. HOBt (0.1 g, 0.7 mmol) and DCC (1.8 g, 8.75 mmol) were added to the reaction mixture at 0° C. The reaction mixture was stirred 3 days at rt. Precipitated DCU was filtered. The solvent was evaporated in vacuum and under high vacuum to remove DMF. The residue was treated with 75 ml EA and precipitate of DCU and NEt₃.HCl was filtered (2.5 g). The filtrate was washed with 30 ml water. The dark organic solution was dried over Na₂SO₄ and evaporated. The residue (2.2 g) of crude product was purified by chromatography: SiO₂ (1200 ml), CHCl₃, CHCl₃:MeOH (1.5%) to yield 1.06 g, 51.2% yield.

Synthesis of (4S,5S)-2-(4-azido-2-hydroxyphenyl)-N-ethyl-5-methyl-4,5-dihydrooxazole-4-carboxamide (52): To a stirred solution of 4-azido-N-((2S,3R)-1-(ethylamino)-3-hydroxy-1-oxobutan-2-yl)-2-hydroxybenzamide 51 (1.05 g, 3.42 mmol) in 24 ml DCM, cooled in an ice bath, SOCl₂ (2.48 ml, 4.07 g, 34.2 mmol) was added. The reaction mixture was stirred O.N. at RT. The color of the mixture, changed from yellow to dark brown. The reaction mixture was diluted with CHCl₃ (60 ml) and evaporated in vacuum. The residue was treated with EA (80 ml) and evaporated. The solid residue dissolved in 140 ml CHCl₃ and 4 g dry Na₂CO₃ was added while stirring after 1 h additional 4 g of dry Na₂CO₃ were added. After 4^{th} addition of 4 g dry Na₂CO₃ and stirring for 1 h, the precipitate of Na₂CO₃ was filtered and filtrate was evaporated in vacuum. The orange solid residue was purified by column chromatography: SiO₂ (50 ml), CHCl₃, CHCl₃:MeOH (0.5%). 0.81 g, yield: 81.9%. 1H NMR (300 MHz, CDCl₃) δ ppm 7.66 (d, J=8.48 Hz, 1H), 6.69 (d, J=2.16 Hz, 1H), 6.58 (dd, J=8.46, 2.19 Hz, 1H), 6.45 (s, 1H), 5.23-5.13 (m, 1H), 4.92 (d, J=10.29 Hz, 1H), 3.44-3.27 (m, 1H), 1.39 (d, J=6.52 Hz, 1H), 1.17 (t, J=7.26 Hz, 1H).

### Example 7

### Synthesis of 4-azido D-cis (4R, 5R) POxA Ligand

### D-cis (4R, 5R) (4R,5R)-2-(4-azido-2-hydroxyphenyl)-N-ethyl-5-methyl-4,5-dihydrooxazole-4-carboxamide

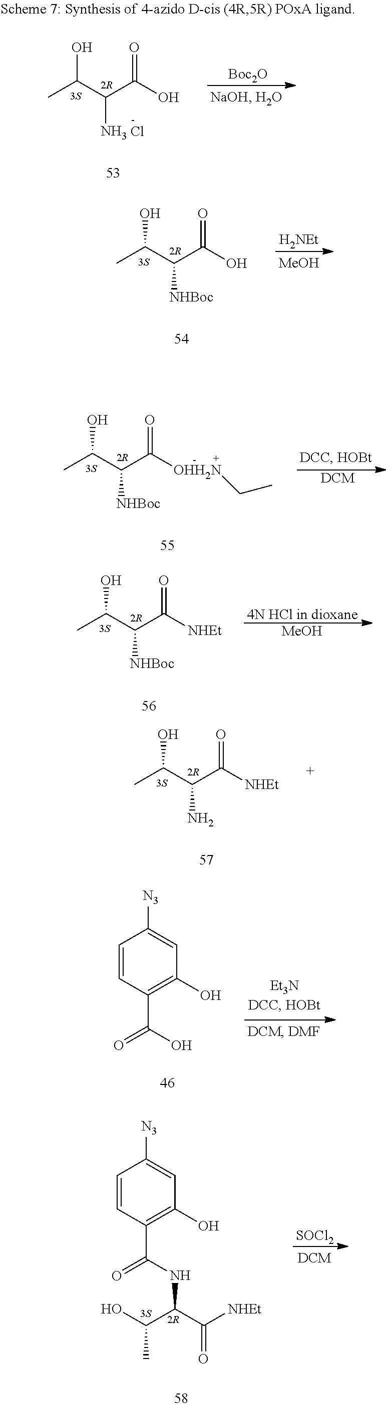

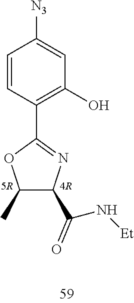 [SMILES: CCNC(=O)[C@@H]1N=C(c2ccc(N=[N+]=[N-])cc2O)O[C@@H]1C]

Synthesis of t-Boc-D-threonine ((2R,3S)-2-(tert-butoxycarbonylamino)-3-hydroxybutanoic acid) (54): A solution of D-Thr-OH((2R,3S)-2-amino-3-hydroxybutanoic acid) 53 (1.78 g, 15 mmol) in a mixture of dioxane (30 ml) and NaOH (1.2 g, (30 mmol) in water (30 ml) was cooled in an ice bath and stirred. (t-Boc)₂O (3.57 g, 16.35 mmol) was added and the reaction mixture was stirred overnight. Reaction mixture was evaporated in vacuum to about 10 ml volume. EA (50 ml) was added to the residue and then while cooling in an ice bath, 1.5N KHSO₄ (20 ml, 30 mmol) was added gradually. The layers were separated and the organic layer was washed with0.5N KHSO₄ (10 ml) and brine (20 ml), dried over Na₂SO₄, the solvent was evaporated in vacuum. The residue was dried under high vacuum. 2.7 g was obtained. Water layer was extracted with EA (20 ml) and the organic solution was washed with brine (10 ml) and dried over Na₂SO₄. 0.49 gwere obtained. Yield: 99.4%.

Synthesis of t-Boc-D-threonine N-ethyl ammonium salt (ethanaminium (2R,3S)-2-(tert-butoxycarbonylamino)-3-hydroxybutanoate) (55): To t-Boc-D-Thr-OH 54 (3.26 g, 17.86 mmol) in MeOH (70 ml), 70% EtNH₂ (3.6 ml, 45 mmol) was added. The solvent was evaporated and the residue dissolved again in MeOH and evaporated. The residue was dissolved in a mixture of MeOH and CHCl₃ and the solution was evaporated. The residue was dissolved in CHCl₃ and evaporated in vacuum. The product is colorless solid (3.67 g).

Synthesis of N-Ethylamide- of N-(test-butoxycarbonyl)-D-threonine (56): Ethyl ammonium salt of t-Boc-D-Thr 55 (3.67 g, 13.88 mmol), was dissolved in 80 ml DCM and the solution was cooled in an ice bath and stirred. HoBt (0.18 g, 1.4 mmol) and DCC (3.54 g, 17.35 mmol) were added at 0° C. and the mixture was stirred for 2 days at rt. The precipitated of DCU (2.9 g) was filtered and the filtrate was evaporated. The residue was treated with 80 ml EA and precipitate of DCU (0.1 gr) was filtered. The solvent was evaporated to obtain 5 g of crude product, which was purified by column chromatography: SiO₂ (200 ml) CHCl₃-MeOH(1%), CHCl₃-MeOH(1.5%) to obtain 2.89 g of the desired product. Yield 83%.

Synthesis of (2R,3S)-2-amino-N-ethyl-3-hydroxybutanamide (57): Solution of tert-butyl (2R,3S)-1-(ethylamino)-3-hydroxy-1-oxobutan-2-ylcarbamate 56 (5.4 g, 21.92 mmol) in MeOH (60 ml) was cooled in an ice bath and 4N HCl in dioxane (22 ml) was added dropwise. The reaction mixture was stirred at RT for 2 h. The reaction solution was evaporated and the residue was dissolved in MeOH (50 ml) and the new solution was evaporated again. The residue was dissolved in CHCl₃(50 ml) and evaporated and dried in high vacuum for 2 h to obtain 4.2 g of solid colorless product.

Synthesis of 4-azido-N-((2R,3S)-1-(ethylamino)-3-hydroxy-1-oxobutan-2-yl)-2-hydroxybenzamide (58): To a suspension of (2R,3S)-2-amino-N-ethyl-3-hydroxybutanamide 57 (2.08 g, 11.38 mmol) in DCM (20 ml), NEt₃ (1.56 ml, 1.15 g, 11.38 mmol) was added. After 10 min, powder of 4-azido-2-hydroxybenzoic acid 46 (1.257 g, 10.92 mmol) was added (did not dissolve). The mixture was evaporated in vacuum and the residue was dissolved in DCM (160 ml) and DMF (35 ml) and cooled in an ice bath. HOBt (0.15 g, 1.1 mmol) and DCC (2.6 g, 13.65 mmol) were added to the reaction mixture at 0° C. The reaction mixture was stirred overnight at RT. Precipitated DCU was filtered. The solvent was evaporated in vacuum and under high vacuum to remove DMF. The residue was treated with EA (150 ml) and precipitate of DCU and NEt₃.HCl was filtered. The filtrate was washed with water (50 ml), dried over Na₂SO₄ and evaporated. The residue (5.3 g) of crude product was purified by chromatography: SiO₂ (200 ml), CHCl₃, CHCl₃:MeOH (1.5%) to yield 2.29 g of the title compound. Yield: 68.5%.

Synthesis of (4R,5R)-2-(4-azido-2-hydroxyphenyl)-N-ethyl-5-methyl-4,5-dihydrooxazole-4-carboxamide (59): To a stirred solution of derivative of 58 (2.25 g, 7.32 mmol) in DCM (50 ml), cooled in an ice bath, SOCl₂ (5.3 ml, 8.71 g, 73.2 mmol) was added. The reaction mixture was stirred overnight at RT. The color of the mixture changed from yellow to dark red. The reaction mixture was diluted with DCM (100 ml) and evaporated in vacuum. The residue was treated with EA (120 ml) and evaporated. The solid residue dissolved in DCM (160 ml) and dry Na₂CO₃(8 g) was added while stirring. After 1 h, additional dry Na₂CO₃(8 g) was added. After 4^{th} addition of dry Na₂CO₃(8 g) and stirring for 1 h, pH of DCM solution >7. Precipitate of Na₂CO₃ was filtered and filtrate was evaporated in vacuum. The red solid residue was purified by column chromatography: SiO₂ (100 ml), CHCl₃, CHCl₃:MeOH (0.5%). Yield: 80.2% (1.7 g). 1H NMR (300 MHz, CDCl₃) δ ppm 7.66 (d, J=8.50 Hz, 1H), 6.68 (d, J=2.12 Hz, 1H), 6.57 (dd, J=8.48, 2.19 Hz, 1H), 6.45 (s, 1H), 5.23-5.13 (m, 1H), 4.93 (d, J=10.14 Hz, 1H), 162-3.06 (m, 1H), 1.40 (d, J=6.50 Hz, 1H), 1.17 (t, J=7.26 Hz, 1H).

### Example 8

### Synthesis of 4-iodo L-cis (4S, 5S) POxA Ligand

### Iodo L-cis (4S, 5S) (4S,5S)-N-ethyl-2-(2-hydroxy-4-iodophenyl)-5-methyl-4,5-dihydrooxazole-4-carboxamide

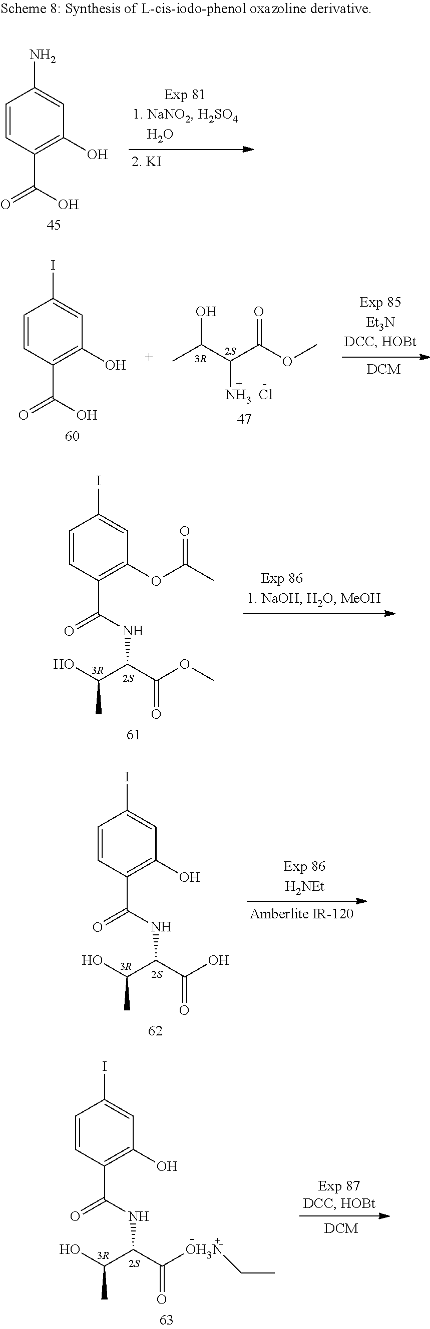

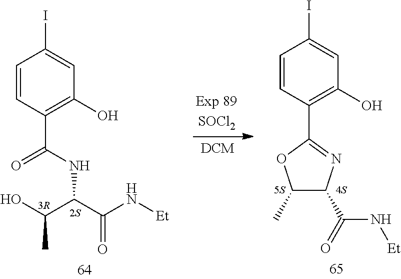 [SMILES: CCNC(=O)[C@@H](NC(=O)c1ccc(I)cc1O)[C@@H](C)O ; CCNC(=O)[C@H]1N=C(c2ccc(I)cc2O)O[C@H]1C]

Synthesis of 4-iodo-salicylic acid (60): In a 0.5 L 3 necked flask, 4-aminosalicylic acid 45 (15.3 g, 100 mmol) was mixed with H₂O (100 ml), Conc. H₂SO₄ (14 ml, 25.8 g, 263 mmol). The mixture was stirred and cooled to 3-5° C. and diazotated by gradual addition of cold solution of NaNO₂ (6.9 g, 100 mmol) in 20 ml water with control with iodine:starch paper of excess NaNO₂. Dark solution was obtained. The diazotated solution was added to cold solution of KI (26 g, 156.6 mmol) in 25 ml 1N H₂SO₄. After 1 min, strong and rapid evolution of nitrogen was observed without heating. Ether (10-20 ml) was added to destroy the foam. The beaker with reaction mixture was heated at 75-80° C. for 10 min. The precipitate was filtered and washed with water and dried in air to obtain 17 g of raw product, which was purified by column chromatography: 340 g SiO₂, 2% MeOH in CHCl₃. 10.5 g, Yield: 39.8%.

Synthesis of (2S,3R)-methyl 2-(2-acetoxy-4-iodobenzamido)-3-hydroxybutanoate (61): To a suspension of L-Threonine methyl ester•HCl ((2S,3R)-methyl 2-amino-3-hydroxybutanoate) (2.45 g, 14.45 mmol) in DCM (195 ml), triethylamine (1.46 g, 14.45 mmol, 2.0 ml) was added. The reaction mixture was stirred at RT for 10 min and 4-iodo salicylic acid (2-hydroxy-4-iodobenzoic acid) (3.68 g, 13.94 mmol) was added. The solution was cooled to 0° C. and HOBt (0.189 g, 1.4 mmol) and DCC (3.58 g, 17.42 mmol) were added. The reaction mixture was stirred overnight at RT and a precipitate of DCU was formed, which was filtered off (3.4 g). The filtrate was diluted with DCM (60 ml), washed with water (90 ml), saturated NaHCO₃ (90 ml), 5% citric acid (90 ml), water (90 ml) and brine (90 ml) and dried over Na₂SO₄. The organic solvent was evaporated in vacuum and the residue treated with EA (100 ml) and a precipitate of DCU was filtered out (0.2 g). The organic filtrate was evaporated to obtain raw material (5 g). The crude product was purified by flash chromatography: SiO₂ (100 ml), CHCl₃:Hexane (1:1), CHCl₃:Hexane (2:1) to obtain 2.6 g of the title compound. Yield: 49.2%.

Synthesis of ethanaminium (2S,3R)-3-hydroxy-2-(2-hydroxy-4-iodobenzamido)butanoate (63): (2S,3R)-methyl 2-(2-acetoxy-4-iodobenzamido)-3-hydroxybutanoate 61 (2.6 g, 6.858 mmol) was dissolved in MeOH (140 ml) and a solution of NaOH (1.37 g, 34.3 mmol) in water (17 ml) was gradually added with stirring. The reaction mixture was stirred at RT for 2 h and the solvent was evaporated. The crude residue of 62 was dissolved in water (30 ml) and the solution was placed on the top of a column prepared from Amberlite IR-120 (NH₃Et) and eluted with water. After evaporation in vacuum, oily product was obtained, which was dissolved in MeOH and CHCl₃. the solution was evaporated and dried in high vacuum to obtain 2.8 of solid product. Yield: 100%.

Synthesis of N-((2S,3R)-1-(ethylamino)-3-hydroxy-1-oxobutan-2-yl)-2-hydroxy-4-iodobenzamide (64): Ethanaminium (2S,3R)-3-hydroxy-2-(2-hydroxy-4-iodobenzamido)butanoate (N-Ethylammonium salt) 63 (2.81 g, 6.858 mmol) was dissolved in DCM (190 ml) and DMF (50 ml) and the solution was cooled in an ice bath. HOBt (92 mg, 0.68 mmol) and DCC (1.76 g, 8.57 mmol) were added at 0° C. The reaction mixture was stirred for 2 days at RT. The solvents were evaporated in high vacuum to remove DMF and the residue was dissolved in CHCl₃ (100 ml) and washed with water (50 ml). The solvent was evaporated and the residue was washed with water (40 ml) to remove DMF. The solid residue was dissolved in EA (100 ml) and dried over Na₂SO₄. The crude product was purified by column chromatography: SiO₂ (60 ml), CHCl₃, 2% MeOH in CHCl₃ to obtain 2.35 g of product. Yield: 87.4%.

Synthesis of (4S,5S)-N-ethyl-2-(2-hydroxy-4-iodophenyl)-5-methyl-4,5-dihydrooxazole-4-carboxamide (65): N-((2S,3R)-1-(ethylamino)-3-hydroxy-1-oxobutan-2-yl)-2-hydroxy-4-iodobenzamide 64 (536 mg, 1.36 mmol) was dissolved DCM (6 ml) and thionyl chloride (3.237 g, 27.2 mmol, 2 ml) was added with stirring and cooling in an ice bath. The yellow reaction mixture was stirred overnight at RT. The reaction mixture was diluted with EA (5 ml) and evaporated in vacuum. The residue was dissolved in EA (5 ml) and CHCl₃ (5 ml) and the solution was evaporated. The residue was dissolved in CHCl₃ (75 ml) and dry Na₂CO₃ (1.5 g) was added and the reaction was stirred for 1 h. Dry Na₂CO₃ (1.3 g) was added again and the mixture stirred for 1 h. 5 more portions of Dry Na₂CO₃were added and the mixture stirred for 1 h. These additions of Dry Na₂CO₃ were repeated until the solution became basic. The precipitate was filtered and the solvent was evaporated in vacuum to obtain light brown solid product (0.7 g) which was purified by column chromatography: SiO₂ (14 g), CHCl₃ to obtain 0.42 g of solid brown product. Yield: 82%. ¹H NMR (300 MHz, CDCl₃) δ ppm 11.55 (s, 1H), 7.40 (d, 1H), 7.34 (d, 1H), 7.23 (dd, 1H), 6.43 (s, 1H), 5.22 (m, 1H), 5.01 (d, 1H), 3.32 (m, 2H), 1.41 (d, 3H), 1.14 (t, 3H).

### Example 9

### Synthesis of 4-iodo L-trans (4R, 5S) POxA Ligand

### Iodo L-trans (4R, 5S) (4R,5S)-N-ethyl-2-(2-hydroxy-4-iodophenyl)-5-methyl-4,5-dihydrooxazole-4-carboxamide

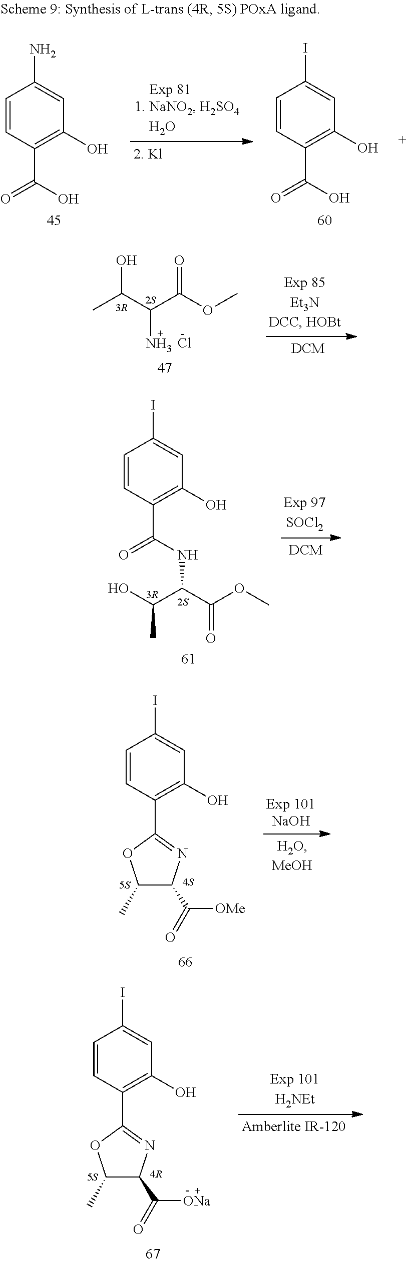

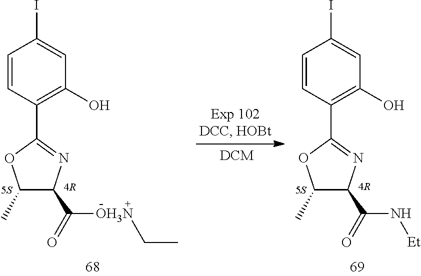

Synthesis of (4S,5S)-methyl 2-(2-hydroxy-4-iodophenyl)-5-methyl-4,5-dihydrooxazole-4-carboxylate (66): To a solution of (2S,3R)-methyl 3-hydroxy-2-(2-hydroxy-4-iodobenzamido)butanoate 65 (3.11 g, 8.2 mmol) in DCM (70 ml), thionyl chloride (19.53 g, 164 mmol, 11.97 ml) was added with stirring and cooling in an ice bath. The reaction mixture was stirred overnight at RT. A new portion of thionyl chloride (6 ml) and the reaction mixture was stirred for another night. The reaction mixture was diluted with EA (30 ml) and evaporated in vacuum. The residue was dissolved in EA (15 ml) and the solution was evaporated. The solid residue was dissolved in CHCl₃ (100 ml) and dry Na₂CO₃ (8 g) was added and the reaction was stirred for 1 h. Dry Na₂CO₃ (8 g) was added again and the mixture stirred until it became basic. The precipitate was filtered and the solvent was evaporated in vacuum to obtain product (2.8 g) which was purified by column chromatography: SiO₂ (60 g), CHCl₃:Hexane (2:1) to obtain 1.96 g of solid brown product 66. Yield: 66.2%.

Synthesis of Ethanaminium (4R,5S)-2-(2-hydroxy-4-iodophenyl)-5-methyl-4,5-dihydrooxazole-4-carboxylate (68): (4S,5S)-methyl 2-(2-hydroxy-4-iodophenyl)-5-methyl-4,5-dihydrooxazole-4-carboxylate 66 (1.58 g, 4.375 mmol) was dissolved in MeOH (140 ml) and a solution of NaOH (0.7 g, 17.5 mmol) in water (9 ml). The reaction mixture was stirred overnight at RT and the solvent was evaporated to obtain the sodium salt 67 (sodium (4R,5S)-2-(2-hydroxy-4-iodophenyl)-5-methyl-4,5-dihydrooxazole-4-carboxylate). 67 was dissolved in water (20 ml) and the solution was placed on the top of a column prepared from Amberlite IR-120 (NH₃Et) and eluted with water. After evaporation in vacuum, oily product was obtained, which was dissolved in MeOH and CHCl₃. the solution was evaporated and dried in high vacuum to obtain 1.89 g of solid product 68. Yield: quantitative.

Synthesis of (4R,5S)-N-ethyl-2-(2-hydroxy-4-iodophenyl)-5-methyl-4,5-dihydrooxazole-4-carboxamide (69): Ethanaminium (4R,5S)-2-(2-hydroxy-4-iodophenyl)-5-methyl-4,5-dihydrooxazole-4-carboxylate (N-Ethylammonium salt) 68 (1.71 g, 4.375 mmol) was dissolved in DCM (100 ml) and the solution was cooled in an ice bath. HOBt (60 mg, 0.437 mmol) and DCC (1.126 g, 5.47 mmol) were added at 0° C. The reaction mixture was stirred overnight at RT. The DCU precipitate was filtered off (0.7 g) and the filtrate was evaporated in vacuum. The residue was dissolved in EA (80 ml) and the precipitate of DCU was filtered off. The filtrate was evaporated to obtain 2.0 g of solid product which was purified by column chromatography: SiO₂ (35 ml), CHCl₃:Hexane (2:1) to obtain 1.3 g of product 69. Yield: 79.4%. 1H NMR (250 MHz, CDCl₃) δ ppm 11.63 (s, 1H), 7.44 (d, J=1.52 Hz, 1H), 7.36 (d, J=8.29 Hz, 1H), 7.25 (dd, J=8.27, 1.53 Hz, 1H), 6.30 (s, 1H), 4.94-4.83 (m, 1H), 4.35 (d, J=7.8Hz, 1H), 3.53-3.12 (m, 1H), 1.60 (d, J=6.27 Hz, 1H), 1.16 (t, J=7.28 Hz, 1H).

### Example 10

### Synthesis of 4-iodo D-cis (4R, 5R) POxA Ligand

### Iodo D-cis (4R, 5R) (4R,5R)-N-ethyl-2-(2-hydroxy-4-iodophenyl)-5-methyl-4,5-dihydrooxazole-4-carboxamide

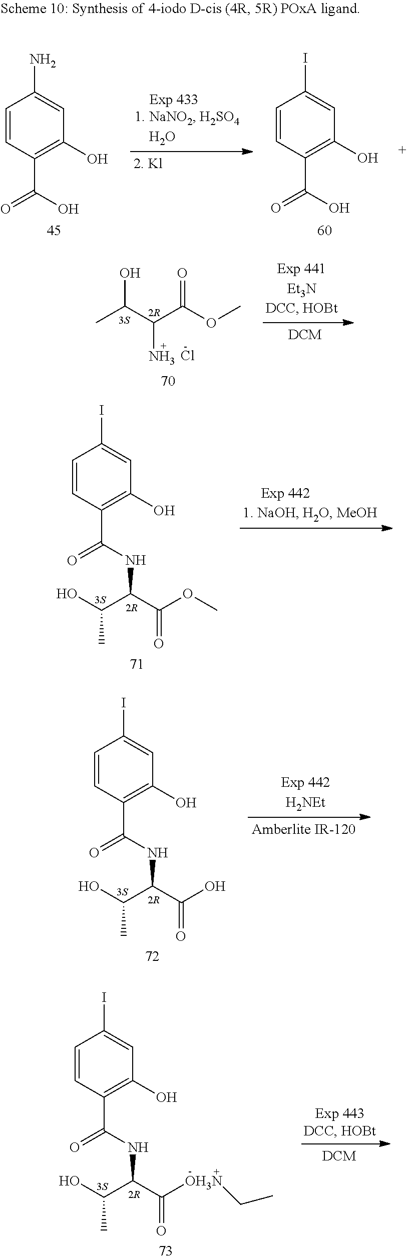

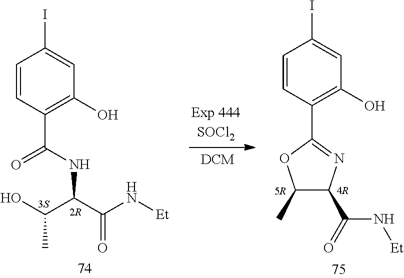 [SMILES: CCNC(=O)[C@H](NC(=O)c1ccc(I)cc1O)[C@H](C)O ; CCNC(=O)[C@@H]1N=C(c2ccc(I)cc2O)O[C@@H]1C]

Synthesis of 4-iodo-salicylic acid (60): 4-aminosalicylic acid 45 (6.12 g, 40 mmol) was mixed with H₂O (40 ml), Conc. H₂SO₄ (5.6 ml, 10 g, 102 mmol). The mixture was stirred and cooled to 3-5° C. and diazotated by gradual addition of cold solution of NaNO₂ (2.76 g, 40 mmol) in 10 ml water with control with iodine:starch paper of excess NaNO₂. Dark solution was obtained. The diazotated solution was added to cold solution of KI (10.3 g, 62.32 mmol) in 10 ml 1N H₂SO₄. After 1 min, strong and rapid evolution of nitrogen was observed without heating. The beaker with reaction mixture was heated at 75-80° C. for 10 min. The precipitate was filtered and washed with water and dried in air to obtain 6.6 g of raw product. Yield: 62.5%.

Synthesis of (2R,3S)-methyl 3-hydroxy-2-(2-hydroxy-4-iodobenzamido)butanoate (71): To a suspension of D-Threonine methyl ester•HCl ((2S,3R)-methyl 2-amino-3-hydroxybutanoate) 70 (1.75 g, 9.61 mmol) in DCM (70 ml), triethylamine (0.97 g, 9.61 mmol, 1.33 ml) was added. The reaction mixture was stirred at RT for 10 min and 4-iodo salicylic acid (2-hydroxy-4-iodobenzoic acid) 60 (2.45 g, 9.28 mmol) was added. The solution was evaporated by vacuum and re-dissolved in DCM (130 ml). the reaction mixture was cooled to 0° C. and HOBt (125 mg, 0.928 mmol) and DCC (2.39 g, 11.6 mmol) were added. The reaction mixture was stirred overnight at RT and a precipitate of DCU was formed, which was filtered off. The filtrate was evaporated in vacuum and treated with EA (100 ml) and precipitate was filtered off. The organic solution was washed with water (20 ml), saturated NaHCO₃ (40 ml), 5% citric acid (40 ml), water (40 ml) and brine (40 ml) and dried over Na₂SO₄. The organic solvent was evaporated in vacuum to obtain 3.8 g of red oil. The crude product was purified by flash chromatography: SiO₂ (80 ml), CHCl₃:Hexane (2:1), CHCl₃, 2% MeOH in CHCl₃, to obtain 2.46 g of the title compound. Yield: 70%.

Synthesis of ethanaminium (2R,3S)-3-hydroxy-2-(2-hydroxy-4-iodobenzamido)butanoate (73): (2R,3S)-methyl 3-hydroxy-2-(2-hydroxy-4-iodobenzamido)butanoate 71 (2.46 g, 6.42 mmol) was dissolved in MeOH (90 ml) and a solution of NaOH (1.37 g, 34.3 mmol) in water (17 ml) was gradually added with stirring. The reaction mixture was stirred at RT for 2 h. the reaction mixture was acidified to pH=1-2 with 5N HCl (8 ml) and the solvent was evaporated. The crude residue was dissolved in water (30 ml), extracted to EA (100 ml) and dried over Na₂CO₃. The residue was treated with CHCl₃ (50 ml) and the suspension was evaporated in vacuum to obtain 2.33 g of 72. Yield: 98.7%.

72 was dissolved in water (20 ml) and the solution was placed on the top of a column prepared from Amberlite IR-120 (NH₃Et) and eluted with water. After evaporation in vacuum, oily product was obtained, which was dissolved in MeOH and CHCl₃. the solution was evaporated and dried in high vacuum to obtain 2.66 of solid product. Yield: 100%.

Synthesis of N-((2R,3S)-1-(ethylamino)-3-hydroxy-1-oxobutan-2-yl)-2-hydroxy-4-iodobenzamide (74): ethanaminium (2R,3S)-3-hydroxy-2-(2-hydroxy-4-iodobenzamido)butanoate (N-Ethylammonium salt) 73 (2.66 g, 6.49 mmol) was dissolved in DCM (140 ml) and the solution was cooled in an ice bath. HOBt (87mg, 0.65 mmol) and DCC (1.67 g, 8.11 mmol) were added at 0° C. The reaction mixture was stirred overnight at RT. TLC showed that the starting material did not react completely. DMF (40 ml) was added and the reaction mixture was cooled in an ice bath followed by the addition of DCC (1 g) and HOBt (40 mg). The reaction mixture was stirred overnight. The solvents were evaporated in high vacuum to remove DMF and the residue was treated with EA (100 ml) and the precipitate of DCU was removed by filtration. The filtrate was washed with water (30 ml) and brine (10 ml) and dried over Na₂SO₄. The crude product (4.7 g) was purified by column chromatography: SiO₂ (80 ml), CHCl₃, 2% MeOH in CHCl₃ to obtain 1.7 g of yellow solid product. Yield: 66.9%.

Synthesis of (4R,5R)-N-ethyl-2-(2-hydroxy-4-iodophenyl)-5-methyl-4,5-dihydrooxazole-4-carboxamide (75): N-((2R,3S)-1-(ethylamino)-3-hydroxy-1-oxobutan-2-yl)-2-hydroxy-4-iodobenzamide 74 (1.7 g, 4.33 mmol) was dissolved DCM (26 ml) and thionyl chloride (10.3 g, 86.7 mmol, 6.45 ml) was added with stirring and cooling in an ice bath. The dark yellow reaction mixture was stirred overnight at RT (after 2 h a suspension was formed). The reaction mixture was diluted with EA (15 ml) and CHCl₃ (8 ml) and evaporated in vacuum. The residue was dissolved in CHCl₃ (300 ml) and dry Na₂CO₃ (33 g) was added and the mixture stirred for 1 h. These additions of Dry Na₂CO₃ were repeated until the solution became basic. The precipitate was filtered and the solvent was evaporated in vacuum to obtain light brown solid product (2 g) which was purified by column chromatography: SiO₂ (40 g), CHCl₃, 1% MeOH in CHCl₃, to obtain 1.45 g of solid brown product 75. Yield: 88.1%. %. ¹H NMR (300 MHz, CDCl₃) δ ppm 11.62 (s, 1H), 7.45 (d, J=1.48 Hz, 1H), 7.36 (d, J=8.29 Hz, 1H), 7.26 (dd, J=8.27, 1.51 Hz, 1H), 6.41 (s, 1H), 5.24-5.14 (m, 1H), 4.94 (d, J=10.33 Hz, 1H), 3.55-3.10 (m, 1H), 1.39 (d, J=6.53 Hz, 1H), 1.16 (t, J=7.27 Hz, 1H).

### Example 11

### Synthesis of 4-ethynyl L-cis (4S, 5S) POxA Ligand

### 4-ethynyl-2-hydroxybenzoic Acid

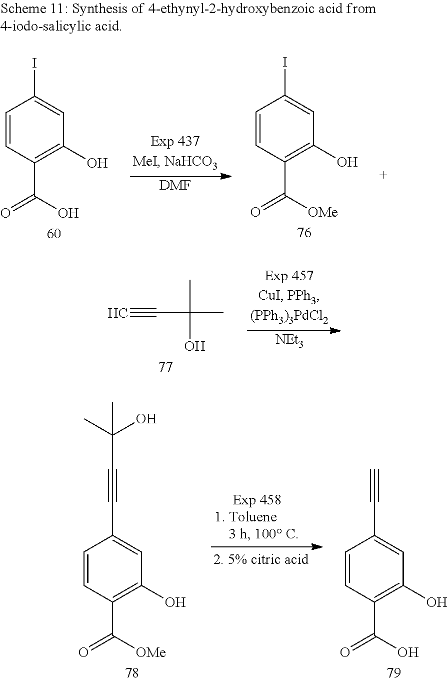

Synthesis of methyl-4-iodosalicylate (76): 4-Iodosalicylic acid 60 (7.3 g, 27.65 mmol) was dissolved in 41 ml DMF and NaHCO₃ (2.78 g, 33.18 mmol) were added (evolution of CO₂) and the mixture stirred for 5 min. MeI (2.66 ml, 5.85 g, 41.47 mmol, 1.5 eq) was added and the reaction mixture was heated to 40° C. for 5 h while stirring (monitored by TLC). Upon reaction completion, the mixture was diluted with 170 ml H₂O and 170 ml EA. The organic layer was subsequently washed with 170 ml of 5% NaHCO₃, 170 ml 5% NaCl and was dried over Na₂SO₄. The solvent was evaporated to give ˜9 g of crude oily product (black color), which was purified by column chromatography: SiO₂ (100 g), Hexane, Hexane:EA 100:2. 5.86 g, Yield: 76.5%.

Synthesis of 4-(3′-hydroxy-4-carboxymethyl)phenyl-3-butyne-2-methy-2-ol (78): A solution of methyl 4-iodosalicylate 76 (5.8 g, 20.8 mmol) and 2-methyl-3-butyn-2-ol 77 (2.44 ml, 2.12 g, 25.2 mmol) in NEt₃ (58 ml) was prepared under N₂. CuI (22 mg), PPh₃ (44 mg) and Pd(PPh₃)Cl₂ (22 mg) were added. The mixture was stirred under reflux for 24 h. NEt₃.HI was precipitated. The reaction mixture was cooled and 450 ml of EA and 170 ml water were added. The organic solution was separated from green water and dried over Na₂SO₄. The yellow organic solution was evaporated to obtain yellow oily residue, which was purified by column chromatography: SiO₂(80 g), Hexane:EA 20:1→2:1 to obtain 3.55 g of solid yellow product. Yield: 73%.

Synthesis of 4-ethynyl salicylic acid or (3-hydroxy-4-carcoxy)phenylacetylene (79): Small grained NaOH (2.1 g, 52.5 mmol) was added to a solution of methyl 2-hydroxy-4-(3-hydroxy-3-methylbut-1-ynyl)benzoate 78 (3.5 g, 14.94 mmol) in toluene (170 ml) with stirring. The reaction mixture was heated to 110° C. for 3 h. After cooling, the reaction suspension was diluted with 300 ml EA and washed with 10% citric acid (40 ml) (pH ˜6-7) and 5% citric acid (20 ml) to pH=2. The organic solution was washed with water (3×60 ml), dried over Na₂SO₄ and evaporated. The residue was treated with DCM (50 ml) and the suspension was evaporated. The solid product was dried under high vacuum to obtain 2.4 g.-yield 99.17%. ¹H NMR (300 MHz, CDCl₃) δ ppm 10.40 (s, 1H), 7.87 (d, J=8.22 Hz, 1H), 7.13 (d, J=1.41 Hz, 1H), 7.03 (dd, J=8.22, 1.48 Hz, 1H), 3.26 (s, 1H).

### Ethynyl L-cis (4S, 5S) (4S,5S)-N-ethyl-2-(4-ethynyl-2-hydroxyphenyl)-5-methyl-4,5-dihydrooxazole-4-carboxamide

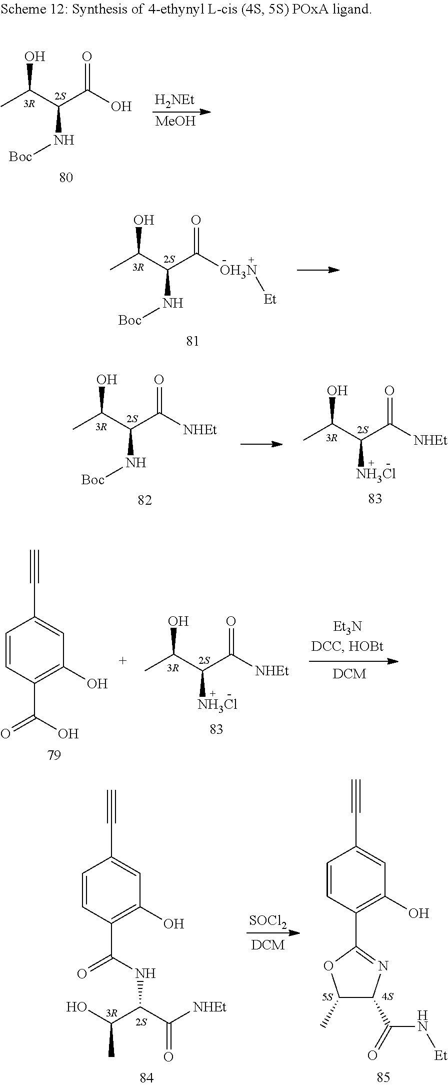

Synthesis of ethanaminium (2S,3R)-2-(tert-butoxycarbonylamino)-3-hydroxybutanoate [(Boc)-L-Thr-N-ethyl ammonium salt] (81): Boc-Thr-OH (5g, 22.8 mmol) was added to a solution of 70% ethylamine (5 ml, 4 gr, 62.1 mmol). The solvent was evaporated and the residue was dissolved in MeOH (100 ml) and the solvent evaporated, then dissolved in a mixture of MeOH and CHCl₃ and again evaporated. After drying in high vacuum, 6.5 g of salt was obtained.

Synthesis of tent-butyl (2S,3R)-1-(ethylamino)-3-hydroxy-1-oxobutan-2-ylcarbamate [(Boc)-L-Thr-NH-Et] (82): The ethylammonium salt 81 (6.5 g, 24.6 mmol) was dissolved in DCM (250 ml) and the solution was cooled to 0° C. with stirring. HOBt (0.332 g, 2.4 mmol) and DCC (6.33 g, 30.73 mmol) were added to the solution and the reaction mixture was stirred at RT overnight. The precipitate of DCU (4.75 g) was filtered off and the filtrate was evaporated. The residue was treated with EA (100 ml), the precipitate of DCU (0.33 g) was filtered and the organic solvent was evaporated. The residue (7.47 g) was purified by column chromatography: SiO₂ 260 ml), 1-3% MeOH in CHCl₃. Some of the fractions were evaporated and of EA (20 ml) was added to further precipitate DCU. Yield: 89.25%, 5.4 g.

Synthesis of N-Ethylamide-L-threonine•HCl (83): Solution of N-(Boc)-L-Thr-NH-Et 82 (5.4 g, 21.92 mmol) in MeOH (60 ml) was cooled in an ice bath and 4N HCl in dioxane (22 ml) was added dropwise. The reaction mixture was stirred at RT for 2 h. The reaction solution was evaporated and the residue was dissolved in MeOH (50 ml) and the new solution was evaporated again. The residue was dissolved in CHCl₃(50 ml) and evaporated and dried in high vacuum for 2 h to obtain 4.2 g of solid colorless product.

Synthesis of N-((2S,3R)-1-(ethylamino)-3-hydroxy-1-oxobutan-2-yl)-4-ethynyl-2-hydroxybenzamide (84): To a suspension of L-threonine N-ethylamide•HCl 83 (2.7 g, 14.8 mmol) in CHCl₃ (260 ml), NEt₃ (2.06 ml, 1.494 g, 14.8 mmol) was added. After 10 min, powder of 4-ethynylsalicylic acid 79 (2.4 g, 14.8 mmol) was added followed by DMF (50 ml). The suspension was cooled in an ice bath. HOBt (0.202 g, 1.5 mmol) and DCC (3.8 g, 18.5 mmol) were added to the reaction mixture at 0° C. The reaction mixture was stirred overnight at RT. The solvent was evaporated in vacuum and under high vacuum to remove DMF. The residue was treated with EA (250 ml) and precipitate of DCU and NEt₃.HCl was filtered (2.5 g). The filtrate was washed with 1N NaHCO₃ (70 ml), water (50 ml), 5% citric acid (50 ml), H₂O (50 ml) and brine (50 ml). The organic solution was dried over Na₂SO₄ and evaporated. The residue (5.8 g) of crude product was purified by chromatography: SiO₂ (100 ml), Hexane:EA 2:1, 1:1, 1:2 to yield 3.0 g, 69.8% yield.

Synthesis of (4S,5S)-N-ethyl-2-(4-ethynyl-2-hydroxyphenyl)-5-methyl-4,5-dihydrooxazole-4-carboxamide (85): To a stirred solution of amide 84 (3 g, 10.33 mmol) in DCM (78 ml), cooled in an ice bath, SOCl₂ (7.5 ml, 12.3 g, 103.3 mmol) was added. The reaction mixture was stirred overnight at RT. The color of the mixture changed from yellow to brown. The reaction mixture was diluted with CHCl₃(100 ml) and evaporated in vacuum. The residue was treated with EA (130 ml) and evaporated. The solid residue dissolved in CHCl₃ (450 ml) and dry Na₂CO₃ (13 g) was added while stirring after 1 h additional dry Na₂CO₃ (13 g) were added. After 3^{rd} addition of dry Na₂CO₃ (13 g) and stirring overnight, the precipitate of Na₂CO₃ was filtered and filtrate was evaporated in vacuum. The orange solid residue was purified by column chromatography: SiO₂ (100 ml), CHCl₃, CHCl₃:MeOH (0.5%). 0.81 g, yield: 75.4%. ¹H NMR (300 MHz, CDCl₃) δ ppm 11.58 (s, 1H), 7.63 (d, J=8.10 Hz, 1H), 7.15 (d, J=1.40 Hz, 1H), 7.02 (dd, J=8.10, 1.48 Hz, 1H), 6.44 (s, 1H), 5.24-5.14 (m, 1H), 4.93 (d, J=10.32 Hz, 1H), 3.66-3.25 (m, 2H), 3.20 (s, 1H), 1.40 (d, J=6.52 Hz, 3H), 1.16 (t, J=7.26 Hz, 3H).

### Example 12

### Synthesis of 4-ethynyl D-cis (4R, 5R) POxA Ligand

### Ethyny D-cis (4R, 5R) (4R,5R)-N-ethyl-2-(4-ethynyl-2-hydroxyphenyl)-5-methyl-4,5-dihydrooxazole-4-carboxamide

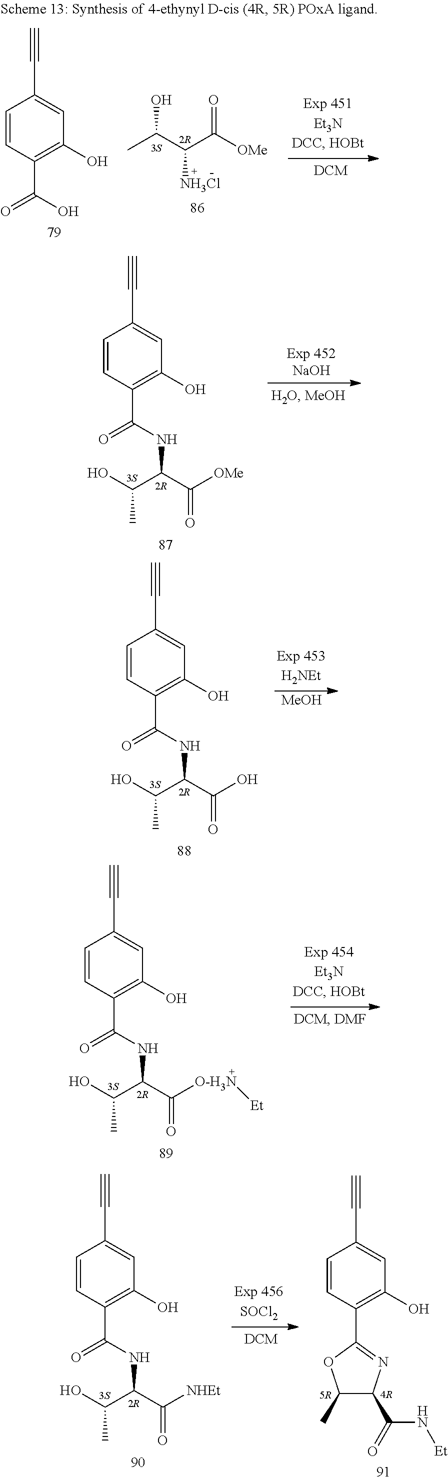

Synthesis of (2R,3S)-methyl 2-(4-ethynyl-2-hydroxybenzamido)-3-hydroxybutanoate (87): To a suspension of L-threonine methyl ester•HCl 86 (2.7 g, 14.8 mmol) in CHCl₃ (260 ml), NEt₃ (2.06 ml, 1.494 g, 14.8 mmol) was added. After 10 min, powder of 4-ethynylsalicylic acid 79 (2.4 g, 14.8 mmol) was added followed by DMF (50 ml). The suspension was cooled in an ice bath. HOBt (0.202 g, 1.5 mmol) and DCC (3.8 g, 18.5 mmol) were added to the reaction mixture at 0° C. The reaction mixture was stirred overnight at RT. The solvent was evaporated in vacuum and under high vacuum to remove DMF. The residue was treated with EA (250 ml) and precipitate of DCU and NEt₃.HCl was filtered (2.5 g). The filtrate was washed with 1N NaHCO₃ (70 ml), water (50 ml), 5% citric acid (50 ml), H₂O (50 ml) and brine (50 ml). The organic solution was dried over Na₂SO₄ and evaporated. The residue (5.8 g) of crude product was purified by chromatography: SiO₂ (100 ml), Hexane:EA 2:1, 1:1, 1:2 to yield 3.0 g, 69.8% yield.

Synthesis of (2R,3S)-2-(4-ethynyl-2-hydroxybenzamido)-3-hydroxybutanoic acid (88): (2R,3S)-methyl 2-(4-ethynyl-2-hydroxybenzamido)-3-hydroxybutanoate 87 (3.2 g, 11.53 mmol) was dissolved in MeOH (120 ml) and a solution of NaOH (1.84 g, 46 mmol) in water (23 ml) was, gradually added with stirring. The reaction mixture was stirred at RT for, 2 h and the MeOH was evaporated in vacuum. The reaction mixture was acidified to pH=7 with 10% citric acid (3.1 g, 15 mmol). EA (350 ml) was added and the solution was acidified to pH=1 with 10% citric acid. The organic layer was washed with water (40 ml) and dried over Na₂CO₃. The solvent was evaporated in vacuum to obtain 3.4 g of solid product. Yield: 98.7%.

Synthesis of ethanaminium (2R,3S)-2-(4-ethynyl-2-hydroxybenzamido)-3-hydroxybutanoate (89): To (2R,3S)-2-(4-ethynyl-2-hydroxybenzamido)-3-hydroxybutanoic acid 88, a solution of H₂NEt (2 ml) in MeOH (100 ml) and the solvent was evaporated in vacuum. CHCl₃ (100 ml) and MeOH (20 ml) were added to the residue and the solvent were evaporated in vacuum. 4.64 g of product was obtained (quantitative yield).

Synthesis of N-((2R,3S)-1-(ethylamino)-3-hydroxy-1-oxobutan-2-yl)-4-ethynyl-2-hydroxybenzamide (90): Ethanaminium (2R,3S)-2-(4-ethynyl-2-hydroxybenzamido)-3-hydroxybutanoate (N-Ethylammonium salt) 89 (4.64 g, 15 mmol) was dissolved in DCM (180 ml) and the solution was cooled in an ice bath. DMF (60 ml) was added to dissolve all the salt and HOBt (202mg, 1.5 mmol) and DCC (3.84 g, 18.8 mmol) were added at 0° C. The reaction mixture was stirred for three days at RT. The solvents were evaporated in high vacuum to remove DMF and the residue was treated with EA (250 ml) and the precipitate of DCU (3.2 g) was removed by filtration. The filtrate was washed with water (40 ml) and brine (40 ml) and dried over Na₂SO₄. The crude product (9 g) was purified by column chromatography: SiO₂ (270 ml), 1% MeOH in CHCl₃, 2% MeOH in CHCl₃ to obtain 1.4 g of yellow solid product. Yield: 41.8%.

Synthesis of (4R,5R)-N-ethyl-2-(4-ethynyl-2-hydroxyphenyl)-5-methyl-4,5-dihydrooxazole-4-carboxamide (91): To a stirred solution of amide 90 (1.4 g, 4.82 mmol) in DCM (36 ml), cooled in an ice bath, SOCl₂ (3.5 ml, 5.75 g, 48.2 mmol) was added. The reaction mixture was stirred overnight at RT. The color of the mixture changed from yellow to black. The reaction mixture was diluted with CHCl₃(45 ml) and evaporated in vacuum. The solid black residue was treated with EA (60 ml) and evaporated. The solid residue dissolved in CHCl₃(200 ml) and dry Na₂CO₃(6 g) was added while stirring after 1 h additional dry Na₂CO₃(6 g) were added. After 3^{rd} addition of dry Na₂CO₃(6 g) and stirring 1 h, the precipitate of Na₂CO₃ was filtered and red filtrate was evaporated in vacuum. The orange solid residue, was purified by column chromatography: SiO₂ (60 ml), CHCl₃, CHCl₃:MeOH (0.5%). 0.84 g, yield: 64.1%. ¹H NMR (300 MHz, CDCl₃) δ ppm 11.59 (s, 1H), 7.63 (d, J=8.10 Hz, 1H), 7.15 (d, J=1.40 Hz, 1H), 7.02 (dd, J=8.11, 1.48 Hz, 1H), 6.44 (s, 1H), 5.24-5.14 (m, 1H), 4.94 (d, J=10.33 Hz, 1H), 3.64-3.23 (m, 2H), 3.20 (s, 1H), 1.40 (d, J=6.50 Hz, 3H), 1.17 (t, J=7.26 Hz, 3H).

### Example 13

### Synthesis of 4-ethynyl L-trans (4R, 5S) POxA Ligand

### Ethynyl L-trans (4R, 5S) (4R,5S)-N-ethyl-2-(4-ethynyl-2-hydroxyphenyl)-5-methyl-4,5-dihydrooxazole-4-carboxamide

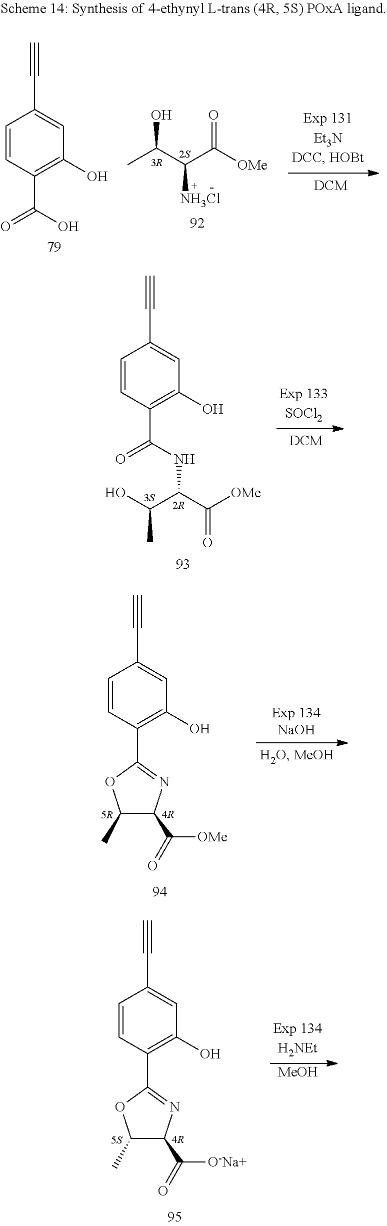

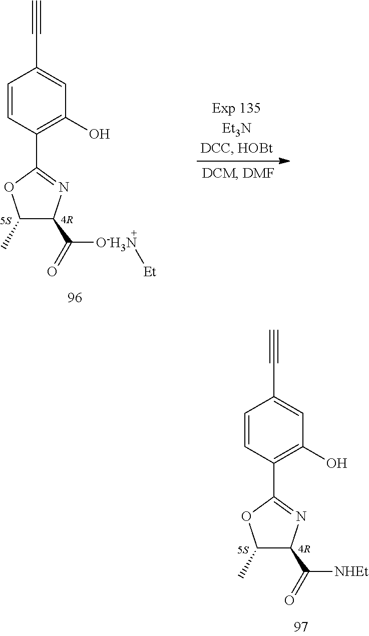

Synthesis of (2S,3R)-methyl 2-(4-ethynyl-2-hydroxybenzamido)-3-hydroxybutanoate (93): To a suspension of L-Threonine methyl ester. HCl 92 (2.17 g, 12.77 mmol) in DCM (165 ml), triethylamine (1.289 g, 12.77 mmol, 1.77 ml) was added and the mixture turned to a clear solution. 4-ethynyl salicylic acid 79 (2.00 g, 12.33 mmol) was added. The reaction mixture was cooled to 0° C. and HOBt (166 mg, 1.233 mmol) and DCC (3.17 g, 15.41 mmol) were added. The reaction mixture was stirred overnight at RT and a precipitate of DCU was formed, which was filtered off (2.76 g). The filtrate was diluted with DCM (50 ml) and washed with water (70 ml), sat. NaHCO3 (70 ml), 5% citric acid (70 ml), water (70 ml) and brine (70 ml). The organic layer was dried over Na2SO4 and evaporated in vacuum. The residue was treated with EA (100 ml) and precipitate was filtered off. The organic solvent was evaporated in vacuum and the residue was purified by flash chromatography: SiO₂ (80 ml), CHCl₃ to obtain 2.2 g of the title compound 93. Yield: 64.7%.

Synthesis of (4R,5R)-methyl 2-(4-ethynyl-2-hydroxyphenyl)-5-methyl-4,5-dihydrooxazole-4-carboxylate (94): (2S,3R)-Methyl 2-(4-ethynyl-2-hydroxybenzamido)-3-hydroxybutanoate 93 (2.2 g, 14.55 mmol) was dissolved DCM (22 ml) and thionyl chloride (9.4 g, 79.3 mmol, 5.8 ml) was added with stirring and cooling in an ice bath. The reaction mixture was stirred overnight at RT. The reaction mixture was diluted with CHCl₃ (10 ml) and evaporated in vacuum. The residue was dissolved in EA (10 ml) and the solution was evaporated (repeated twice). The residue was dissolved in CHCl₃ (100 ml) and dry Na₂CO₃ (6.5 g) was added and the reaction was stirred for 1 h. Dry Na₂CO₃ (6.5 g) was added again and the mixture stirred for 1 h (repeated twice). The precipitate was filtered and the solvent evaporated in vacuum to obtain solid product which was dried in high vacuum to obtain 1.8 g of 94. Yield: 87.5%.

Synthesis of Ethanaminium (4R,5S)-2-(4-ethynyl-2-hydroxyphenyl)-5-methyl-4,5-dihydrooxazole-4-carboxylate (96): (4R,5R)-methyl 2-(4-ethynyl-2-hydroxyphenyl)-5-methyl-4,5-dihydrooxazole-4-carboxylate 94 (1.8 g, 6.94 mmol) was dissolved in MeOH (100 ml) and 2N NaOH (14 ml, 28 mmol) were added dropwise with stirring. The reaction mixture was stirred for 2 h at RT and the solvents were evaporated in vacuum. The residue 95 was dissolved in water (20 ml) and was placed on top of 100 g Amberlite IR-120 column in H₃N⁺Et. The resin column was eluted with water. After evaporation of the water, 2.00 g of 96 was obtained. Yield: 99.3%.

Synthesis of (4R,5S)-N-ethyl-2-(4-ethynyl-2-hydroxyphenyl)-5-methyl-4,5-dihydrooxazole-4-carboxamide (97): Ethanaminium (4R,5S)-2-(4-ethynyl-2-hydroxyphenyl)-5-methyl-4,5-dihydrooxazole-4-carboxylate 96 (2.00 g, 6.89 mmol) was dissolved in DMF (50 ml) and DCM (150 ml) and the solution was cooled in an ice bath. HOBt (93 mg, 0.69 mmol) and DCC (1.77 mg, 8.61 mmol) were added with stirring. The reaction mixture was stirred for 2 days at RT. The reaction mixture was evaporated in vacuum and high vacuum to remove DMF. The residue was dissolved in CHCl₃ (150 ml) and washed with water (70 ml). The organic solvent was evaporated and the residue was dissolved in EA (150 ml) and water (50 ml). The organic layer was washed with water (20 ml) and dried over Na₂SO₄ and evaporated. The residue (3.14 g) of crude product was purified by column chromatography: SiO₂ (70 g), CHCl₃ to obtain 0.97 g of dark orange solid 97. Yield: 46.5%. ¹H NMR (300 MHz, CDCl₃) δ ppm 11.57 (s, 1H), 7.61 (d, J=9.6 Hz, 1H), 7.12 (d, J=1.80 Hz, 1H), 6.99 (dd, J=9.6, 1. 8 Hz, 1H), 6.32(s, 1H), 4.92-4.81 (m, 1H), 4.35 (d, J=9.3 Hz, 1H), 3.41-3.20 (m, 2H), 3.17 (s, 1H), 1.60 (d, J=11.7 Hz, 3H), 1.15 (t, J=6.9 Hz, 3H).

### Example 14

### Synthesis of 5-sulfonate L-cis (4S, 5S) POxA Ligand

### Sodium sulfonate L-cis (4S, 5S) (4R,5S)-N-ethyl-2-(4-ethynyl-2-hydroxy-5-sodium sulfonatephenyl)-5-methyl-4,5-dihydrooxazole-4-carboxamide

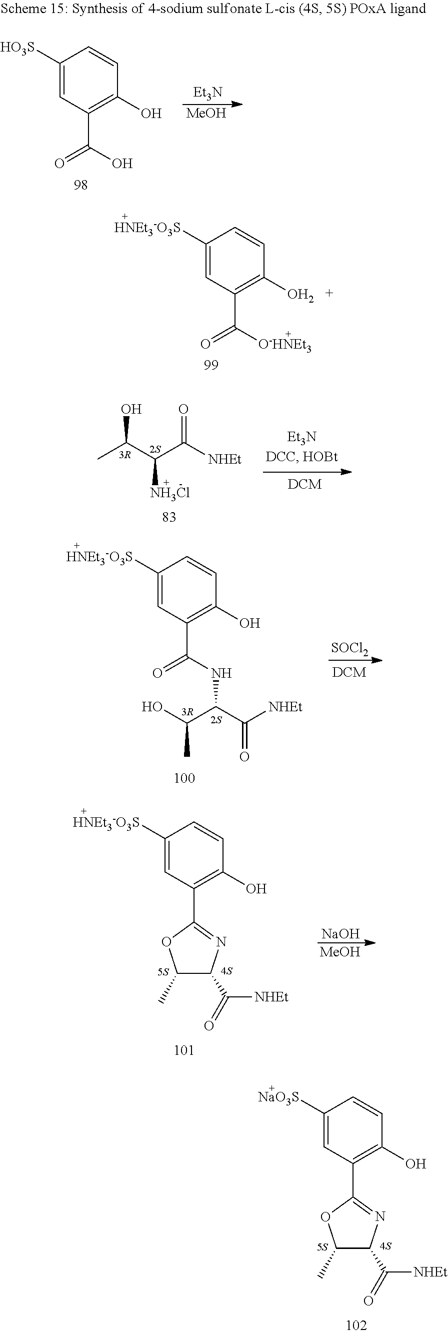

Synthesis of triethylammonium 3-((2S,3R)-1-(ethylamino)-3-hydroxy-1-oxobutan-2-ylcarbamoyl)-4-hydroxybenzenesulfonate (100): To a solution of 5-sulfo-salicylic acid (2-hydroxy-5-sulfobenzoic acid) 98 (0.763 g, 3 mmol) in MeOH (10 ml), triethylamine (0.606 g, 6 mmol, 0.835 ml) was added and the solution was evaporated in vacuum. The residue was dissolved in CHCl₃ (20 ml) and the solution was evaporated in vacuum. The residue of the triethylammonium salt 99 was re-dissolved in CHCl₃ (20 ml) and the solution was added to a solution of L-Thr-N-ethyl amide•HCl 83 (0.56 g, 3.04 mmol) in MeOH (15 ml). The red solution was evaporated, the residue was dissolved in CHCl₃ (30 ml) and triethylamine (0.2 ml) and evaporated to dryness. The residue was dissolved in DCM and dry DMF (3 ml). The mixture was cooled to 0° C. and HOBt (40 mg, 0.3 mmol) and DCC (0.78 g, 3.8 mmol) were added. The reaction mixture was stirred at RT overnight (salt was dissolved completely after 3 h, and then became precipitation of DCU). The precipitate of DCU was filtered (0.32 g) and the filtrate was evaporated in high vacuum to remove DMF. The residue was purified by column chromatography: SiO₂ (60 ml), CHCl₃, CHCl₃:MeOH (64:1), CHCl₃:MeOH (16:1), CHCl₃:MeOH (8:1) and CHCl₃:MeOH (4:1) to obtain 1.09 g of product 100. Yield: 81.3%.

Synthesis of (4R,5S)-N-ethyl-2-(4-ethynyl-2-hydroxy-5-sodium sulfonatephenyl)-5-methyl-4,5-dihydrooxazole-4-carboxamide (102): (2S,3R)-methyl 2-(5-triethylammonium sulfonate-2-hydroxybenzamido)-3-hydroxybutanoate 100 (0.53 g, 1.184 mmol) was suspended in DCM (12 ml) and the mixture was cooled in an ice bath. Thionyl chloride (1.793 g, 15.06 mmol, 1.1 ml) was added at 5° C. The reaction mixture was stirred overnight at RT—a new precipitate was formed. The reaction mixture was diluted with CHCl₃ (25 ml) and evaporated in vacuum (repeated twice). CHCl₃ (30 ml) was added to the residue, followed by Et₃N to pH=9. To this mixture, SiO₂ (2.5 g) was added and the solvents evaporated in vacuum. The mixture was purified by colunm chromatography: SiO₂(15 g), CHCl₃:MeOH (8:1) to obtain the triethylammonium salt 101 (0.6 g). The triethylammonium salt was dissolved in MeOH (15 ml) and solution of NaOH (110 mg) in MeOH (5 ml) was added to pH>12. The solvent was evaporated and the residue dried under high vacuum to obtain the sodium salt 102 (0.42 g). Yield: 101%. ¹H NMR (300 MHz, MeOD-d₄) δ ppm 8.05 (d, J=2.58 Hz, 1H), 7.49 (dd, J=8.88, 2.61 Hz, 1H), 6.61 (d, J=8.87 Hz, 1H), 4.95-4.85 (m, 1H), 4.72 (d, J=10.18 Hz, 1H), 3.21-3.14 (m, 2H), 1.19 (d, J=6.42 Hz, 3H), 1.08 (t, J=7.25 Hz, 3H).

### Example 15

### Synthesis of dimethylamine diazenyl L-cis (4S, 5S) POxA Ligand

### Dimethylamine diazenyl L-cis (4S, 5S) (4S,5S)-2-(4-((E)-(4-(dimethylamino)phenyl)diazenyl)-2-hydroxyphenyl)-N-ethyl-5-methyl-4,5-dihydrooxazole-4-carboxamide

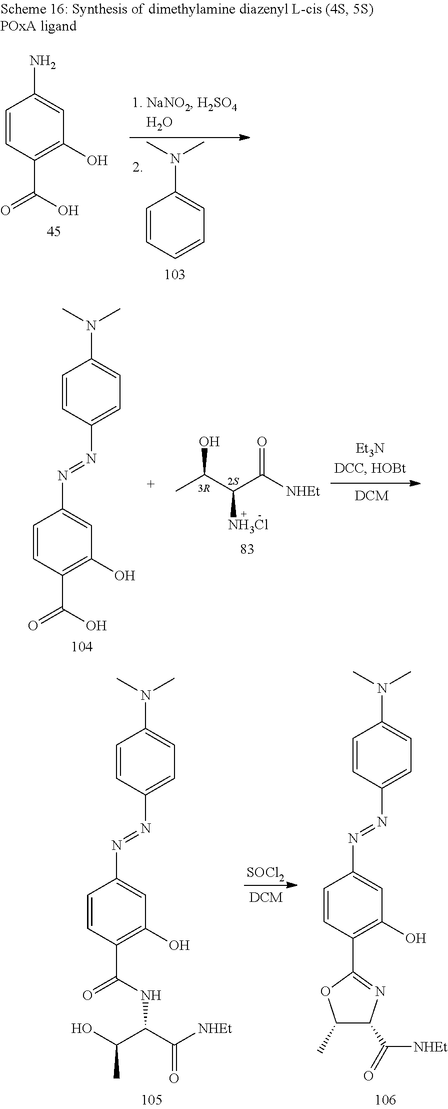

Synthesis of (E)-4-((4-(dimethylamino)phenyl)diazenyl)-2-hydroxybenzoic acid (104): In a three-necked flask, 4-aminosalicylic acid 45 (2.23 g, 14.6 mmol) was mixed with H₂O (13 ml) containing conc. H₂SO₄ (3.65 g, 37.23 mmol) and the mixture was cooled to 0° C.-5° C. with stirring. The mixture was diazotized by gradual addition of cold solution of NaNO₂ (1.0 g, 14.6 mmol) in water (3.5 ml). To the dark suspension was added (at 4° C.) a cold solution of N,N-dimethylaniline 103 (1.77 g, 14.6 mmol) in acetic acid (1.5 ml). This mixture was stirred for 1 h at 5° C. A red plroduct was obtained. After a solution of sodium acetate (6.13 g, 74.5 mmol) was added to the reaction mixture to neutralize the acid, the red precipitate was filtered off and washed twice with water. The filtrate was dried in high vacuum to obtain a dark red solid product 104 (1.2 g). Yield: 28.8%.

Synthesis of 4-((E)-(4-(dimethylamino)phenyl)diazenyl)-N-(2S,3R)-1-(ethylamino)-3-hydroxy-1-oxobutan-2-yl)-2-hydroxybenzamide (105): Triethylamine (0.49 ml, 3.5 mmol) was added to a suspension of L-threonine-N-ethylamide 83 (1.64 g, 3.5 mmol) in CHCl₃ (10 ml). After 10 min, (E)-4-((4-(dimethylamino)phenyl)diazenyl)-2-hydroxybenzoic acid 104 was added, which was not dissolved. The mixture was diluted with MeOH (10 ml) and evaporated in vacuo. The residue was diluted with CHCl3 (20 ml) the suspension was evaporated and dried under high vacuum. The residue was diluted with DCM (80 ml) and cooled in an ice bath. This mixture was diluted with dry DMF (20 ml) and cooled to 0° C. Then, HOBt (47 mg, 0.35 mmol) and DCC (0.9 g, 14.3 mmol) were added and the red reaction mixture was stirred at RT overnight. The solvents were evaporated in high vacuum to remove traces of DMF. The residue was treated with EA (60 ml) and the precipitates of DCU and triethyl ammonium chloride were discarded by filtration. The organic filtrate was washed with H₂O (20 ml), dried over Na₂SO₄ and evaporated. The raw product was purified by column chromatography: SiO₂(100 ml), CHCl₃, CHCl₃:MeOH (1.5%) to obtain the red solid product 105 (0.9 g). Yield: 62.2%.

Synthesis of (4S,5S)-2-(4-((E)-(4-(dimethylamino)phenyl)diazenyl)-2-hydroxyphenyl)-N-ethyl-5-methyl-4,5-dihydrooxazole-4-carboxamide (106): A suspension of 4-((E)-(4-(dimethylamino)phenyl)diazenyl)-N-(2S,3R)-1-(ethylamino)-3-hydroxy-1-oxobutan-2-yl)-2-hydroxybenzamide 105 (0.8 g, 2 mmol) in DCM (52 ml) was gradually added with cooling to SOCl₂ (2.2 ml, 30.1 mmol). The reaction mixture was stirred at RT overnight. A dark blue-red solution wasc formed, with dark precipitate on the walls of the flask. Reaction mixture was diluted with CHCl₃ and was evaporated in vacuo (repeated 3 times). The residue was dissolved in dry DMF (50 ml) and NaCO₃ (5 g) were added and the mixture was stirred for 1 h. The mixture was diluted with CHCl₃ (70 ml) and stirred for additional 1 h. The mixture was filtered and the filtrate was evaporated in vacuo and dried under high vacuum. The red solid was purified by column chromatography: SiO₂(20 ml), CHCl₃, CHCl₃:MeOH (0.5%) to obtain the red solid product 106 (0.55 g). Yield: 71.9%. ¹H NMR (300 MHz, CDCl₃) δ ppm 7.85 (d, J=8.90 Hz, 2H), 7.70 (d, J=8.41 Hz, 1H), 7.40 (d, J=1.76 Hz, 1H), 7.34 (dd, J=8.40, 1.80 Hz, 1H), 6.69 (d, J=9.32 Hz, 2H), 6.45 (t, J=5.11, 5.11 Hz, 1H), 5.13 (qd, J=10.29, 6.50, 6.50, 6.50 Hz, 1H), 4.88 (d, J=10.31 Hz, 1H), 3.53-3.10 (m, 2H), 3.04 (s, 6H), 1.34 (d, J=6.52 Hz, 3H), 1.10 (t, J=7.25, 7.25 Hz, 3H).

### Example 16

### Synthesis of dimethylamine dialkynyl L-cis (4S, 5S) POxA Ligand

### Dimethylamine alkynyl L-cis (4S, 5S) (4S,5S)-2-(4-((4-(dimethylamino)phenyl)ethynyl)-2-hydroxyphenyl)-N-ethyl-5-methyl-4,5-dihydrooxazole-4-carboxamide

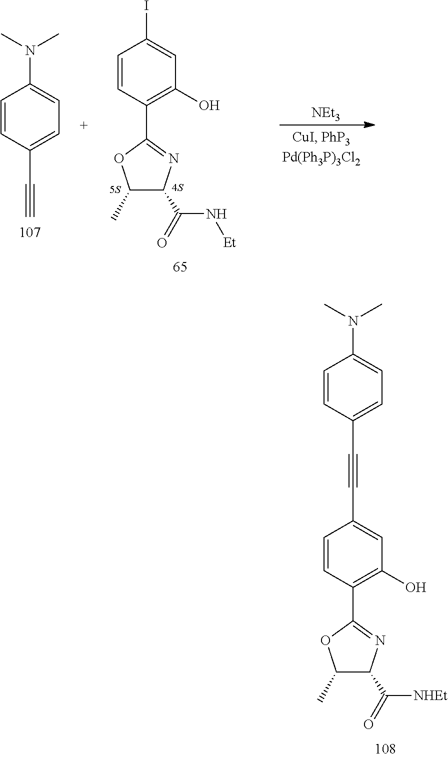

Synthesis of (4S,5S)-2-(4-((4-dimethyamino)phenyl)ethynyl)-2-hydroxyphenyl)-N-ethyl-5-methyl-4,5-dihydrooxazole-4-carboxamide (108): A suspension of 4-ethynyl-N,N-dimethylaniline 107 (57 mg, 0.392 mmol) and (4S,5S)-N-ethyl-2-(2-hydroxy-4-iodophenyl)-5-methyl-4,5-dihydrooxazole-4-carboxamide 65 (123 mg, 0.328 mmol) in triethylamine (2 ml), was stirred under nitrogen atmosphere. CuI (1 mg) Ph₃P (2 mg) and Pd(Ph₃P)₃Cl₂ (1 mg) were added and the mixture was stirred under reflux for 3 h. After cooling, the reaction mixture was treated with EA (15 ml) and water (5 ml). The layers were separated and the dark organic solution was washed with H₂O (2 ml) and dried over Na₂SO₄ and evaporated in vacuo. The residue was diluted with EA and evaporated again to obtain 0.15 g of dark yellow solid, which was purified by column chromatography: SiO₂(3 g), CHCl₃, CHCl₃:MeOH (0.5%) to obtain the yellow product 108 (84 mg). Yield: 65%. ¹H NMR (300 MHz, CDCl₃) δ ppm 11.50 (s, 1H), 7.57 (d, J=8.16 Hz, 1H), 7.37 (d, J=8.89 Hz, 2H), 7.08 (d, J=1.40 Hz, 1H), 6.97 (dd, J=8.14, 1.49 Hz, 1H), 6.63 (d, J=8.64 Hz, 2H), 6.42 (t, J=5.25, 5.25 Hz, 1H), 5.12 (qd, J=10.27, 6.50, 6.50, 6.50 Hz, 1H), 4.87 (d, J=10.29 Hz, 1H), 3.45-3.12 (m, 2H), 2.95 (s, 6H), 1.34 (d, J=6.51 Hz, 3H), 1.11 (t, J=7.25, 7.25 Hz, 3H).

### Example 17

### Synthesis of (phenylethynyl)phenyl L-cis (4S, 5S) POxA Ligand

### (phenylethynyl)phenyl L-cis (4S, 5S) (4S,5S)-N-ethyl-2-(2-hydroxy-4-(phenylethynyl)phenyl)-5-methyl-4,5-dihydrooxazole-4-carboxamide

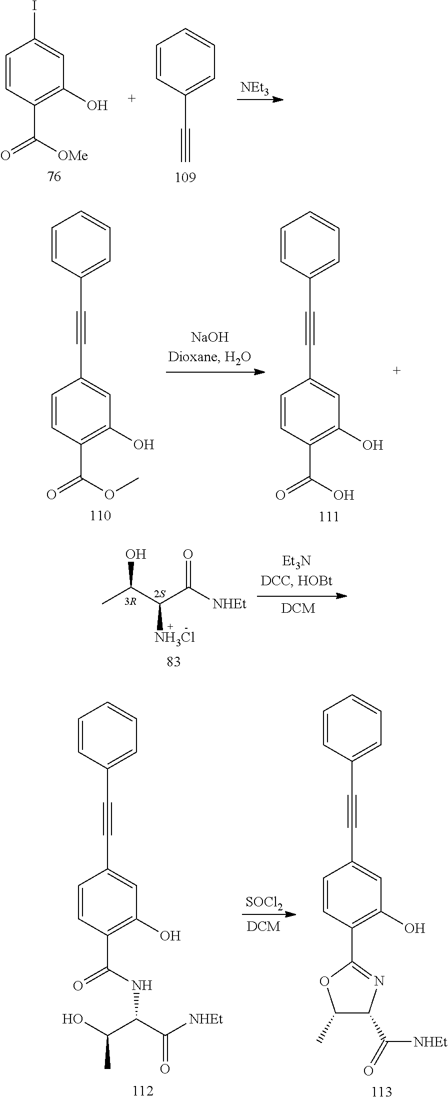

Synthesis of methyl 2-hydroxy-4-(phenylethynyl)benzoate (110): A solution of methyl 4-iodosalicylate 76 (0.834 g, 3 mmol) and ethynylbenzen 109 (0.395 ml, 0.368 g, 3.6 mmol) in NEt₃ (9 ml) was prepared under N₂. CuI (3 mg), PPh₃ (6 mg) and Pd(PPh₃)Cl₂ (3 mg) were added. The mixture was stirred under at 80°-90° for 0.5 h. NEt₃.HI was precipitated. The reaction mixture was cooled and 70 ml of EA and 250 ml water were added. The organic solution was separated washed with 12 ml of water and dried over Na₂SO₄. The organic solution was evaporated to obtain 1.1 g of light brown solid product, which was purified by column chromatography: SiO₂(20 g), Hexane; Hexane:CHCl₃ (20:1), Hexane:CHCl₃ (4:1), to obtain 0.707 g of solid colorless product. Yield: 94.26%.

Synthesis of 2-hydroxy-4-(phenylethynyl)benzoic acid (111): A mixture of methyl 2-hydroxy-4-(phenylethynyl)benzoate 110 (0.7 g, 2.77 mmol), NaOH (2.5 g, 27.7 mmol) in water (15 ml) and dioxane (50 ml) was refluxed for 4 h. Two layers were observed. The reaction mixture was evaporated in vacuo to remove dioxane. 5% citric acid (70 ml) were added to the aqueous solution to pH 3-4 and a suspension was formed. EA (75 ml) were added and the organic layer was separated. The organic layer was washed with water (10 ml) and dried over Na₂SO₄. The organic solvent was evaporated in vacuo to obtain the desired product 111 (0.65 g). Yield: 98.6%.

Synthesis of N-((2S,3R)-1-(ethylamino)-3-hydroxy-1-oxobutan-2-yl)-2-hydroxy-4-(phenylethynyl)benzamide (112): To a suspension of L-threonine-N-ethylamide hydrochloride salt 83 (0.503 g, 2.757 mmol) in CHCl₃ (40 ml), triethylamine (0.278 ml, 2.757 mmol) and 2-hydroxy-4-(phenylethynyl)benzoic acid 111 were added with stirring. A dark solution was formed, and the solvent was evaporated. The residue was dissolved in DCM (65 ml) and cooled in an ice bath. HOBt (37.5 mg, 0.265 mmol) and DCC (0.682 g, 3.31 mmol) were added and the reaction mixture was stirred at RT overnight, but the starting compound was not consumed. The reaction mixture was cooled in an ice bath and additional HOBt (40 mg, 0.28 mmol) and DCC (0.69 mg, 3.31 mmol) were added. After stirred for 2 days, the reaction mixture was evaporated and the residue was treated with EA (60 ml). The precipitated DCU and Net₃ HCl were filtered off. The organic filtrate was washed with H₂O (20 ml), dried over Na₂SO₄ and evaporated. The raw product was purified by column chromatography: SiO₂(80 ml), CHCl₃, CHCl₃:MeOH (1.5%) to obtain the red solid product 112 (0.37 g). Yield: 38.1%.

Synthesis of (4S,5R)-N-ethyl-2-(2-hydroxy-4-(phenylethynyl)phenyl)-5-methyl-4,5-dihydrooxazole-4-carboxamide (113): N-((2S,3R)-1-(ethylamino)-3-hydroxy-1-oxobutan-2-yl)-2-hydroxy-4-(phenylethynyl)benzamide 112 (0.37 g, 1 mmol) was dissolved in DCM (10 ml) and the mixture was cooled in an ice bath. Thionyl chloride (0.73 ml, 1.189 g, 10 mmol) was added and the reaction mixture was stirred at RT. A yellow solution was formed, and a precipitate was formed after 3 h. After stirring overnight, the reaction mixture was diluted with CHCl₃ (20 ml) and evaporated in vacuum. The residue was suspended in EA (30 ml) and evaporated. The residue was treated with CHCl₃ (60 ml) and dry Na₂CO₃ was added. After 1 h, a second portion of dry Na₂CO₃ was added to obtain pH>7. MeOH (2 ml) was added and the mixture was filtered. The filtrate was evaporated in vacuo and the residue was purified by column chromatography: SiO₂(8 g), CHCl₃, CHCl₃:MeOH (0.5%) to obtain compound 113 (0.278 g). Yield: 79.9%. ¹H NMR (300 MHz, CDCl₃) δ ppm 7.60 (d, J=8.14 Hz, 1H), 7.51-7.45 (m, 2H), 7.34-7.26 (m, 3H), 7.13 (d, J=1.38 Hz, 1H), 7.00 (dd, J=8.13, 1.49 Hz, 1H), 6.45 (s, 1H), 5.14 (qd, J=10.34, 6.49, 6.49, 6.48 Hz, 1H), 4.90 (d, J=10.32 Hz, 1H), 3.50-3.11 (m, 2H), 1.34 (d, J=6.49 Hz, 3H), 1.11 (t, J=7.25, 7.25 Hz, 3H).

### Example 18

### Synthesis of L-cis (4S, 5S) POx morpholine amide Ligand ((4S,5S)-2-(2-hydroxyphenyl)-5-methyl-4,5-dihydrooxazol-4-yl)(morpholino)methanone

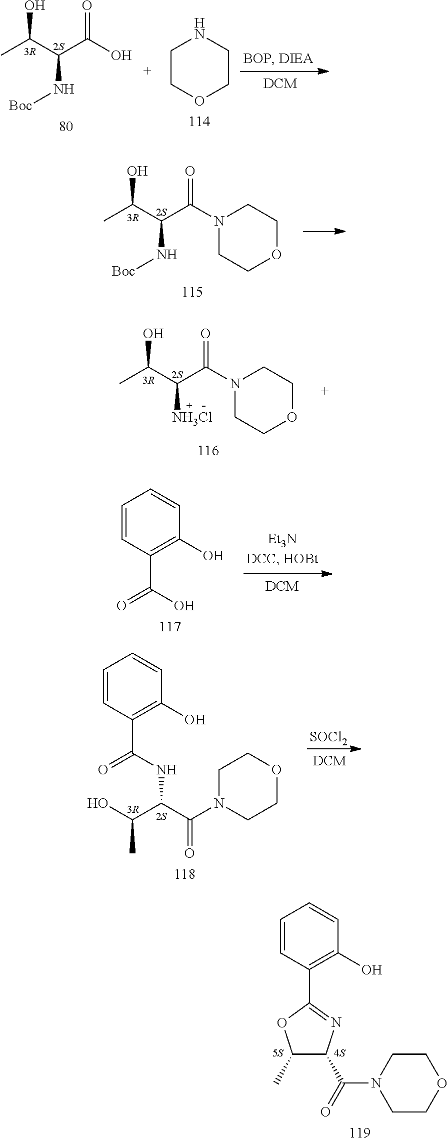

Synthesis of tert-butyl ((2S,3R)-3-hydroxy-1-morpholino-1-oxobutan-2-yl)carbamate (115): Boc-Thr-OH 80 (1.096 mg, 5 mmol) was dissolved in DCM (50 ml). To the stirred solution, BOP (2.21 g, 5 mmol), DIEA (1.72 ml, 10 mmol) and morpholine 114 (0.478 ml, 5.5 mmol) were added. After 20 min of stirring, the reaction solution was concentrated in vacuo, and EA (50 ml) was added. The organic layer was washed with acidic, basic and neutral aqueous solutions, dried over Na₂SO₄ and concentrated in vacuo. The residue was purified by column chromatography: SiO₂(50 g), CHCl₃:MeOH (1%) to obtain compound 115 (1.24 g). Yield: 86%.

Synthesis of (2S,3R)-3-hydroxy-1-morpholino-1-oxobutan-2-aminium chloride (116): Solution of tert-butyl ((2S,3R)-3-hydroxy-1-morpholino-1-oxobutan-2-yl)carbamate 115 (1.24 g, 4.3 mmol) in MeOH (12 ml) was cooled in an ice bath and 4N HCl in dioxane (4 ml) was added with stirring. The reaction mixture was stirred at 5° C. for 30 min and then at RT for 30 min. The reaction solution was evaporated and the residue was dissolved in MeOH (10 ml) and the new solution was evaporated again. The residue was dried under vacuum to obtain 0.26 g of solid product 116. Yield: 100%.

Synthesis of 2-hydroxy-N-((2S,3R)-3-hydroxy-1-morpholino-1-oxobutan-2-yl)benzamide (118): (2S,3R)-3-hydroxy-1-morpholino-1-oxobutan-2-aminium chloride 116 (0.96 g, 4.284 mmol) was dissolved in DCM (100 ml) and Net₃ (0.596 ml, 4.284 mmol) was added. The solution was stirred for 10 min and salicylic acid 117 (0.57 g, 4.427 mmol) was added. The reaction solution was cooled to 0° C. and HOBt (55.7 mg, 0.4127 mmol) and DCC (1.06 g, 5.158 mmol) were added and the reaction mixture was stirred overnight. The precipitate of DCU was filtered off and the residue was evaporated in vacuo. The residue was treated with EA (50 ml) and the precipitate of triethylammonium chloride was filtered off. The organic filtrate was washed with H₂O (10 ml), 1N NaHCO₃ (20 ml), 5% citric acid (20 ml) and brine (20 ml). After drying over Na₂SO₄ the organic solution was evaporated to obtain 1.5 g of yellow oil. The residue was purified by column chromatography: SiO₂(30 g), CHCl₃, CHCl₃:MeOH (1%) to obtain compound 118 (0.8 g). Yield: 70%.

Synthesis of ((4S,5S)-2-(2-hydroxyphenyl)5-methyl-4,5-dihydrooxazole-4-yl)(morpholino)methanone (119): 2-hydroxy-N-((2S,3R)-3-hydroxy-1-morpholino-1-oxobutan-2-yl)benzamide 118 (0.8 g, 2.59 mmol) was dissolved in DCM (20 ml) and the mixture was cooled in an ice bath. Thionyl chloride (3.2 ml, 5.2 g, 43.8 mmol) was added and the reaction mixture was stirred at RT. A suspension was formed, which was diluted with CHCl₃ (20 ml) and evaporated in vacuum. The residue was suspended in EA (20 ml) and evaporated. The residue was treated with CHCl₃ (250 ml) and dry Na₂CO₃ (1.7 g) was added and stirred for 1 h. After 1 h, a second portion of dry Na₂CO₃ was added to obtain pH>7. The solids were filtered off and the yellow filtrate was evaporated. The solid product (˜1 g) was purified by column chromatography: SiO₂(20 g), CHCl₃, CHCl₃:MeOH (0.5%) to obtain compound 119 (0.3 g). Yield: 39.9%. ¹H NMR (300 MHz, CDCl₃) δ ppm 7.58 (dd, J=7.84, 1.63 Hz, 1H), 7.31 (t, J=8.43, 8.43 Hz, 1H), 6.94 (d, J=8.33 Hz, 1H), 6.80 (t, J=7.56, 7.56 Hz, 1H), 5.19 (d, J=9.87 Hz, 1H), 4.95 (qd, J=9.99, 6.55, 6.54, 6.54 Hz, 1H), 3.82-3.28 (m, 8H), 1.35 (d, J=6.46 Hz, 3H).

### Example 19

### Synthesis of tetraacetyl amido L-cis (4S, 5S) POxA Ligand

### Synthesis of Phenyl oxazole acid L-cis (4S, 5S)

### (4S,5S)-2-(2-hydroxyphenyl)-5-methyl-4,5-dihydrooxazole-4-carboxylic acid

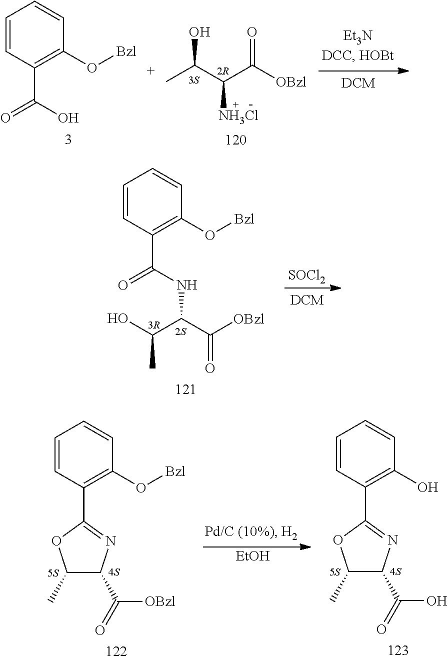

Synthesis of (2S,3R)-benzyl 2-(2-(benzyloxy)benzamido)-3-hydroxybutanone (121): To a solution of Ba(OH)₂.8H₂O (3.3 g, 10.5 mmol) in H₂O (20 ml), 5N HCl (21 mmol) was added to pH=2. The solution of BaCl₂ was added to a suspension of L-threonine benzyl ester oxalate (3.14 g, 10.5 mmol) in water (10 ml) with stirring. After 0.5 h, the precipitate of Ba(CO₂⁻)₂ was filtered off, washed with waster (5 ml) and the filtrate was evaporated with MeOH (100 ml). The residue was diluted with MeOH and evaporated again. The residue was diluted with CHCl₃, evaporated and dried in high vacuum to obtain L-threonine benzyl ester•HCl 120. 120 was dissolved in DCM (180 ml) and NEt₃ (1.06 g, 10.5 mmol) was added with stirring. After 10 min, 2-(benzyloxy)benzoic acid 3 (2.28 g, 10 mmol) was added, and the solution was cooled in an ice bath. HOBt (0.135 g, 0.1 mmol), and DCC (2.57 g, 12.5 mmol) were added and the reaction mixture was stirred overnight. The precipitate od DCU was filtered off and the filtrate was evaporated in vacuo. The residue was treated with EA (200 ml) and precipitate was filtered off. The filtrate was washed with H₂O (50 ml), 1N NaHCO₃ (50 ml), 5% citric acid (50 ml), H₂O (50 ml) and brine (50 ml). The organic layer was dried over Na₂SO₄ and evaporated. The residue was purified by column chromatography: SiO₂(100 g), CHCl₃, CHCl₃:MeOH (1.5%) to obtain compound 121 (3.65 g). Yield: 87%.

Synthesis of (4S,5S)-benzyl 2-(2-(benzyloxy)phenyl)-5-methyl-4,5-dihydrooxazole-4-carboxylate (122): (2S,3R)-benzyl 2-(2-(benzyloxy)benzamido)-3-hydroxybutanone 121 (4.12 g, 10 mmol) was dissolved in DCM (70 ml) and the mixture was cooled in an ice bath. Thionyl chloride (7.3 ml, 11.9 g, 100 mmol) was added and the reaction mixture was stirred at RT overnight. The solution was diluted with CHCl₃ (15 ml) and evaporated in vacuum. The residue was suspended in EA (15 ml) and evaporated. The residue was treated with CHCl₃ (70 ml) and dry Na₂CO₃ (10 g) was added and stirred for 1 h. After 1 h, a second portion of dry Na₂CO₃ was added and the mixture was stirred overnight. The solids were filtered off and the filtrate was evaporated. The solid product (3.9 g) was purified by column chromatography: SiO₂(60 g), hexane:EA (4:1) to obtain compound 122 (2.9 g). Yield: 72.3%.

Synthesis of (4S,5S)-2-(2-hydroxyphenyl)-5-methyl-4,5-dihydrooxazole-4-carboxylic acid (123): (4S,5S)-benzyl 2-(2-(benzyloxy)phenyl)-5-methyl-4,5-dihydrooxazole-4-carboxylate 122 (1.4 g, 3.487 mmol) was suspended in EtOH (200 ml) and MeOH (50 ml), and 10% Pd/C (0.42 g) were added. The reaction mixture was stirred under hydrogen atmosphere at 1 atm for 3 h. The reaction mixture was filtered and the filtrate was evaporated to obtain 123 as solid (0.78 g). Yield: 100%. ¹H NMR (300 MHz, CDCl₃) δ ppm 7.64 (dd, J=7.90, 1.50 Hz, 1H), 7.40 (t, J=8.54, 8.54 Hz, 1H), 7.04 (d, J=8.32 Hz, 1H), 6.86 (t, J=7.54, 7.54 Hz, 1H), 5.20 (td, J=16.18, 6.06, 6.06 Hz, 1H), 5.09 (d, J=10.20 Hz, 1H), 1.52 (d, J=6.31 Hz, 1H).

### Synthesis of Tetraacetylaminoglucose Hydrochloride

### Tetraacetylaminoglucose hydrochloride (2R,3R,4R,5S,6R)-2,4,5-triacetoxy-6-(acetoxymethyl)tetrahydro-2H-pyran-3-aminium

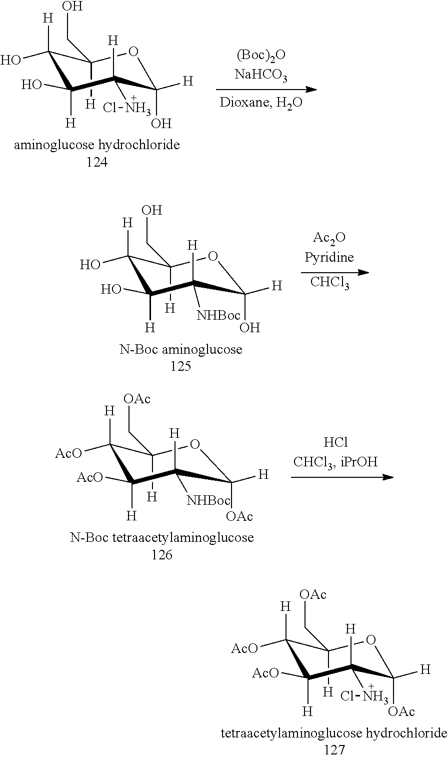

Synthesis of tert-butyl ((2S,3R,4R,5S,6R)-2,4,5-trihydroxy-6-(hydroxymethyl)tetrahydro-2H-pyran-3-yl)carbamate (125): (2S,3R,4R,5S,6R)-2,4,5-trihydroxy-6-(hydroxymethyl)tetrahydro-2H-pyran-3-aminium chloride (glucosamine•HCl) 124 (2.15 g, 10 mmol) was dissolved in a mixture of dioxane (15 ml) and H₂O (15 ml). The solution was cooled in an ice bath and NaHCO₃ (2.48 g, 29.5 mmol) and (Boc)₂O (2.42 g, 11.3 mmol) were added. The reaction was stirred for 15 min at 0° C. and then at RT for 2 days (a precipitate was formed after 3 h). The reaction mixture was diluted with EtOH (30 ml) and the solvents were evaporated in vacuo. The residue was dried under high vacuum to obtain a mixture of N-Boc-amino glucose and salts formed in the reaction. The mixture was used in the next reaction without purification.

Synthesis of (2R,3R,4R,5S,6R)-6-(acetoxymethyl)-3-((tert-butoxycarbonyl)amino)tetrahydro-2H-pyran-2,4,5-triyl triacetate (126): To the above mixture (10 mmol) in CHCl₃, pyridine (19.6 g, 247 mmol) were added and the mixture was cooled in an ice bath. Ac₂O (21.6 g, 211 mmol) was added and the solids were completely dissolved after 2 h at RT. The reaction mixture was stirred at RT overnight. After 18 h, the reaction mixture was added to a mixture of 40 g ice and CHCl₃ (40 ml). The organic layer was separated, washed with H₂O (20 ml) and evaporated in vacuo. The residue was dissolved in diethyl ether (50 ml) and the organic solution was washed with H₂O (20 ml), 3 times with 0.25N NaHSO₄ (20 ml) and H₂O (20 ml). The organic layer was dried over Na₂SO₄ and evaporated in vacuo. The, residue was diluted with CHCl₃ (30 ml) and the solution was evaporated in vacuo. The new residue was dried under high vacuum to obtain 126 (4.4 g). Yield: 100%.

Synthesis of (2R,3R,4R,5S,6R)-2,4,5-triacetoxy-6-(acetoxymethyl)tetrahydro-2H-pyran-3-aminium (127): (2R,3R,4R,5S,6R)-6-(acetoxymethyl)-3-((tert-butoxycarbonyl)amino) tetrahydro-2H-pyran-2,4,5-triyl triacetate 126 (4.4 g, 10 mmol) was dissolved in CHCl₃ (10 ml) and the solution was cooled in an ice bath. 4N HCl in dioxane (15 ml) was added, followed by dry iPrOH (10 ml). The reaction mixture was stirred for 3 h at RT. A large amount of precipitate was observed. Reaction mixture was diluted with CHCl₃ (30 ml) and evaporated in vacuo. The residue was diluted with CHCl₃ (60 ml) to obtain a cloudy solution, which was evaporated and the residue was dried in high vacuum to obtain a solid product (2R,3R,4R,5S,6R)-2,4,5-triacetoxy-6-(acetoxymethyl)tetrahydro-2H-pyran-3-aminium 127 (3.6 g). Yield: 94%. ¹H NMR (300 MHz, CDCl₃) δ ppm 8.86 (s, 1H), 6.53 (d, J=3.28 Hz, 1H), 5.4 (m, 1H), 5.1 (m, 1H), 4.27 (dd, J=13.10, 4.57 Hz, 1H), 4.07 (m, 1H), 3.82-3.58 (m, 2H), 2.12 (m, 12H).

### Synthesis of tetraacetyl amido L-cis (4S, 5S) POxA Ligand

### (4S,5S)-2-(2-hydroxyphenyl)-5-methyl-4,5-dihydrooxazole-4-amidoglucose

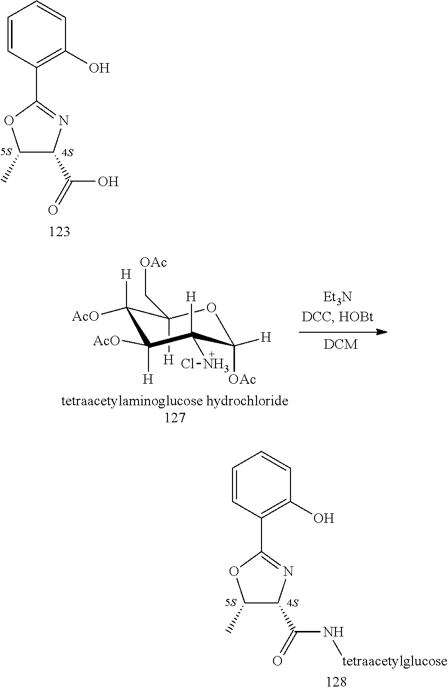

Synthesis of 6-(acetoxymethyl)-3-((4S,5S)-2-(2-hydroxyphenyl)-5-methyl-4,5-dihydrooxazole-4-carboxamido)tetrahydro-2H-pyran-2,4,5-triyl triacetate (128): Et₃N (0.37 g, 3.67 mmol) was added to a suspension of (2R,3R,4R,5S,6R)-2,4,5-triacetoxy-6-(acetoxymethyl)tetrahydro-2H-pyran-3-aminium 127 (1.409 g, 3.67 mmol) in CHCl₃ (50 ml). A solution of (4S,5S)-2-(2-hydroxyphenyl)-5-methyl-4,5-dihydrooxazole-4-carboxylic acid 123 (0.78 g, 3.53 mmol) in DCM (50 ml) was added and the reaction mixture was cooled to −2° C. with stirring. HOBt (0.047 g, 0.35 mmol) and DCC (0.91 g, 4.41 mmol) were added and the solution was stirred overnight at RT. Precipitate of DCU was observed after 2 h. The precipitate was filtered off, and the filtrate was evaporated in vacuo. The residue was treated with EA (100 ml) and the new precipitate was filtered off. The filtrate was washed with H₂O (15 ml), 1N NaHCO₃ (15 ml), 5% citric acid (15 ml), H₂O (15 ml) and brine (15 ml). The organic layer was dried over Na₂SO₄ and evaporated in vacuo to obtain 4.8 g of crude product, which was purified by column chromatography: SiO₂(60 g), hexane:EA (4:1), hexane:EA (2:1) to obtain compound 128 (0.95 g). Yield: 48.96%. ¹H NMR (300 MHz, CDCl₃) δ ppm 7.69 (d, J=7.90, Hz, 1H), 7.44 (t, J=6.97, 6.97 1H), 7.08 (d, J=8.74 Hz, 1H), 6.92 (t, J=7.05, 7.05 Hz, 1H), 6.26 (d, J=3.78 Hz, 1H), 5.28 (m, 2H), 5.14 (td, J=12.96, 4.48, 4.48 Hz, 1H), 4.89 (d, J=10.08 Hz, 1H), 4.39 (m, 1H), 4.26 (dd, J=12.53, 4.05 Hz, 1H), 4.07 (dd, J=12.50, 2.34 Hz, 1H), 3.99 (ddd, J=9.63, 3.75, 2.31 Hz, 1H), 2.13 (s, 3H), 2.09 (s, 3H), 2.05 (s, 3H), 1.99 (s, 3H), 1.29 (d, J=6.49 Hz, 1H).

### Example 20

### Synthesis of Lanthanide Clusters of this Invention

Multi-lanthanide complexes were prepared by mixing the ligand with LiOH and subsequent addition of LnCl₃. Two distinct types of clusters were obtained, depending on the geometrical isomer used as ligand for the complexation. The cis derivatives form the 3-Ln clusters while the trans form 7-Ln clusters (FIG. 2).

A ligand of this invention (100 mg, 0.4 mmol) and lithium hydroxide (9 mg, 0.4 mmol, not completely dissolves) were stirred in methanol (3 ml) for 30 min. at room temperature. To this mixture lanthanum chloride (68 mg, 0.18 mmol) solution in 2 ml of methanol was added drop-wise and stirred for 15 minutes. The solution was filtrated (0.45 uM filter) and allowed to evaporate slowly for several days to form crystals.

Synthesis of the 3Tb cluster ([Tb₃(L)₆(μ₃-OH)(MeOH)₃]Cl₂(MeOH)₃ (LiOH)(H₂O)₂Cluster):

Ligand cis-(4S,5S)-POxA (100 mg, 0.4 mmol) and lithium hydroxide (9 mg, 0.4 mmol, not completely dissolves) were stirred in methanol (3 ml) for half a hour at room temperature. To this mixture terbium chloride (68 mg, 0.18 mmol) solution in 2 ml of methanol was added drop-wise and the resultant solution was allowed to evaporate slowly for several days. White needles were filtered out and washed with methanol (yield: 75%). Maldi-MS: 986.81 [Tb₃L₆(μ₃-OH)]²⁺, 1018.82[Tb₃L₆(μ₃-OH)MeOH]²⁺. selected atomic distances [Å]: Tb(1)-O(0) 2.374(8); Tb(1)-O(1) 2.355(8); Tb(1)-O(61) 2.392(8); Tb(1)-O(4) 2.422(8); Tb(1)-O(41) 2.451(8); Tb(1)-O(44) 2.505(9); Tb(1)-Tb(2) 3.9166(11); Tb(1)-Tb(3) 3.9281(12); Selected bond angles: Tb(10)-O(0)-Tb(2) 111.0(3); O(1)-Tb(1)-O(0) 79.8(3); O(1)-Tb(1)-O(4) 133.4(3). The structure of 3Tb cluster is presented in FIG. 2.

The same method was employed for the preparation of trinuclear Sm³⁺, Pr³⁺, Gd³⁺ and La³⁺ clusters.

Under similar conditions the enantiomer cis-(4R,5R)-phenol oxazoline ethyl amide provides enantiomeric trinuclear Tb³⁺(3Tb) and trinuclear Gd³⁺ clusters. The two isostructural clusters, the luminescent tri-Tb³⁺, and the tri-Gd³⁺, possess opposite chirality (see CD in FIG. 6). Formation of all tri-lanthanide 3-Ln cluster complexes was confirmed by MS-MALDI spectroscopy, and selected complexes were subjected to crystallographic analysis, and CD measurements (FIG. 6).

Identical conditions, starting with the trans POxA ligands provided the 7Ln clusters providing opposite chirality when starting with trans-(4S-5R) POxA ligand or trans-(4R-5S) POxA ligand as observed in CD measurements.

3-Ln Cluster Structure

Single crystal X-ray diffraction analysis showed that the orthorhombic crystal belongs to chiral P2₁2₁2₁ space group, with a single molecular complex comprising the asymmetric unit. Anomalous scattering method was applied to establish the crystal absolute configuration.

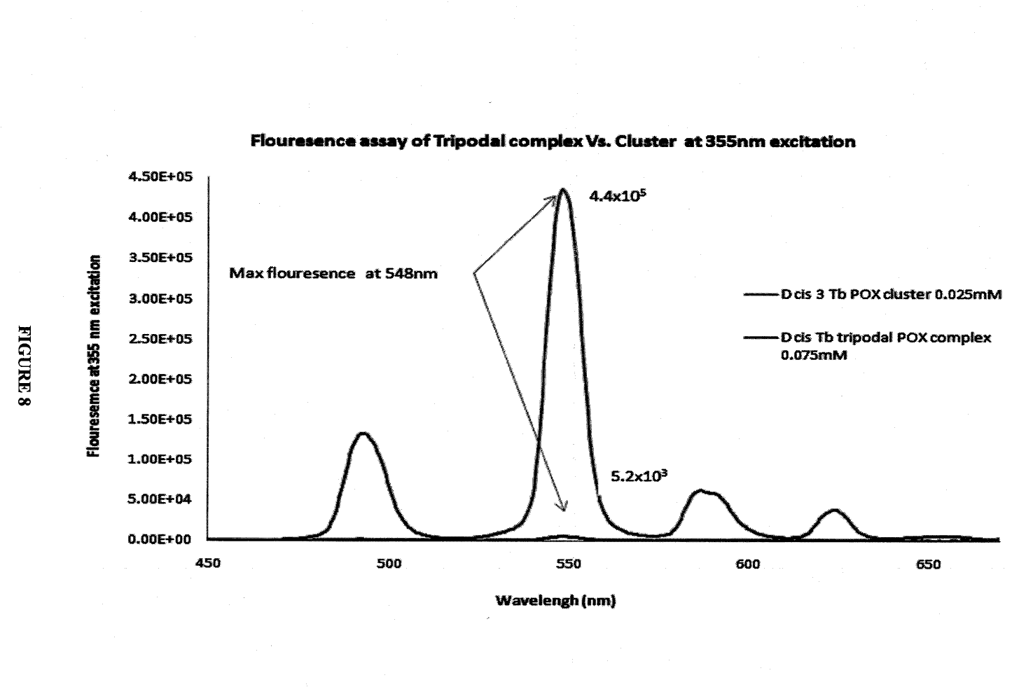

The six ligands in 3-Tb cluster form a left handed chiral ‘barrel’ hosting tri-Tb ions at its center. The metal core is associated with a μ₃-OH lying out of the metal plane. The ligand orientation alternates in an anti-parallel manner, such that the aliphatic moiety of one is pointing toward the aromatic domain of the neighboring ligand. Each Tb³⁺ ion is coordinated by two tridentate (ONO) ligands and a methanol molecule, resembling a monocapped square antiprism geometry (FIG. 7). The methanol molecules are enclosed within the ‘barrel’ adopting a left-handed (A) orientation.

Enantiomeric, tri-Gd³⁺ cluster grows in triclinic crystals belonging to the P1 space group as was revealed by X-ray diffraction analysis, with two molecules in the asymmetric unit. All other parameters were found similar, yet mirror images, to the crystal.

7Ln Cluster Structure

Single crystal X-ray diffraction analysis showed that the space group belogs to chiral crystalographic domain.

The 7Ln clusters demonstrated distinct chiral entities composed of 9 ligands encapsulating a multi-nuclear lanthanide core generated by extensive network of oxygen bridges (six μ₃-oxo bridges) between a central and six periferial lanthanide ions (FIG. 2A).

Table 1 presents chracterization of selected clusters of this invention.

TABLE 1
Summery of selected X-ray structures and crystal characteristics.

| | 4,iodo L-cis | D-cis 3La | L-cis 3La | 4,Azido D- |
|---|---|---|---|---|
| | 3La POxA | POxA | POxA | cis POxA |
| Complex | cluster | cluster | cluster | cluster |
| Asymmetric | C₇₈H₇₈I₆La₃ | C₈₁H₉₅La₃ | C₈₀H₉₈La₃N₁₂ | C₈₁H₇₈La₃N₃₀ |
| unit content | N₁₂O₂₂ | N₁₂O₂₂, | O₂₂, + 2.33 | O₂₅, + Cl + O |
| | | | (CH4O) + | |
| | | | 2Cl | |
| Crystal | Colourless | Colourless | Colourless | Yellow |
| description | prism | needle | needle | pyramid |
| Crystal size | 0.12 × 0.03 × | 0.24 × 0.11 × | 0.34 × 0.10 × | 0.10 × 0.10 × |
| (mm³) | 0.02 | 0.11 | 0.09 | 0.10 |
| Symmetry | cubic | hexagonal | hexagonal | hexagonal |
| Space group | P2₁3 | P3₁ | P3₂ | R3 |
| | (no198) | | | |
| Cell | a = b = c | a = b = | a = b = | a = b = |
| dimensions | 22.1316(6) | 15.3369(5) | 15.3445(5) | 15.4681(9) |
| (Å) | α = β = γ = 90° | c = | c = 35.1273 | c = 36.345 |
| | | 35.0712(12) | (12) | (5) |
| | | α = β = 90° | α = β = 90° | α = β = 90° |
| | | γ = 120° | γ = 120° | γ = 120° |
| Volume (Å³) | 10840.2(4) | 7144.3(2) | 7162.8(3) | 7530.9(14) |
| Z | 4 | 3 | 3 | 3 |
| Formula | 2784.55 | 2144.44 | 2139.65 | 2339.75 |
| weight | | | | |
| Density | 1.706 | 1.495 | 1.488 | 1.548 |
| (Mgm⁻³) | | | | |
| Absorption | 2.983 | 1.453 | 1.449 | 1.366 |
| coefficient | | | | |
| (mm⁻¹) | | | | |
| No. of | 12879 | 147979 | 91675 | 35218 |
| reflections | | | | |
| No. of | 5319 | 28170 | 21814 | 3860 |
| unique | | | | |
| reflections | | | | |
| R(int) | 0.053 | 0.048 | 0.043 | 0.082 |
| 2θ (°) | 49.42 | 60.36 | 54.96 | 51.34 |
| R₁ for data | 0.0681 | 0.0454 | 0.0618 | 0.0679 |
| with I > 2σ(I) | | | | |
| R₁ for all | 0.1219 | 0.0528 | 0.0645 | 0.0847 |
| data | | | | |
| Goodness of | 1.027 | 1.060 | 1.178 | 1.088 |
| fit | | | | |
| Largest | 1.639 | 2.110 | 3.986 | 1.112 |
| electron peak | | | | |
| (e Å⁻³) | | | | |

### Example 21

### Physical Characterization of the Lanthanide Clusters of this Invention

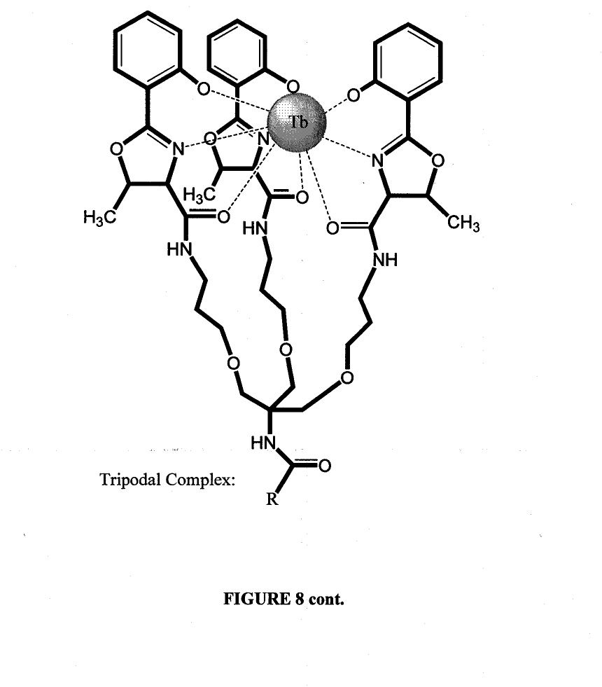

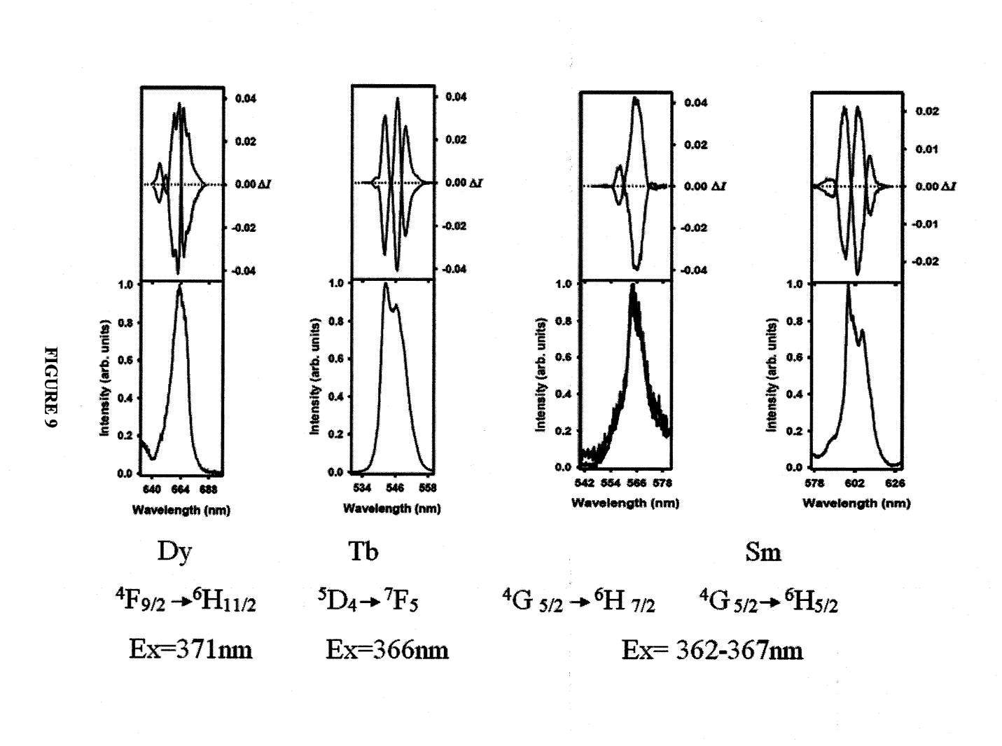

Amplified fluorescence vs. tripodes (FIG. 8).

The luminescene intensity of a solution of 0.075 mM of mono terbium tripodal (D-cis) complex was compared to that of a solution of 0.025 mM of D-cis 3Tb POxA cluster irradiated at 355 nm. Emission was measured in the range 450-650 nm. Maximum absorption of both compounds was observed at 548 nm. The intensity of the cluster spectrum was higher by two orders of magnitude.

CD Measurements.

Quartz Cuvette of 1 cm filled with spectroscopic MeOH was measured as a baseline. D-cis (4R,5R) and L-cis (4S,5S) POxA ligands 0.1 mM minus baseline were measured. CD spectra of all other enantiomer pairs (ligands and clusters) were performed in a similar manner. FIGS. 4C, 6 and 9 demonstrate the opposite chirality of two corresponding enntiomers (L and D).

Water Solubility.

Water solubility-Both 3Tb and 3GD clusters substituted by sulfonate where prepared. Both where water soluble with the 3Tb exhibiting intense characteristic green luminescence in water for several weeks. (confined structures).

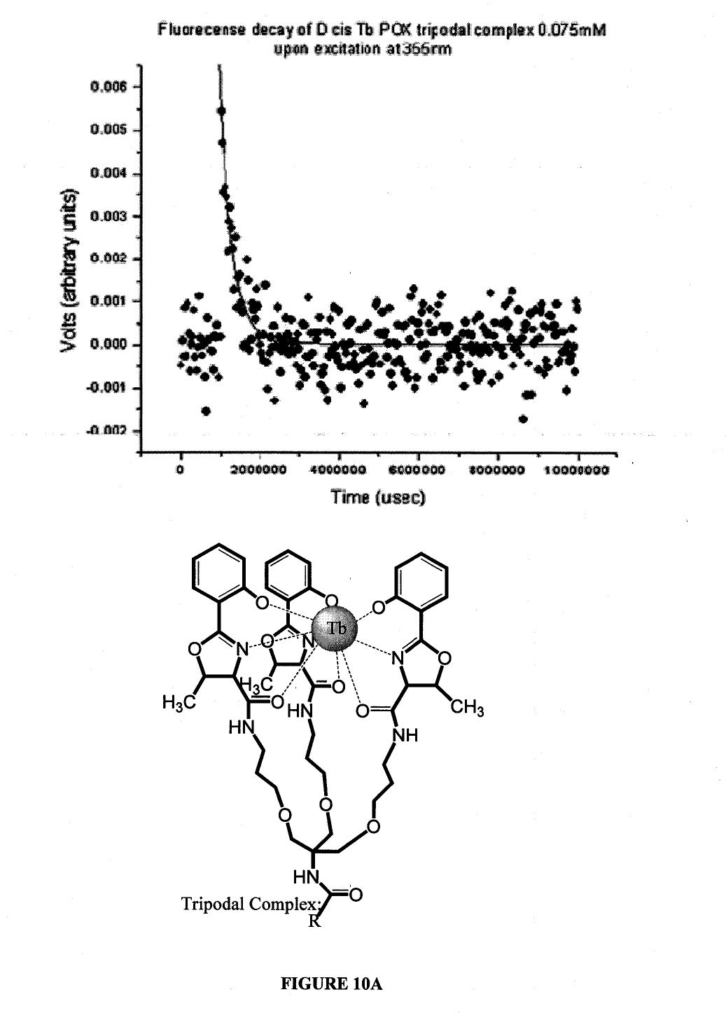

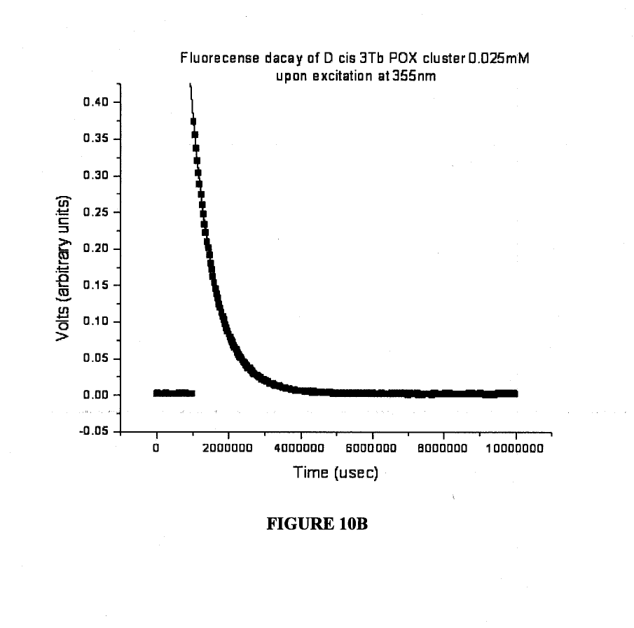

Thermodynamic Stability (FIG. 10).

Thermodynamic stability was determined by measuring the excited state half-life decay of two systems: an emissive mono lanthanide tripodal complex as a reference, and an emissive 3Tb cluster. The half-life time measurements were examined in 10% exposure, 1 μsec delay and 50 μsec band width. D-cis (4R,5R) 3Tb POxA cluster 0.025 mM was measured and compared to the life-time of D-cis(4R,5R) Tb tripodal complex at two concentrations 0.025 mM and 0.075 mM. The life-time of the cluster doubles that of the tripodal reference compound.

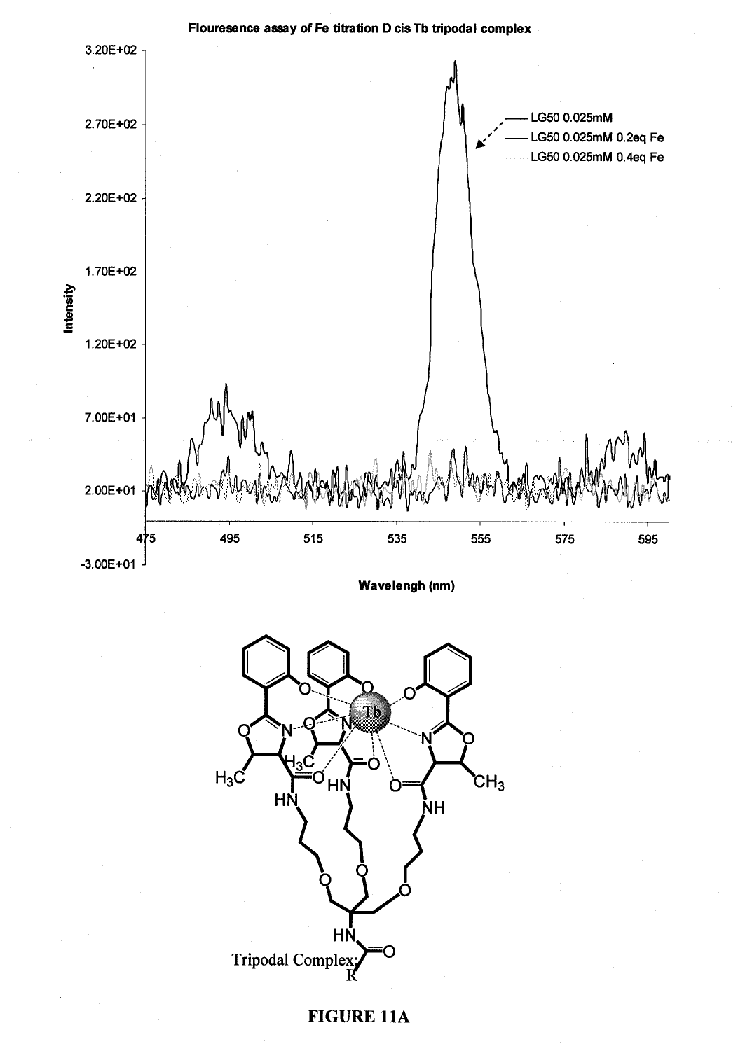

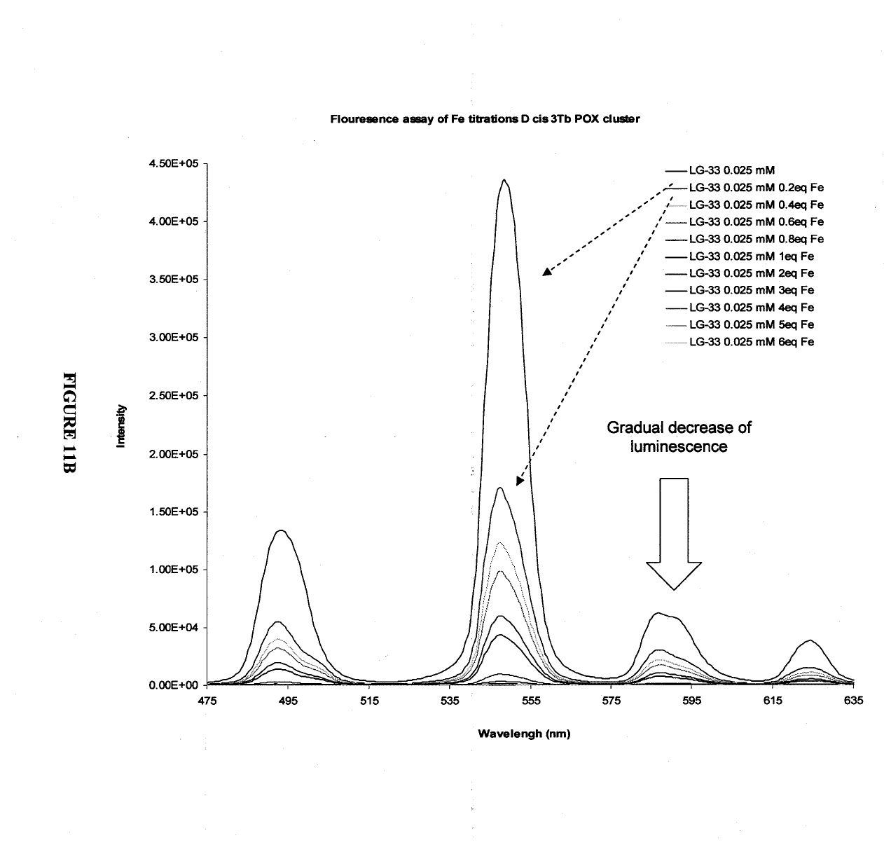

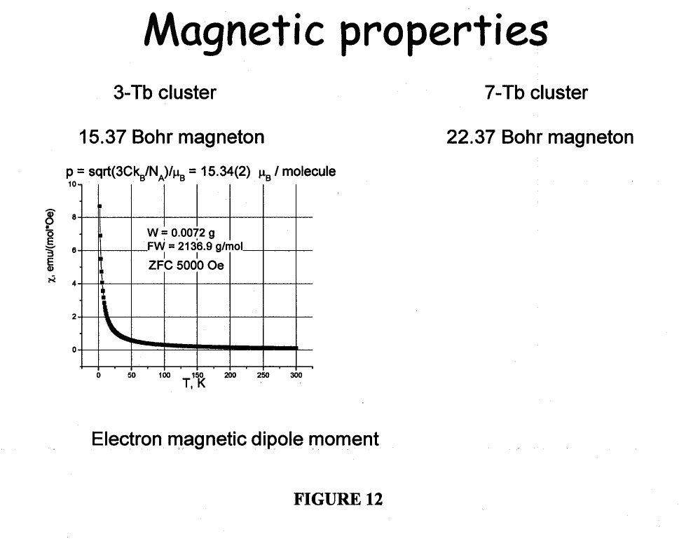

Kinetic Stability (FIG. 11).

Iron titrations of D-cis 3Tb POxA cluster 0.025 mM and D-cis Tb tripodal complex 0.025 mM were measured.

The iron (III) caused a metal exchange, and consequently quenched the lanthanide luminescene. The amount of iron(III) needed for full quenching is a measure for kinetic stability.

After 0.4 eq iron (III) the lanthanide complex was quenched, while for the cluster more than 3eq iron (III) were needed in order to quench it. Thus, the lanthanide cluster is kinetically stable as compared to the complex.

Half-life-time measurements were examined in 10% exposure, 1 μsec delay and 50 μsec band width.

Fe(III) solution was prepared from 1000 ppm Fe(III) solution in HCl diluted with MeOH.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A multinuclear lanthanide chiral cluster comprising phenyl-oxazoline-amide (POxA) ligand or salt thereof represented by the structure of formula IA: 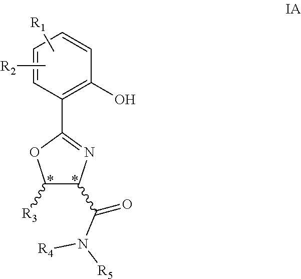
and lanthanide(III) ions;
wherein,
R₁ is hydrogen, alkyl, alkenyl, alkyldiazo, aryldiazo, alkynyl, aryl, halogen, —C≡C-Ph-R, —N═N-Ph-R, CN, NH₂, OH, N₃, NO₂, COON, COOR, SO₃H, SO₃R, SO₂NHR, O-alkyl, alkylamino, haloalkyl, or R₁ and R₂ combine together to form a 5-7 membered ring; wherein said 5-7 membered ring is saturated or unsaturated cycloalkyl or heterocycle; wherein said alkyl, alkenyl, alkyldiazo, aryldiazo, alkynyl, aryl and saturated or unsaturated cycloalkyl or heterocycle is substituted or unsubstituted;
R₂ is hydrogen, alkyl, alkenyl, alkyldiazo, aryldiazo, alkynyl, aryl, halogen, CN, NH₂, OH, N₃, NO₂, COOH, COOR, SO₃H, SO₃R, SO₂NHR, O-alkyl, alkylamino, haloalkyl, or R₁ and R₂ combine together to form a 5-7 membered ring; wherein said 5-7 membered ring is saturated or unsatureated cycloalkyl or heterocycle; wherein said alkyl, alkenyl, alkyldiazo, aryldiazo, alkynyl aryl, and saturated or unsaturated cycloalkyl or heterocycle is substituted or unsubstituted;
R₃ is alkyl, aryl, alkenyl, alkyldiazo, aryldiazo, alkynyl, halogen, CN, NH₂, OH, N₃, NO², COOH, SO₃H, SO₃R, SO₂NHR, COOR, O-alkyl, alkylamino or haloalkyl; wherein said alkyl, alkenyl, alkyldiazo, aryldiazo, alkynyl and aryl is substituted or unsubstituted;
R₄ is alkyl, alkenyl, alkyldiazo, aryldiazo, alkynyl, polyethylene glycol (PEG), sugars, glucose, manse, proteins, antibody, peptide, —CHR′COR, saturated or unsaturated cycloalkyl or heterocycle, or R₄ and R₅ combine together with the nitrogen to form a 5-7 membered ring; wherein said 5-7 membered ring is saturated or unsaturated cycloalkyl or heterocycle; wherein said alkyl, alkenyl, alkyldiazo, aryldiazo, alkynyl and saturated or unsaturated cycloalkyl or heterocycle is substituted or unsubstituted;
R₅ is hydrogen, alkyl, alkenyl or alkynyl, or R₃ and R₄ combine together with the nitrogen to form a 5-7 membered ring; wherein said 5-7 membered ring is saturated or unsaturated cycloalkyl or heterocycle;
R is hydrogen, alkyl, alkylamine, OH—N(Alkyl)₂, alkenyl, alkynyl or saturated or unsaturated cycloalkyl or heterocycle; wherein said alkyl, alkenyl, alkynyl and saturated or unsaturated cycloalkyl or heterocycle is substituted or unsubstituted; and
R′ is an amino acid side chain.

2. The chiral structure of claim 1, wherein R₁ is H, halogen, —C≡C, SO₃H, SO₃Na, NH₂ or NO₂.

3. The chiral cluster of claim 1, wherein R₃ is an alkyl.

4. The chiral cluster of claim 1. wherein R₄ is an alkyl or saturated or unsaturated cycloalkyl or heterocycle.

5. (canceled)

6. The chiral structure of claim 1, wherein R₅ is hydrogen.

7. The chiral cluster of claim 1, wherein a three lanthanide cluster comprises three lanthanides which coordinate to said POxA ligand as presented by the structure of formula Ila and the structure of formula IIb in equal ratios: 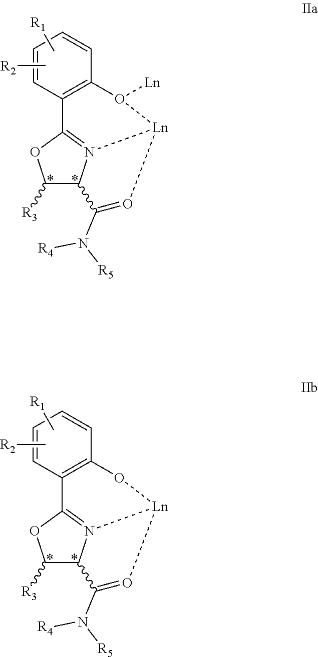
wherein,
Ln is a lanthanide(III) ion;
oxygen bridges coordinate between said lanthanide ions; and said cluster further comprises one or more oxygen based ligands, one or more halogens, or combination thereof.

8. The chiral cluster of claim 1, wherein a seven lanthanide cluster comprises seven lanthanides which coordinate to said POxA ligand as presented by the structure of formula IIa, IIb and IIc in equal ratios: 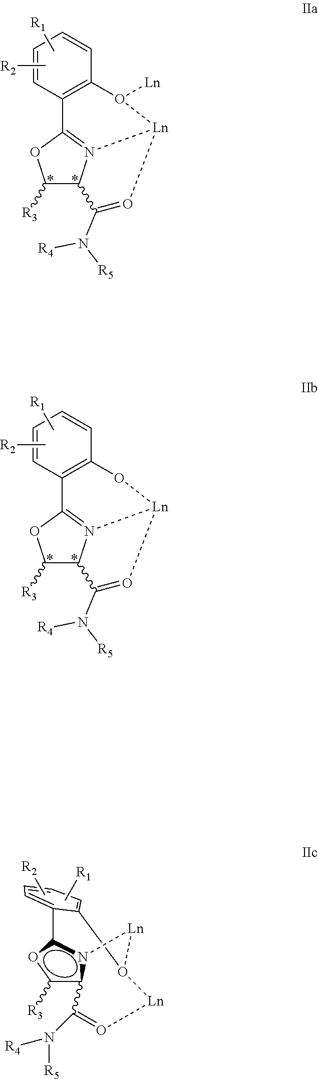
wherein,
Ln is a lanthanide(III) ion:
oxygen bridges coordinate between said lanthanide ions: and said cluster further comprises one or more oxygen based ligands, one or more halogens, or combination thereof.

9. The chiral cluster of claim 7, wherein said oxygen based ligand is alcohol or water.

10. The chiral cluster of claim 1, wherein said lanthanide is La(III), Pr(III), Pm(III), Sm(III), Eu(III), Gd(III), Tb(III), Dy(III), Ho(III), Er(III), Yb(III) or Lu(III).

11. The chiral cluster of claim 1, wherein said lanthanide is the same or different.

12. The chiral cluster of claim 1, wherein said phenyl-oxazoline-amide (POxA) ligand is a cis isomer with 4R,5R or 4S,5S chiral centers and said cluster is a three lanthanides cluster with six phenyl-oxazoline-amide (POxA) ligands.

13. The chiral cluster of claim 1, wherein said phenyl-oxazoline-amide (POxA) ligand is a trans isomer with 4R,5S or 4S, 5R chiral centers and said cluster is a seven lanthanides cluster with nine phenyl-oxazoline-amide (POxA) ligands.

14. (canceled)

15. (canceled)

16. The chiral cluster of claim 1, wherein said cluster further comprises oxygen bridges between the lanthanides.

17. The chiral cluster of claim 1, wherein said cluster is emitting circularly polarized luminescence (CPL).

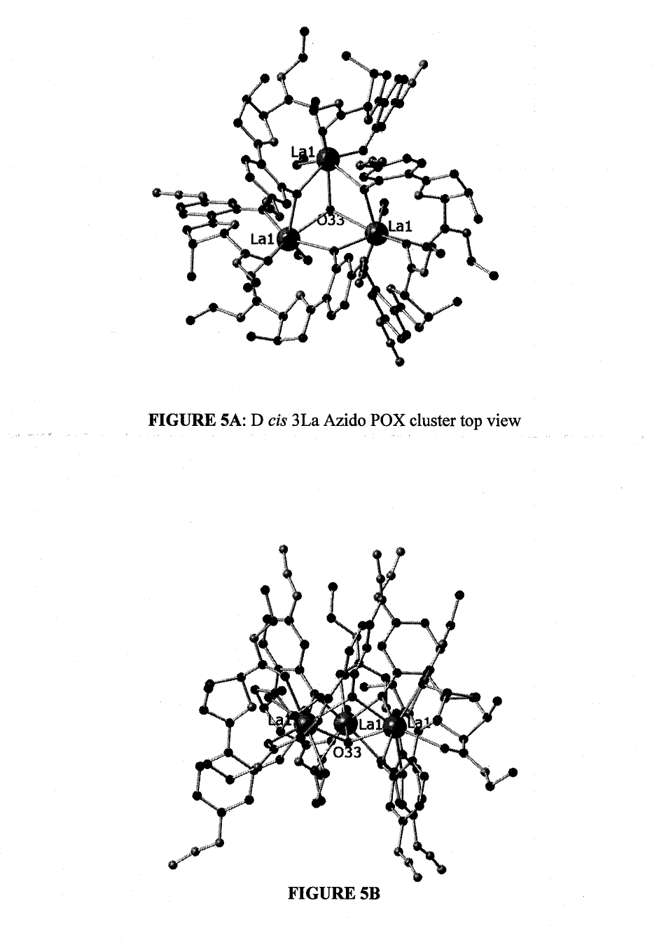

18. A crystalline structure of said chiral cluster of claim 7, wherein said three lanthanide cluster is represented by the structures of FIGS. 2B, 2C, 3A, 3B, 4A, 4B, 5A or 5B.

19. A crystalline structure of said chiral cluster of claim 8, wherein said seven lanthanide cluster is represented by the structures of FIG. 2A.

20. A multinuclear lanthanide chiral cluster comprising phenyl-oxazoline-amide (POxA) ligand or salt thereof represented by the structure of formula IIIA: 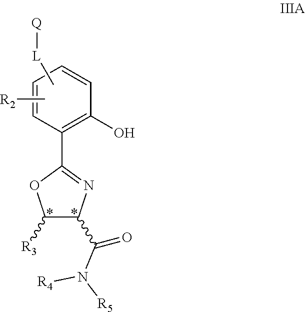
and lanthanide (III) ions;
wherein,
Q is a sensor, monomeric building-block for polymerization, a polymer, chromophore, surface adhesive group or combination thereof;
L is a bond or a linker;
R₂ is hydrogen, alkyl, alkenyl, alkyldiazo, aryldiazo, alkynyl, aryl, halogen, —C≡C-Ph-R, —N═N-Ph-R, CN, NH₂, OH, N₃, NO₂, COOH, COOR, SO₃H, SO₃R, SO₂NHR, O-alkyl, alkylamino, haloalkyl, or R₁ and R₂ combine together to form a 5-7 membered ring; wherein said 5-7 membered ring is saturated or unsatureated cycloalkyl or heterocycle; wherein said alkyl, alkenyl, alkyldiazo, aryldiazo, alkynyl, aryl and saturated or unsaturated cycloalkyl or heterocycle is substituted or unsubstituted;
R₃ is alkyl, aryl, alkenyl, alkyldiazo, aryldiazo, alkynyl, halogen, CN, NH₂, OH, N₃, NO₂, COOH, SO₃H, SO₃R, SO₂NHR, COOR, O-alkyl, alkylamino or haloalkyl; wherein said alkyl, alkenyl, alkyldiazo, aryldiazo, alkynyl and aryl is substituted or unsubstituted;
R4 is alkyl, alkenyl, alkyldiazo, aryldiazo, alkynyl, polyethylene glycol (PEG), sugars, glucose, manse, galactose, proteins, antibody, peptide, -CHR′COR, saturated or unsaturated cycloalkyl or heterocycle; or R₄ and R₅ combine together with the nitrogen to form a 5-7 membered ring; wherein said 5-7 membered ring is saturated or unsaturated cycloalkyl or heterocycle; wherein said alkyl, alkenyl, alkyldiazo, aryldiazo, alkynyl and saturated or unsaturated cycloalkyl or heterocycle is substituted or unsubstituted;
R₅ is hydrogen, alkyl, alkenyl or alkynyl or R₅ and R₄ combine together with the nitrogen to form a 5-7 membered ring; wherein said 5-7 membered ring is saturated or unsaturated cycloalkyl;
R is hydrogen, alkyl, alkylamine, OH, —N(Alkyl)₂, alkenyl, alkynyl or saturated or unsaturated cycloalkyl or heterocycle; wherein said alkyl, alkenyl, alkynyl and saturated or cycloalkyl or heterocycle is substituted or unsubstituted; and
R′ is an amino acid side chain.

21. The chiral structure of claim 20, wherein R₅ is hydrogen.

22. The chiral cluster of claim 20, wherein a three lanthanide cluster comprises three lanthanides which coordinate to said POxA ligand as presented by the structure of formula IVa and the structure of formula IVb in equal ratios: 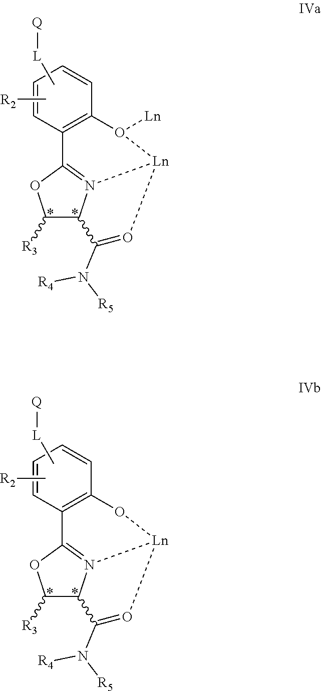
wherein,
Ln is a lanthanide(III) ion;
oxygen bridges coordinate between said lanthanide ions; and said cluster further comprises one or more oxygen based ligands, one or more halogens, or combination thereof.

23. The chiral cluster of claim 20, wherein a seven lanthanide cluster comprises seven lanthanides which coordinate to said POxA ligand as presented by the structure of formula IVa, IVb and IVc in equal ratios: 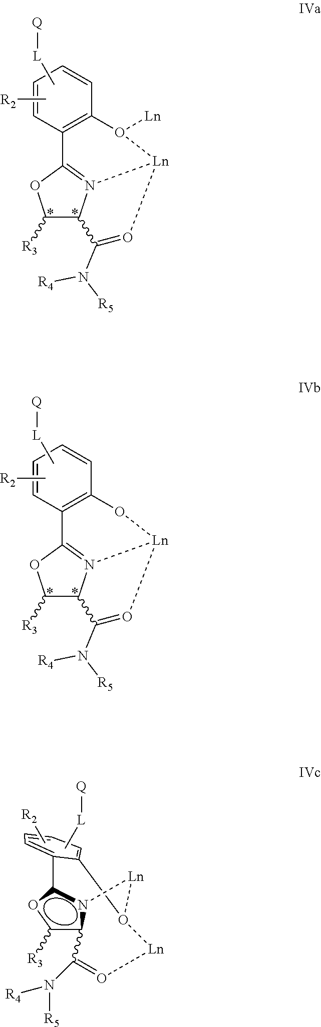
wherein,
Ln is a lanthanide(III) ion;
oxygen bridges coordinate between said lanthanide ions: and said cluster further comprises one or more oxygen based ligands, one or more halogens, or combination thereof.

24. The chiral cluster of claim 22, wherein said oxygen based ligand is alcohol or water.

25. The chiral cluster of claim 20, wherein said lanthanide is La(III), Ce(III), Pr(III), Nd(III), Pm(III), Sm(III), Eu(III), Tb(III), Dy(III), Ho(III), Er(III), Tm(III), Yb(III) or Lu(III), wherein said lanthanide is the same or different.

26. (canceled)

27. The chiral cluster of claim 20, wherein said phenyl-oxazoline-amide (POxA) ligand is a cis isomer with 4R,5R or 4S,5S chiral centers and said cluster is a three lanthanides cluster with six phenyl-oxazoline-amide (POxA) ligands.

28. The chiral cluster of claim 20, wherein said phenyl-oxazoline-amide (POxA) ligand is a trans isomer with 4R,5S or 4S,5R chiral centers and said cluster is a seven lanthanides cluster with nine phenyl-oxazoline-amide (POxA) ligands.

29. (canceled)

30. (canceled)

31. A biomarker comprising said multinuclear lanthanide chiral cluster of claim 20.

32. A method of identifying and quantifying a biomolecule in a sample, comprising:
(i) contacting a sample comprising a biomolecule with a chiral cluster of claim 20; wherein said biomolecule is selected from peptides, proteins, oligonucleotides, nucleic acids, oligosaccharides, polysaccharides, glycoproteins, phospholipids and enzymes; and
(ii) measuring luminescence following interaction between said biomolecule and said chiral cluster;
thereby identifying and quantifying said biomolecule in said sample.

33. The method of claim 32, wherein said measuring is directly of said lanthanide(III).

34. The method of claim 32, wherein Q of said cluster comprises hyaluronic acid, and thereby identifying and quantifying a CD44 receptor.

35. The method of claim 32, wherein Q of said cluster comprises Arg-Gly-Asp (RGD), and thereby identifying and quantifying a integrin receptors.

36. The method of claim 32, wherein Q of said cluster comprises glucosamine, and thereby identifying and quantifying glucose.

37. A method of identifying and quantifying a metal ion in a sample, comprising:
(i) contacting a sample comprising a metal ion with a chiral cluster of claim 20; and
(ii) measuring luminescence following interaction between said metal ion and said chiral cluster;
thereby identifying and quantifying said metal ion in said sample.

38. The method of claim 37, wherein Q of said cluster comprises hydroxamate, and thereby identifying and quantifying iron(III).

39. The method of claim 37, wherein Q of said cluster comprises bipyridyl, and thereby identifying and quantifying Ru(II) or Cr(III).

40. The method of claim 37, wherein Q of said cluster comprises 8-hydroxyquinoline and thereby identifying and quantifying Al(III).

41. A contrast agent in Magnetic Resonance Imaging (MRI) comprising said multinuclear lanthanide chiral cluster of claim 1.

42. An inkjet printing comprising said multinuclear lanthanide chiral cluster of claim 1.

43. An optical fiber comprising said multinuclear lanthanide chiral cluster of claim 1.

44. A liquid crystal display comprising said multinuclear lanthanide chiral cluster of claim 1.

45. A method of coding and reading said coded information comprising waiting a code with said chiral cluster of claim 1, and reading said code by measuring its magnetic properties, its luminescence in visible or NIR or by measuring its emission light for circular polarized luminescence (CPL).

46. The chiral cluster of claim 8, wherein said oxygen based ligand is alcohol or water.

47. The chiral cluster of claim 23, wherein said oxygen based ligand is alcohol or water.

48. A contrast agent in Magnetic Resonance Imaging (MRI) comprising said multinuclear lanthanide chiral cluster of claim 20.

49. An inkjet printing comprising said multinuclear lanthanide chiral cluster of claim 20.

50. An optical fiber comprising said multinuclear lanthanide chiral cluster of claim 20.

51. A liquid crystal display comprising said multinuclear lanthanide chiral cluster of claim 20.

52. A method of coding and reading said coded information comprising writing a code with said chiral cluster of claim 20, and reading said code by measuring its magnetic properties, its luminescence in visible or NIR or by measuring its emission light for circular polarized luminescence (CPL).
